(19) United States
(12) Patent Application Publication
(10) Pub. No.: US 2019/0292494 A1
(43) Pub. Date: Sep. 26, 2019

(54) STABILIZATION OF ENZYMES IN COMPOSITIONS

(71) Applicant: BASF SE, Ludwigshafen am Rhein (DE)

(72) Inventors: Stefan Jenewein, Ludwigshafen (DE); Max-Philipp Fischer, Stuttgart (DE); Hans Wolfgang Hoeffken, Ludwigshafen (DE); Janosch Harald Achenbach, Ludwigshafen (DE); Oliver Spangenberg, Ludwigshafen (DE); Allan Francis Cunningham, Magden (CH); Jesper Nielsen, San Diego, CA (US)

(21) Appl. No.: 16/465,677

(22) PCT Filed: Nov. 21, 2017
(86) PCT No.: PCT/EP2017/079878

§ 371 Date: May 31, 2019

(30) Foreign Application Priority Data
Dec. 1, 2016 (EP) 16201739.6

(51) Int. Cl. C11D 3/386; C11D 3/20; C11D 3/16; C12N 9/96; C11D 17/00

(52) U.S. Cl. CPC C11D 3/38663; C11D 3/38618; C11D 3/2044; C12Y 304/21062; C12N 9/96; C11D 17/0039; C11D 3/166

(57) ABSTRACT

A composition comprising component (a) at least one phenyl boronic acid and component (b) pentane-1,2-diol and optionally one or more further diols wherein the composition is liquid at 20° C. and 101.3 kPa. Said composition stabilizes serine protease.

This invention relates to compositions comprising at least one boron-containing compound and pentane-1,2-diol. Said composition may optionally comprise one or more further diols. The invention also relates to detergent compositions comprising said composition, at least one enzyme selected from serine proteases and at least one detergent component.

Enzymes are generally produced commercially as a liquid concentrate, frequently derived from a fermentation broth. The enzyme tends to be destabilized if it remains in an aqueous environment and so it is conventional practice to convert it to an anhydrous form: aqueous concentrates may be lyophilized or spray-dried e.g. in the presence of a carrier material to form aggregates. However, such particles often need to be “dissolved” prior to use, especially when enzymes are destined to be part of liquid formulations.

A significant field of application for enzymes are detergent compositions. Detergent compositions comprising enzymes have to fulfill some minimal requirements: 1) exhibit a certain shelf life and 2) have excellent cleaning properties for various soiling, including enzyme-sensitive stains. The latter aspect is directly influenced by the shelf life of enzymes, as the goal is to maintain the excellent cleaning properties of enzymes in detergent compositions over an extended period of time, e.g. during storage such detergent compositions.

Enzymes are incorporated in detergent compositions either as solid or liquid compositions. Whenever enzyme compositions are liquid, enzymes need to be stabilized to maintain their activity. A protease inhibitor may be used for this purpose, since proteolytic digestion is a major cause for activity loss.

Boric acid and boronic acids are known to reversibly inhibit proteolytic enzymes. A discussion of the inhibition of one serine protease, subtilisin, by boronic acid is provided in Molecular & Cellular Biochemistry 51, 1983, pp. 5-32. For reactivation, this inhibitor needs to be removed prior or during application, which can be done for example by dilution.

WO 92/19709 discloses protease-containing liquid detergent compositions and discusses the issue of degradation of additional enzymes in the composition by the proteolytic enzyme upon storage of the product. The disclosure of WO 92/19709 is directed to the problem of liquid detergent compositions built with alpha-hydroxyacid, as boric acids and its derivatives, which were already known to reversibly inhibit proteolytic enzymes, appear to complex with the builder and consequently do not sufficiently inhibit the proteolytic enzyme. The liquid detergents disclosed therein comprise: (a) a mixture of boric acids or its derivatives and vicinal polyols, (b) proteolytic enzyme, (c) detergent-compatible second enzyme, (d) anionic and/or nonionic surfactant, and (e) alpha-hydroxyacid builder. It is disclosed that boric acid or polyol by themselves do not provide sufficient stability to lipase in a heavy-duty liquid composition containing a proteolytic enzyme. The lipase stability is disclosed to be improved in the presence of protease by using a mixture of boric acid and (1) propane-1,2-diol, (2) butane-1,2-diol, (3) hexane-1,2-diol, (4) sorbitol, (5) sucrose and (6) mannose for stabilization of protease.

EP 0381262 discloses mixtures of proteolytic and lipolytic enzymes in a liquid environment. The stability of the lipolytic enzyme is said to be improved by the addition of a stabilizer system comprising a boron compound and a polyol. The polyol contains only C, H and O atoms and should have at least two vicinal hydroxyl groups. Typical examples of suitable polyols are said to be D-mannitol, sorbitol and 1,2-benzenediol.

The present invention is based on the problem of providing a composition which is effective in reversible inhibition of enzymatic activity, preferably reversible inhibition of proteolytic activity. Furthermore, said composition shall be effective when comprised in liquid compositions comprising at least one serine protease.

The problem was solved by providing a composition comprising

component (a): at least one boron-containing compound, and

component (b): pentane-1,2-diol and optionally one or more further diols,

wherein the composition is liquid at 20° C. and 101.3 kPa.

In one embodiment, one or more further diols optionally comprised in component (b) is selected from water-miscible diols other than pentane-1,2-diol.

In one embodiment, component (a) is selected from boronic acid or its derivatives, preferably BBA and 4-FPBA.

In one embodiment, at least one boron-containing compound comprised in component (a) is selected from phenyl-boronic acid or its derivatives, such as BBA and 4-FPBA.

In one embodiment, the composition comprises an additional component (c) which comprises at least one serine protease and optionally one or more further enzymes.

In one embodiment, one or more further enzymes comprised in component (c) is selected from proteolytic enzymes other than serine proteases and/or lipases and/or amylases and/or cellulases.

In one embodiment, the composition has a pH in the range of 7 to 11.5.

In one embodiment, the present invention provides a (method of) use of pentane-1,2-diol [component (b)] in the presence of at least one boron-containing compound [component (a)] in compositions comprising at least one enzyme, wherein at least one enzyme is selected from serine proteases [component (c)] for stabilization of serine protease(s).

In one embodiment, the present invention provides a (method of) use of pentane-1,2-diol [component (b)] in the presence of at least one boron-containing compound [component (a)] in compositions comprising at least one enzyme, wherein at least one enzyme is selected from serine proteases [component (c)] for improvement of stabilization of serine protease(s).

In one embodiment, the invention provides a microcapsule comprising

component (a): at least one boron-containing compound, and

component (b): pentane-1,2-diol and optionally one or more further diols, and

component (c): at least one serine protease and optionally one or more further enzymes,

wherein components (a), (b) and (c) are part of the core composition of the microcapsule.

In one embodiment, the core composition is liquid at 20° C. and 101.3 kPa.

The invention provides a method of preparing a composition comprising mixing in no specified order in one or more steps

component (a): at least one boron-containing compound, and

component (b): pentane-1,2-diol and optionally one or more further diols, and

optionally component (c): at least one serine protease and optionally one or more further enzymes, and

optionally component (d): one or more detergent components.

In one embodiment, the invention provides a method of preparing a detergent composition comprising components (a), (b), (c) and (d).

The invention further relates to a detergent composition comprising

component (a): at least one boron-containing compound, and

component (b): pentane-1,2-diol and optionally one or more further diols, and

component (c): at least one serine protease and optionally one or more further enzymes, and component (d): one or more detergent components

The detergent composition may be solid or liquid.

In one embodiment, the detergent composition comprises component (a) in effective amounts, component (b) in amounts in the range of 2% to 50% w/w relative to the total weight of the composition, and component (c) in amounts in the range of 0.01 g/L to 20 g/L.

The invention provides a method for removing stains comprising contacting an enzyme-sensitive stain with a composition comprising

component (a): at least one boron-containing compound, and

component (b): pentane-1,2-diol and optionally one or more further diols, and

component (c): at least one serine protease and optionally one or more further enzymes, and optionally component (d): one or more detergent components.

In one embodiment, the method for removing stains comprises contacting an enzyme sensitive stain with a detergent composition comprising

component (a): at least one boron-containing compound and

component (b): pentane-1,2-diol and optionally one or more further diols, and

component (c): at least one serine protease and optionally one or more further enzymes, and component (d): one or more detergent components.

The invention provides a method for cleaning comprising contacting soiled material with a detergent composition comprising

component (a): at least one boron-containing compound and

component (b): pentane-1,2-diol and optionally one or more further diols, and

component (c): at least one serine protease and optionally one or more further enzymes, and component (d): one or more detergent components.

The method of cleaning may be laundering or hard surface cleaning.

In one embodiment, the soiled material comprises at least one enzyme-sensitive stain.

### DETAILED DESCRIPTION

Enzymes herein are mainly identified by polypeptide sequences.

Abbreviations for single amino acids used within this invention are as follows:

| | | |
|---|---|---|
| Alanine | Ala | A |
| Arginine | Arg | R |
| Asparagine | Asn | N |
| Aspartic acid | Asp | D |
| Cysteine | Cys | C |
| Glutamic acid | Glu | E |
| Glutamine | Gln | Q |
| Glycine | Gly | G |
| Histidine | His | H |
| Isoleucine | Ile | I |
| Leucine | Leu | L |
| Lysine | Lys | K |
| Methionine | Met | M |
| Phenylalanine | Phe | F |
| Proline | Pro | P |
| Serine | Ser | S |
| Threonine | Thr | T |
| Tryptophan | Trp | W |
| Tyrosine | Tyr | Y |
| Valine | Val | V |

The accepted IUPAC single letter or three letter amino acid abbreviation is employed. “Parent” sequence (of a parent protein or enzyme, also called “parent enzyme”) is the starting sequences for introduction of changes (e.g. by introducing one or more amino acid substitutions) of the sequence resulting in “variants” of the parent sequences. The term parent enzyme (or parent sequence) includes
1.wild-type enzymes (sequences) and
2. variant sequences (enzymes) which are used as starting sequences for introduction of (further) changes.

The term “enzyme variant” or “sequence variant” or “variant enzyme” differ from parent enzymes in their amino acid sequence to a certain extent; however, variants normally are requested at least to maintain the enzyme properties of the respective parent enzyme. Variant enzymes may have at least the same enzymatic activity when compared to the respective parent enzyme or variant enzymes may have increased enzymatic activity when compared to the respective parent enzyme.

In describing the variants of the present invention, the nomenclature described as follows is used:

Substitutions are described by providing the original amino acid followed by the number of the position within the amino acid sequence, followed by the substituted amino acid. For example, the substitution of histidine at position 120 with alanine is designated as “His120Ala” or “H120A”.

Deletions are described by providing the original amino acid followed by the number of the position within the amino acid sequence, followed by *. Accordingly, the deletion of glycine at position 150 is designated as “Gly150*” or G150*”. Alternatively, deletions are indicated by e.g. “deletion of D183 and G184”.

Insertions are described by providing the original amino acid followed by the number of the position within the amino acid sequence, followed by the original amino acid and the additional amino acid. For example, an insertion at position 180 of lysine next to glycine is designated as “Gly180GlyLys” or “G180GK”. When more than one amino acid residue is inserted, such as e.g. a Lys and Ala after Gly180 this may be indicated as: Gly180GlyLysAla or G195GKA.

In cases where a substitution and an insertion occur at the same position, this may be indicated as S99SD+S99A or in short S99AD.

In cases where an amino acid residue identical to the existing amino acid residue is inserted, it is clear that degeneracy in the nomenclature arises. If for example a glycine is inserted after the glycine in the above example this would be indicated by G180GG.

Variants comprising multiple alterations are separated by “+”, e.g. “Arg170Tyr+Gly195Glu” or “R170Y+G195E” representing a substitution of arginine and glycine at positions 170 and 195 with tyrosine and glutamic acid, respectively. Alternatively, multiple alterations may be separated by space or a comma e.g. R170Y G195E or R170Y, G195E respectively.

Where different alterations can be introduced at a position, the different alterations are separated by a comma, e.g. “Arg170Tyr, Glu” represents a substitution of arginine at position 170 with tyrosine or glutamic acid. Alternatively different alterations or optional substitutions may be indicated in brackets e.g. Arg170[Tyr, Gly] or Arg170{Tyr, Gly} or in short R170 [Y,G] or R170 {Y, G}.

Variants of the parent enzyme molecules may have an amino acid sequence which is at least n % identical to the amino acid sequence of the respective parent enzyme having enzymatic activity with n being an integer between 10 and 100. In one embodiment, variant enzymes are at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% identical when compared to the full length polypeptide sequence of the parent enzyme. In one embodiment, variant enzymes which are n % identical when compared to a parent enzyme, have enzymatic activity. “Identity” in relation to comparison of two amino acid sequences herein is calculated by dividing the number of identical residues by the length of the alignment region which is showing the shorter sequence over its complete length. This value is multiplied by 100 gives “%-identity”.

To determine the %-identity between two amino acid sequences (i.e. pairwise sequence alignment), two sequences have to be aligned over their complete length (i.e. global alignment) in a first step. For producing a global alignment of two sequences, any suitable computer program, like program “NEEDLE” (The European Molecular Biology Open Software Suite (EMBOSS)), program “MATGAT” (Campanella, J. J, Bitincka, L. and Smalley, J. (2003), BMC Bioinformatics, 4:29), program “CLUSTAL” (Higgins, D. G. and Sharp, P. M. (1988), Gene, 73, 237-244) or similar programs may be used. In lack of any program, sequences may also be aligned manually.

After aligning two sequences, in a second step, an identity value shall be determined from the alignment. Depending on the applied method for %-identity calculation, different %-identity values can be calculated from a given alignment. Consequently, computer programs which create a sequence alignment, and in addition calculate %-identity values from the alignment, may also report different %-identity values from a given alignment, depending which calculation method is used by the program.

Therefore, the following calculation of %-identity according to the invention applies:

%-identity=(identical residues/length of the alignment region which is showing the shorter sequence over its complete length)*100.

The calculation of %-identity according to the invention is exemplified as follows (the sole purpose of Seq 1 and Seq 2 is to demonstrate calculation according to the invention; besides this purpose, said sequences are not inventive or functionally meaningful):

Seq 1:
TTTTTTAAAAAAAACCCCHHHCCCCAAARVHHHHHTTTTTTTT-
length: 43 amino acids
Seq 2:
TTAAAAAAAACCCCHHCCCCAAADLSSHHHHHTTTT-
length: 36 amino acids

Hence, the shorter sequence is sequence 2.

Producing a pairwise global alignment which is showing both sequences over their complete lengths results in

| | |
|---|---|
| Seq 1: | TTTTTTAAAAAAAACCCCHHHCCCCAAARV--HHHHHTTTTTTTT |
| | \|\|\|\|\|\|\|\|\|\|\|\|\|\| \|\|\|\|\|\|\|\|\| : \|\|\|\|\|\|\|\|\| |
| Seq 2: | ----TTAAAAAAAACCCC-HHCCCCAAADLSSHHHHHTTTT---- |

Producing a pairwise alignment which is showing the shorter sequence over its complete length according the invention consequently results in:

| | |
|---|---|
| Seq 1: | TTAAAAAAAACCCCHHHCCCCAAARV--HHHHHTTTT |
| | \|\|\|\|\|\|\|\|\|\|\|\|\|\| \|\|\|\|\|\|\|\|\| : \|\|\|\|\|\|\|\|\| |
| Seq 2: | TTAAAAAAAACCCC-HHCCCCAAADLSSHHHHHTTTT |

The number of identical residues is 32, the alignment length showing the shorter sequence over its complete length is 37 (one gap is present which is factored in the alignment length of the shorter sequence)

Therefore, %-identity according to the invention is: (32/37)*100=86%

A special aspect concerning amino acid substitutions are conservative mutations which often appear to have a minimal effect on protein folding resulting in substantially maintained enzyme properties of the respective enzyme variant compared to the enzyme properties of the parent enzyme. Conservative mutations are those where one amino acid is exchanged with a similar amino acid. Such an exchange most probably does not change enzyme properties. For determination of %-similarity the following applies:

Amino acid A is similar to amino acids S

Amino acid D is similar to amino acids E; N

Amino acid E is similar to amino acids D; K; Q

Amino acid F is similar to amino acids W; Y

Amino acid H is similar to amino acids N; Y

Amino acid I is similar to amino acids L; M; V

Amino acid K is similar to amino acids E; Q; R

Amino acid L is similar to amino acids I; M; V

Amino acid M is similar to amino acids I; L; V

Amino acid N is similar to amino acids D; H; S

Amino acid Q is similar to amino acids E; K; R

Amino acid R is similar to amino acids K; Q

Amino acid S is similar to amino acids A; N; T

Amino acid T is similar to amino acids S

Amino acid V is similar to amino acids I; L; M

Amino acid W is similar to amino acids F; Y

Amino acid Y is similar to amino acids F; H; W

Conservative amino acid substitutions may occur over the full length of the sequence of a polypeptide sequence of a functional protein such as an enzyme. In one embodiment, such mutations are not pertaining the functional domains of an enzyme. In one embodiment, conservative mutations are not pertaining the catalytic centers of an enzyme.

To take conservative mutations into account, a value for “similarity” of two amino acid sequences may be calculated. “Similarity” in relation to comparison of two amino acid sequences herein is calculated by dividing the number of identical residues plus the number of similar residues by the length of the alignment region which is showing the shorter sequence over its complete length. This value is multiplied by 100 gives “%-similarity”.

Therefore, the following calculation of %-similarity according to the invention applies:

%-similarity=[(identical residues+similar residues)/length of the alignment region which is showing the shorter sequence over its complete length]*100.

Using the example above with the pairwise alignment showing the shorter sequence over its complete length according the invention as follows for calculation of %-similarity:

| | |
|---|---|
| Seq 1: | TTAAAAAAAACCCCHHHCCCCAAARV--HHHHHTTTT |
| | \|\|\|\|\|\|\|\|\|\|\|\|\|\| \|\|\|\|\|\|\|\|\| : \|\|\|\|\|\|\|\|\| |
| Seq 2: | TTAAAAAAAACCCC-HHCCCCAAADLSSHHHHHTTTT |

The number of identical residues is 32, the number of similar amino acids exchanged is 1 (indicated by “:” in the alignment displayed above), the alignment length showing the shorter sequence over its complete length is 37 (one gap is present which is factored in the alignment length of the shorter sequence)

Therefore, %-similarity according to the invention is: [(32+1)/37]*100=89%

Variant enzymes comprising conservative mutations which are at least m % similar to the respective parent sequences with m being an integer between 10 and 100 are expected to have essentially unchanged enzyme properties. In one embodiment, variant enzymes are at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% similar when compared to the full length polypeptide sequence of the parent enzyme. In one embodiment, variant enzymes with m %-similarity when compared to a parent enzyme, have enzymatic activity.

Enzymes are generally produced commercially by using recombinant host cells which express the desired enzyme by cultivation of the same under conditions suitable for expression of the desired enzyme. Cultivation normally takes place in a suitable nutrient medium allowing the recombinant host cells to grow and express the desired enzyme (this process may be called fermentation herein). At the end of fermentation, fermentation broth is collected and may be further processed, wherein the fermentation broth comprises

1. A liquid fraction and

2. A solid fraction.

The desired protein or enzyme may be secreted (into the liquid fraction of the fermentation broth) or may not be secreted from the host cells (and therefore is comprised in the solid fraction of the fermentation broth). Depending on this, the desired protein or enzyme may be recovered from the liquid fraction of the fermentation broth or from cell lysates. However, the desired protein may be comprised in both, the liquid and the solid fraction of the fermentation broth.

Recovery of the desired enzyme uses methods known to those skilled in the art. Suitable methods for recovery of proteins or enzymes from fermentation broth include but are not limited to collection, centrifugation, filtration, extraction, and precipitation. The resulting enzyme fraction may be used as such in the final application if suitable or may be further purified.

For purification of enzyme a variety of methods are known in the art, including but not limited to chromatography such as ion exchange, affinity chromatography, hydrophobic chromatography, chromatofocusing, and size exclusion; electrophoretic methods such as preparative isoelectric focusing; differential solubility such as ammonium sulfate precipitation; SDS-PAGE, and extraction. Variable degrees of enzyme purity may be obtained by purification methods and any quality of the resulting enzyme product may be used in the final application if suitable. The resulting enzyme product may be liquid.

Enzymes tend to be destabilized if they remain in a liquid environment, especially if they remain in an aqueous environment. Therefore, liquid enzyme products may be stabilized by methods such as addition of chemicals (e.g. addition of boric acid to protease fractions), or liquid enzyme products may be converted to an anhydrous form by lyophilization or spray-drying e.g. in the presence of a carrier material to form aggregates.

“Enzyme properties” include, but are not limited herein to catalytic activity as such, substrate/cofactor specificity, product specificity, increased stability in the course of time, thermal stability, pH stability, chemical stability, and improved stability under storage conditions. The term “substrate specificity” reflects the range of substrates that can be catalytically converted by an enzyme.

“Enzymatic activity” means the catalytic effect exerted by an enzyme, expressed as units per milligram of enzyme (specific activity) or molecules of substrate transformed per minute per molecule of enzyme (molecular activity). Enzymatic activity can be specified by the enzymes actual function, e.g. proteases exerting proteolytic activity by catalyzing hydrolytic cleavage of peptide bonds, lipases exerting lipolytic activity by hydrolytic cleavage of ester bonds, etc.

“Increased enzymatic activity” or “improved enzymatic activity” according to the current invention relates to the increased catalytic effect exerted by a variant enzyme, when compared to the parent enzyme. Further, “increased enzymatic activity” may also relate to an improved catalytic effect resulting from a (synergistic) effect of a defined enzyme and a chemical and/or detergent component, when compared to the defined enzyme without the chemical and/or detergent component.

“Enzyme assays” are methods for measuring enzymatic activity. Enzyme assays allow to measure either the consumption of substrate or production of product over time. According to their sampling method, continuous assays (continuous measurement of enzymatic activity) can be distinguished from discontinuous assays (at a certain point in time, enzymatic activity is measured after stopping the reaction). The one skilled in the art is aware of choosing appropriate enzyme assay for a given problem.

Enzymatic activity might change during storage or operational use of the enzyme. The term “enzyme stability” according to the current invention relates to the retention of enzymatic activity as a function of time during storage or operation. The term “storage” herein means to indicate the fact of products or compositions being stored from the time of being manufactured to the point in time of being used in final application. Retention of enzymatic activity as a function of time during storage may be called “storage stability”.

“Being used in final application” includes the act of putting a composition to a particular use or purpose. The particular purpose in the context of detergent compositions includes the ability to clean soiled material. In one embodiment, detergent compositions comprising enzymes have the ability to remove enzyme-sensitive stains.

Non-limiting examples of enzyme-sensitive stains include protease-sensitive stains (may also called proteinaceous stains herein), lipase-sensitive stains, amylase-sensitive stains, and cellulase sensitive stains. In one embodiment, enzyme-sensitive stains are removed by compositions comprising the respective enzyme or by detergent compositions comprising such compositions.

To determine and quantify changes in catalytic activity of enzymes stored or used under certain conditions over time, the “initial enzymatic activity” is measured under defined conditions at time zero (100%) before storage and at a certain point in time later (x %) after storage. By comparison of the values measured, a potential loss of enzymatic activity can be determined in its extent due to the process of storage. The extent of enzymatic activity loss determines an enzymes storage stability.

To be more precise, to determine and quantify changes in catalytic activity of enzymes stored or used under certain conditions over time, the “initial enzymatic activity” is measured under defined conditions at time zero before storage (i.e 100% enzymatic activity) and at a certain point in time later after storage (x % enzymatic activity). By comparison of the values measured, a potential loss of enzymatic activity can be determined in its extent due to the process of storage. The extent of enzymatic activity loss (100%-x % enzymatic activity) determines an enzymes storage stability. Storage stability may be called “good” (if the enzymatic activity loss during storage is insignificant) or “not good” (if the enzymatic activity loss during storage is significant). Significance is determined by the requirements of the final application.

“Half-life of enzymatic activity” is a measure for time required for the decaying of enzymatic activity to fall to one half (50%) of its initial value.

Parameters influencing the enzymatic activity of an enzyme and/or storage stability and/or operational stability are for example pH, temperature, and presence of oxidative substances:
“pH stability”, which refers to the ability of a protein to function at a particular pH. In general, most enzymes are working under conditions with rather high or rather low pHs. A substantial change in pH stability is evidenced by at least about 5% or greater modification (increase or decrease) in the half-life of the enzymatic activity, as compared to the enzymatic activity at the enzyme's optimum pH.
“thermal stability” or “thermostability” refer to the ability of a protein to function at a particular temperature. In general, most enzymes have a finite range of temperatures at which they function. In addition to enzymes that work in mid-range temperatures (e.g., room temperature), there are enzymes that are capable of working in very high or very low temperatures. A substantial change in thermal stability is evidenced by at least about 5% or greater modification (increase or decrease) in the half-life of the enzymatic activity when exposed to given temperature.
“oxidative stability”, which refers to the ability of a protein to function under oxidative conditions, in particular in the presence of various concentrations of H202, peracids and other oxidants. A substantial change in oxidative stability is evidenced by at least about a 5% or greater modification (increase or decrease) in the half-life of the enzymatic activity, as compared to the enzymatic activity present in the absence of oxidative compounds.
“stability to proteolysis” refers to the ability of a protein to withstand proteolysis. Enzymatically, proteolysis is catalyzed by proteases, enzymes which have proteolytic activity. Non-enzymatically induced proteolysis can be caused by extremes of pH and/or high temperatures. Stability to proteolysis herein includes stabilization of proteases to avoid self-proteolysis of proteases.

Enzymes storage stability normally is impaired in aqueous solution in the course of time. This can be avoided by storage of enzymes under non-hydrous conditions. Where non-hydrous conditions are not applicable, e.g. in compositions naturally comprising water, different or additional strategies need to be applied. Stabilization of proteolytic enzymes (proteases) by inhibition is a common technique to prevent proteolytic degradation (proteolysis) of proteins (such as enzymes) into peptides or amino acids (which may inactivate the functionality of e.g. an enzyme). Stabilization of proteases commonly makes use of reversible inhibition of the enzyme.

“Enzyme inhibitors” slow down the enzymatic activity by several mechanism as outlined below. Inhibitor binding is either reversible or irreversible. Irreversible inhibitors usually bind covalently to an enzyme by modifying the key amino acids necessary for enzymatic activity. Reversible inhibitors usually bind non-covalently (hydrogen bonds, hydrophobic interactions, ionic bonds). Four general kinds of reversible inhibitors are known:

(1) substrate and inhibitor compete for access to the enzymes active site (competitive inhibition),

(2) inhibitor binds to substrate-enzyme complex (non-competitive inhibition),

(3) binding of inhibitor reduces enzymatic activity but does not affect binding of substrate (non-competitive inhibition),

(4) inhibitor can bind to enzyme at the same time as substrate (mixed inhibition).

By using enzyme inhibitors, an enzyme is assumed to be stabilized. “Stabilized enzyme” in the context of the invention is the effect resulting from temporarily inhibiting an enzyme (reversible inhibition of the same) in its catalytic activity when compared to the catalytic activity of the same, non-inhibited enzyme. In one embodiment of the present invention, a protease is inhibited in its proteolytic activity by a reversible inhibitor comprised in a composition of the invention. Due to inhibition of proteolytic activity of at least one protease, another enzyme and the protease itself may be stabilized as their proteolytic degradation may be prevented resulting in retention of the catalytic activity of the other enzyme.

“Increased stability” or “improved stability” according to the current invention relates to the effect resulting from temporary inhibition of the catalytic activity of an enzyme when compared to the catalytic activity of the same, non-inhibited enzyme.

“Increased stability” may mean “increased storage stability” and “improved stability” may mean “improved storage stability”.

In one embodiment, the stability of a protease is increased or improved when the stabilized protease retains its catalytic activity after storage when compared to the same, non-stabilized protease before storage.

Additionally, an enzyme which is not a protease has increased or improved stability in the context of the current invention when said enzyme retains its catalytic activity in the presence of a stabilized protease, when compared to the same enzyme in the presence of a non-stabilized protease.

Enzymes to be stabilized according to the invention are hydrolases classified under EC 3 and other enzymes. EC-numbers are those according to the Nomenclature of the International Union of Biochemistry and Molecular Biology and preferably relate to the corresponding versions as valid as of Jan. 1, 2016.

“Hydrolases” of class EC 3 are acting on ester bonds (EC 3.1, e.g. lipase), sugars (EC 3.2, e.g. amylase, cellulase), ether bonds (EC 3.3), peptide bonds (EC 3.4, e.g. protease), carbon-nitrogen bonds (EC 3.5), acid anhydrates (EC 3.6), carbon-carbon bonds (EC 3.7), halide bonds (EC 3.8), phosphorus-nitrogen bonds (EC 3.9), Sulphur-nitrogen bonds (EC 3.10), carbon-phosphorus bonds (EC 3.11), sulfur-sulfur bonds (EC 3.12), and carbon-sulfur bonds (EC 3.13).

A composition according to the invention comprises

component (a): at least one boron-containing compound and

component (b): pentane-1,2-diol and optionally one or more further diols,

wherein the composition is liquid at 20° C. and 101.3 kPa.

Component (a) within the invention means at least one boron-containing compound. Boron-containing compounds are selected from boric acid or its derivatives and from boronic acid or its derivatives such as aryl boronic acids or its derivatives, from salts thereof, and from mixtures thereof. Boric acid herein may be called orthoboric acid. In one embodiment, at least one compound comprised in component (a) is selected from the group consisting of benzene boronic acid (BBA) and derivatives thereof. Preferably, component (a) is selected from the group consisting of benzene boronic acid (BBA) which may be called phenyl boronic acid (PBA) herein, derivatives thereof, and mixtures thereof.

In one embodiment, phenyl boronic acid derivatives are selected from the group consisting of the derivatives of formula (I) and (II) formula:

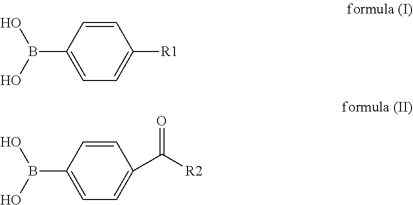

Wherein R1 is selected from the group consisting of hydrogen, hydroxy, non-substituted or substituted C₁-C₆ alkyl, and non-substituted or substituted C₁-C₆ alkenyl; in a preferred embodiment, R1 is selected from the group consisting of hydroxy, and non-substituted C₁ alkyl.

Wherein R2 is selected from the group consisting of hydrogen, hydroxy, non-substituted or substituted C₁-C₆ alkyl, and non-substituted or substituted C₁-C₆ alkenyl; in a preferred embodiment, R2 is selected from the group consisting of H, hydroxy, and substituted C₁ alkyl.

In one embodiment, phenyl-boronic acid derivatives are selected from the group consisting of 4-formyl phenyl boronic acid (4-FPBA), 4-carboxy phenyl boronic acid (4-CPBA), 4-(hydroxymethyl) phenyl boronic acid (4-HMPBA), and p-tolylboronic acid (p-TBA).

In one embodiment, at least one compound comprised in component (a) is selected from the group consisting of benzene boronic acid (BBA) and 4-formyl phenyl boronic acid (4-FPBA). In a preferred embodiment, component (a) is selected from the group consisting of benzene boronic acid (BBA) and 4-formyl phenyl boronic acid (4-FPBA).

Other suitable derivatives include 2-thienyl boronic acid, 3-thienyl boronic acid, (2-acetamidophenyl) boronic acid, 2-benzofuranyl boronic acid, 1-naphthyl boronic acid, 2-naphthyl boronic acid, 2-FPBA, 3-FBPA, 1-thianthrenyl boronic acid, 4-dibenzofuran boronic acid, 5-methyl-2-thienyl boronic acid, 1-benzothiophene-2 boronic acid, 2-furanyl boronic acid, 3-furanyl boronic acid, 4,4 biphenyl-diboronic acid, 6-hydroxy-2-naphthaleneboronic acid, 4-(methylthio) phenyl boronic acid, 4-(trimethylsilyl) phenyl boronic acid, 3-bromothiophene boronic acid, 4-methylthiophene boronic acid, 2-naphthyl boronic acid, 5-bromothiophene boronic acid, 5-chlorothiophene boronic acid, dimethylthiophene boronic acid, 2-bromophenyl boronic acid, 3-chlorophenyl boronic acid, 3-methoxy-2-thiophene boronic acid, p-methyl-phenylethyl boronic acid, 2-thianthrenyl boronic acid, di-benzothiophene boronic acid, 9-anthracene boronic acid, 3,5 dichlorophenyl boronic, acid, diphenyl boronic acid anhydride, o-chlorophenyl boronic acid, p-chlorophenyl boronic acid, m-bromophenyl boronic acid, p-bromophenyl boronic acid, p-fluorophenyl boronic acid, octyl boronic acid, 1,3,5 trimethylphenyl boronic acid, 3-chloro-4-fluorophenyl boronic acid, 3-aminophenyl boronic acid, 3,5-bis-(trifluoromethyl) phenyl boronic acid, 2,4 dichlorophenyl boronic acid, and 4-methoxyphenyl boronic acid.

Component (b) comprises at least pentane-1,2-diol and optionally one or more further diols. In one embodiment, pentane-1,2-diol is mixed of with other water-miscible alcohols. Such other water-miscible alcohols may be selected from the group consisting of ethane-1,2-diol, propane-1,2-diol, butane-1,2-diol, propane-1,2,3-triol, 2-(2-hydroxyethoxy)ethan-1-ol, 2-(2-hydroxypropoxy)propan-1-ol, and mixtures thereof.

In one embodiment, pentane-1,2-diol is mixed with other alcohols containing a vicinal diol selected from the group consisting of ethane-1,2-diol, propane-1,2-diol, butane-1,2-diol or propane-1,2,3-triol. In a preferred embodiment, component (b) is a mixture of propane-1,2-diol and pentane-1,2-diol or a mixture of propane-1,2,3-triol and pentane-1,2-diol.

In one embodiment, the composition comprising components (a) and (b) as described above, comprises an additional component (c), wherein component (c) comprises at least one protease and optionally one or more further enzymes. A composition comprising component (a), component (b) and component (c) may be called “enzyme stabilizing composition” herein.

Any protease comprised in component (c) is a member of EC class 3.4. “Proteases” of class EC 3.4 are further classified as aminopeptidases (EC 3.4.11), dipeptidases (EC 3.4.13), dipeptidylpeptidases and tripeptidyl-peptidases (EC 3.4.14), peptidyl-dipeptidases (EC 3.4.15), serine-type carboxypeptidases (EC 3.4.16), metallocarboxypeptidases (EC 3.4.17), cysteine-type carboxypeptidases (EC 3.4.18), omega peptidases (EC 3.4.19), serine endopeptidases (EC 3.4.21), cysteine endopeptidases (EC 3.4.22), aspartic endopeptidases (EC 3.4.23), metallo-endopeptidases (EC 3.4.24), threonine endopeptidases (EC 3.4.25), endopeptidases of unknown catalytic mechanism (EC 3.4.99).

In one embodiment, at least one enzyme comprised in component (c) is selected from the group of serine proteases (EC 3.4.21). In one embodiment component (c) comprises more than one serine proteases. In one embodiment, “one or more further enzymes” comprised in component (c) are selected from one or more proteases other than serine proteases, and/or “one or more enzymes other than proteases”, such as lipases, amylases, and cellulases.

Serine proteases or serine peptidases are characterized by having a serine in the catalytically active site, which forms a covalent adduct with the substrate during the catalytic reaction.

A serine protease according to the invention may be selected from the group consisting of chymotrypsin (e.g., EC 3.4.21.1), elastase (e.g., EC 3.4.21.36), elastase (e.g., EC 3.4.21.37 or EC 3.4.21.71), granzyme (e.g., EC 3.4.21.78 or EC 3.4.21.79), kallikrein (e.g., EC 3.4.21.34, EC 3.4.21.35, EC 3.4.21.118, or EC 3.4.21.119,) plasmin (e.g., EC 3.4.21.7), trypsin (e.g., EC 3.4.21.4), thrombin (e.g., EC 3.4.21.5,) and subtilisin (also known as subtilopeptidase, e.g., EC 3.4.21.62), the latter hereinafter also being referred to as “subtilisin”. Preferably, at least one enzyme of component (c) is selected from subtilisins (also called subtilisin proteases or subtilases).

Crystallographic structures of proteases show that the active site is commonly located in a groove on the surface of the molecule between adjacent structural domains, and the substrate specificity is dictated by the properties of binding sites arranged along the groove on one or both sides of the catalytic site that is responsible for hydrolysis of the scissile bond. Accordingly, the specificity of a protease can be described by use of a conceptual model in which each specificity subsite is able to accommodate the sidechain of a single amino acid residue. The sites are numbered from the catalytic site, S1, S2 . . . Sn towards the N-terminus of the substrate, and S1′, S2′ . . . Sn′ towards the C-terminus. The residues they accommodate are numbered P1, P2 . . . Pn, and P1′, P2′ . . . Pn′, respectively:

| | Substrate | | | | | | |
|---|---|---|---|---|---|---|---|
| | P3 | P2 | P1 | + | P1′ | P2′ | P3′ |
| Enzyme | S3 | S2 | S1 | * | S1′ | S2′ | S3′ |

In this representation the catalytic site of the enzyme is marked “*” and the peptide bond cleaved (the scissile bond) is indicated by the symbol “+”.

In general, the three main types of protease activity (proteolytic activity) are: trypsin-like, where there is cleavage of amide substrates following Arg (N) or Lys (K) at P1, chymotrypsin-like where cleavage occurs following one of the hydrophobic amino acids at P1, and elastase-like with cleavage following an Ala (A) at P1.

A sub-group of the serine proteases tentatively designated subtilases has been proposed by Siezen et al. (1991), Protein Eng. 4:719-737 and Siezen et al. (1997), Protein Science 6:501-523. They are defined by homology analysis of more than 170 amino acid sequences of serine proteases previously referred to as subtilisin-like proteases. A subtilisin was previously often defined as a serine protease produced by Gram-positive bacteria or fungi, and according to Siezen et al. now is a subgroup of the subtilases. A wide variety of subtilases have been identified, and the amino acid sequence of a number of subtilases has been determined. For a more detailed description of such subtilases and their amino acid sequences reference is made to Siezen et al. (1997), Protein Science 6:501-523.

The subtilases may be divided into 6 sub-divisions, i.e. the subtilisin family, thermitase family, the proteinase K family, the lantibiotic peptidase family, the kexin family and the pyrolysin family.

A subgroup of the subtilases are the subtilisins which are serine proteases from the family S8 as defined by the MEROPS database (http://merops.sanger.ac.uk). Peptidase family S8 contains the serine endopeptidase subtilisin and its homologues. In subfamily S8A, the active site residues frequently occur in the motifs Asp-Thr/Ser-Gly (which is similar to the sequence motif in families of aspartic endopeptidases in clan AA), His-Gly-Thr-His and Gly-Thr-Ser-Met-Ala-Xaa-Pro. Most members of the family are active at neutral-mildly alkali pH. Many peptidases in the family are thermostable. Casein is often used as a protein substrate and a typical synthetic substrate is Suc-Ala-Ala-Pro-Phe-NHPhNO₂.

Prominent members of family S8, subfamily A are:

| Name | MEROPS Family S8, Subfamily A |
|---|---|
| Subtilisin Carlsberg | S08.001 |
| Subtilisin lentus | S08.003 |
| Thermitase | S08.007 |
| Subtilisin BPN′ | S08.034 |
| Subtilisin DY | S08.037 |
| Alkaline peptidase | S08.038 |
| Subtilisin ALP 1 | S08.045 |
| Subtilisin sendai | S08.098 |
| Alkaline elastase YaB | S08.157 |

The subtilisin related class of serine proteases share a common amino acid sequence defining a catalytic triad which distinguishes them from the chymotrypsin related class of serine proteases. Subtilisins and chymotrypsin related serine proteases both have a catalytic triad comprising aspartate, histidine and serine.

In the subtilisin related proteases the relative order of these amino acids, reading from the amino to carboxy-terminus is aspartate-histidine-serine. In the chymotrypsin related proteases the relative order, however is histidine-aspartate-serine. Thus, subtilisin herein refers to a serine protease having the catalytic triad of subtilisin related proteases. Examples include the subtilisins as described in WO 89/06276 and EP 0283075, WO 89/06279, WO 89/09830, WO 89/09819, WO 91/06637 and WO 91/02792.

Parent proteases of the subtilisin type (EC 3.4.21.62) and variants may be bacterial proteases. Said bacterial protease may be a Gram-positive bacterial polypeptide such as a Bacillus, Clostridium, Enterococcus, Geobacillus, Lactobacillus, Lactococcus, Oceanobacillus, Staphylococcus, Streptococcus, or Streptomyces protease, or a Gram-negative bacterial polypeptide such as a Campylobacter, E. coli, Flavobacterium, Fusobacterium, Helicobacter, Ilyobacter, Neisseria, Pseudomonas, Salmonella or Ureaplasma protease. They act as unspecific endopeptidases, i.e. they hydrolyze any peptide bonds. Their pH optimum is usually within the neutral to distinctly alkaline range. A review of this family is provided, for example, in “Subtilases: Subtilisin-like Proteases” by R. Siezen, pages 75-95 in “Subtilisin enzymes”, edited by R. Bott and C. Betzel, New York, 1996.

Commercially available protease enzymes include but are not limited to those sold under the trade names Alcalase®, Blaze®, Duralase™, Durazym™, Relase®, Relase® Ultra, Savinase®, Savinase® Ultra, Primase®, Polarzyme®, Kannase®, Liquanase®, Liquanase® Ultra, Ovozyme®, Coronase®, Coronase® Ultra, Neutrase®, Everlase® and Esperase® (Novozymes A/S), those sold under the tradename Maxatase®, Maxacal®, Maxapem®, Purafect®, Purafect® Prime, Purafect MA®, Purafect Ox®, Purafect OxP®, Puramax®, Properase®, FN2®, FN3®, FN4®, Excellase®, Eraser®, Ultimase®, Opticlean®, Effectenz®, Preferenz® and Optimase® (Danisco/DuPont), Axapem™ (Gist-Brocases N.V.), Bacillus lentus Alkaline Protease (BLAP; sequence shown in FIG. 29 of U.S. Pat. No. 5,352,604) and variants thereof, and KAP (Bacillus alkalophllus subtilisin) from Kao.

In one aspect of the invention, the serine proteases (parent and/or variants) may be a Bacillus alcalophilus, Bacillus amyloliquefaciens, Bacillus brevis, Bacillus circulars, Bacillus clausii, Bacillus coagulans, Bacillus firmus, Bacillus gibsonii, Bacillus lautus, Bacillus lentus, Bacillus licheniformis, Bacillus megaterium, Bacillus pumilus, Bacillus sphaericus, Bacillus stearothermophilus, Bacillus subtilis, or Bacillus thuringiensis protease.

In one embodiment of the present invention, the subtilase is selected from the following:
subtilase from Bacillus amyloliquefaciens BPN′ (described by Vasantha et al. (1984) J. Bacteriol. Volume 159, p. 811-819 and J A Wells et al. (1983) in Nucleic Acids Research, Volume 11, p. 7911-7925),
subtilase from Bacillus licheniformis (subtilisin Carlsberg; disclosed in E L Smith et al. (1968) in J. Biol Chem, Volume 243, pp. 2184-2191, and Jacobs et al. (1985) in Nucl. Acids Res, Vol 13, p. 8913-8926),
subtilase PB92 (original sequence of the alkaline protease PB92 is described in EP 283075 A2),
subtilase 147 and/or 309 (Esperase®, Savinase®) as disclosed in GB 1243784,
subtilase from Bacillus lentus as disclosed in WO 91/02792, such as from Bacillus lentus DSM 5483 or the variants of Bacillus lentus DSM 5483 as described in WO 95/23221,
subtilase from Bacillus alcalophllus (DSM 11233) disclosed in DE 10064983,
subtilase from Bacillus gibsomii (DSM 14391) as disclosed in WO 2003/054184,
subtilase from Bacillus sp. (DSM 14390) disclosed in WO 2003/056017,
subtilase from Bacillus sp. (DSM 14392) disclosed in WO 2003/055974,
subtilase from Bacillus gibsonii (DSM 14393) disclosed in WO 2003/054184,
subtilase having SEQ ID NO: 4 as described in WO 2005/063974 or a subtilisin which is at least 40% identical thereto and having proteolytic activity,
subtilase having SEQ ID NO: 4 as described in WO 2005/103244 or subtilisin which is at least 80% identical thereto and having proteolytic activity,
subtilase having SEQ ID NO: 7 as described in WO 2005/103244 or subtilisin which is at least 80% identical thereto and having proteolytic activity, and
subtilase having SEQ ID NO: 2 as described in application DE 102005028295.4 or subtilisin which is this at least 66% identical thereto and having proteolytic activity.

Examples of useful subtilisin proteases in accordance with the present invention comprise the variants described in: WO 92/19729, WO 95/23221, WO 96/34946, WO 98/20115, WO 98/20116, WO 99/11768, WO 01/44452, WO 02/088340, WO 03/006602, WO 2004/03186, WO 2004/041979, WO 2007/006305, WO 2011/036263, WO 2011/036264, and WO 2011/072099.

Suitable examples comprise especially protease variants of subtilisin protease derived from SEQ ID NO:22 as described in EP 1921147 (which is the sequence of mature alkaline protease from Bacillus lentus DSM 5483) with amino acid substitutions in one or more of the following positions: 3, 4, 9, 15, 24, 27, 33, 36, 57, 68, 76, 77, 87, 95, 96, 97, 98, 99, 100, 101, 102, 103, 104, 106, 118, 120, 123, 128, 129, 130, 131, 154, 160, 167, 170, 194, 195, 199, 205, 206, 217, 218, 222, 224, 232, 235, 236, 245, 248, 252 and 274 (according to the BPN′ numbering), which have proteolytic activity. In one embodiment, such a subtilisin protease is not mutated at positions Asp32, His64 and Ser221 (according to BPN′ numbering).

In one embodiment, the subtilisin has SEQ ID NO:22 as described in EP 1921147, or a subtilisin which is at least 80% identical thereto and has proteolytic activity. In one embodiment, a subtilisin is at least 80% identical to SEQ ID NO:22 as described in EP 1921147 and is characterized by having amino acid glutamic acid (E), or aspartic acid (D), or asparagine (N), or glutamine (Q), or alanine (A), or glycine (G), or serine (S) at position 101 (according to BPN′ numbering) and has proteolytic activity. In one embodiment, subtilisin is at least 80% identical to SEQ ID NO:22 as described in EP 1921147 and is characterized by having amino acid glutamic acid (E), or aspartic acid (D), at position 101 (according to BPN′ numbering) and has proteolytic activity. Such a subtilisin variant may preferably comprise an amino acid substitution at position 101, such as R101E or R101D, alone or in combination with one or more substitutions at positions 3, 4, 9, 15, 24, 27, 33, 36, 57, 68, 76, 77, 87, 95, 96, 97, 98, 99, 100, 101, 102, 103, 104, 106, 118, 120, 123, 128, 129, 130, 131, 154, 160, 167, 170, 194, 195, 199, 205, 206, 217, 218, 222, 224, 232, 235, 236, 245, 248, 252 and/or 274 (according to BPN′ numbering) and has proteolytic activity.

In another embodiment, a subtilisin is at least 80% identical to SEQ ID NO:22 as described in EP 1921147 and is characterized by comprising at least the following amino acids (according to BPN′ numbering) and has proteolytic activity:
(a) threonine at position 3 (3T)
(b) isoleucine at position 4 (41)
(c) alanine, threonine or arginine at position 63 (63A, 63T, or 63R)
(d) aspartic acid or glutamic acid at position 156 (156D or 156E)
(e) proline at position 194 (194P)
(f) methionine at position 199 (199M)
(g) isoleucine at position 205 (2051)
(h) aspartic acid, glutamic acid or glycine at position 217 (217D, 217E or 217G),
(i) combinations of two or more amino acids according to (a) to (h).

In another embodiment, a subtilisin is at least 80% identical to SEQ ID NO:22 as described in EP 1921147 and is characterized by comprising one amino acid (according to (a)-(h)) or combinations according to (i) together with the amino acid 101E, 101D, 101N, 101Q, 101A, 101G, or 101S (according to BPN′ numbering) and has proteolytic activity.

In one embodiment, subtilisin is at least 80% identical to SEQ ID NO:22 as described in EP 1921147 and is characterized by comprising the mutation (according to BPN′ numbering) R101E, or S3T+V4I+V205I, or S3T+V4I+V199M+V205I+L217D, and has proteolytic activity.

In another embodiment, the subtilisin comprises an amino acid sequence having at least 80% identity to SEQ ID NO:22 as described in EP 1921147 and being further characterized by comprising R101E and S3T, V4I, and V205I (according to the BPN′ numbering) and has proteolytic activity.

In another embodiment, a subtilisin comprises an amino acid sequence having at least 80% identical to SEQ ID NO:22 as described in EP 1921147 and being further characterized by comprising R101 E, and one or more substitutions selected from the group consisting of S156D, L262E, Q137H, S3T, R45E,D,Q, P55N, T58W,Y,L, Q59D,M,N,T, G61 D,R, S87E, G97S, A98D,E,R, S106A,W, N117E, H120V,D,K,N, S125M, P129D, E136Q, S144W, S161T, S163A,G, Y171 L, A172S, N185Q, V199M, Y209W, M222Q, N238H, V244T, N261T,D and L262N,Q,D (as described in WO 2016/096711 and according to the BPN′ numbering) and has proteolytic activity.

%-identity for subtilisin variants is calculated as disclosed above. Subtilisin variant enzymes as disclosed above which are at least n % identical to the respective parent sequences include variants with n being at least 40 to 100. Depending on the %-identity values applicable as provided above, subtilisin variants in one embodiment have proteolytic activity and are at least 40%, at least 45%, at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98% or at least 99% identical when compared to the full length polypeptide sequence of the parent enzyme.

In another embodiment, the invention relates to subtilisin variants comprising conservative mutations not pertaining the functional domain of the respective subtilisin protease. Depending on the %-identity values applicable as provided above, subtilisin variants of this embodiment have proteolytic activity and are at least 40%, at least 45%, at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98% or at least 99% similar when compared to the full length polypeptide sequence of the parent enzyme.

In one embodiment, component (c) comprises at least one subtilisin protease selected from those which are at least 90% identical to SEQ ID No:2 of this invention and have proteolytic activity. Preferably, the subtilisin protease is an alkaline protease from Bacillus lentus.

In one embodiment, component (c) comprises at least one subtilisin protease selected from those which are at least 90% identical to SEQ ID No:1 of this invention and have proteolytic activity. Preferably, the subtilisin protease is an alkaline protease from Bacillus lentus.

Proteases, including serine proteases, according to the invention have “proteolytic activity” or “protease activity”. This property is related to hydrolytic activity of a protease (proteolysis, which means hydrolysis of peptide bonds linking amino acids together in a polypeptide chain) on protein containing substrates, e.g. casein, haemoglobin and BSA. Quantitatively, proteolytic activity is related to the rate of degradation of protein by a protease or proteolytic enzyme in a defined course of time. The methods for analyzing proteolytic activity are well-known in the literature (see e.g. Gupta et al. (2002), Appl. Microbiol. Biotechnol. 60: 381-395).

According to the invention, proteolytic activity as such can be determined by using Succinyl-Ala-Ala-Pro-Phe-p-nitroanilide (Suc-AAPF-pNA, short AAPF; see e.g. DelMar et al. (1979), Analytical Biochem 99, 316-320) as substrate pNA is cleaved from the substrate molecule by proteolytic cleavage, resulting in release of yellow color of free pNA which can be quantified by measuring OD₄₀₅. Other methods are known to those skilled in the art.

In one embodiment, component (c) comprises at least one serine protease in amounts in the range of 0.1 g/L to 150 g/L, 1 g/L to 100 g/L, 10 g/L to 100 g/L, or 30 g/L to 90 g/L.

In one embodiment of the invention, component (c) comprises one or more other enzyme(s) which are not proteases, which may be called “other enzymes” herein. “Other enzymes” according to the invention may be selected from any enzymes suitable for the application of compositions of the invention such as lipase, amylase, cellulase, lyases, peroxidases, oxidases perhydrolases, mannanases, pectinase, arabinase, galactanase, xylanase.

In one embodiment, the composition of the invention comprises at least one lipase. “Lipases”, “lipolytic enzyme”, “lipid esterase”, all refer to an enzyme of EC class 3.1.1 (“carboxylic ester hydrolase”). Such an enzyme may have lipase activity (or lipolytic activity; triacylglycerol lipase, EC 3.1.1.3), cutinase activity (EC 3.1.1.74; enzymes having cutinase activity may be called cutinase herein), sterol esterase activity (EC 3.1.1.13) and/or wax-ester hydrolase activity (EC 3.1.1.50). Lipases include those of bacterial or fungal origin.

Commercially available lipase enzymes include but are not limited to those sold under the trade names Lipolase™, Lipex™, Lipolex™ and Lipoclean™ (Novozymes NS), Lumafast (originally from Genencor) and Lipomax (Gist-Brocades/now DSM).

In one aspect of the invention, a suitable lipase is selected from the following:
lipases from Humicola (synonym Thermomyces), e.g. from H. lanuginosa (T. lanuginosus) as described in EP 258068, EP 305216, WO 92/05249 and WO 2009/109500 or from H. insolens as described in WO 96/13580,
lipases derived from Rhizomucor miehei as described in WO 92/05249.
lipase from strains of Pseudomonas (some of these now renamed to Burkholderia), e.g. from P. alcaligenes or P. pseudoalcaligenes (EP 218272, WO 94/25578, WO 95/30744, WO 95/35381, WO 96/00292), P. cepacia (EP 331376), P. stutzeri (GB 1372034), P. fluorescens, Pseudomonas sp. strain SD705 (WO 95/06720 and WO 96/27002), P. wisconsinensis (WO 96/12012), Pseudomonas mendocina (WO 95/14783), P. glumae (WO 95/35381, WO 96/00292)
lipase from Streptomyces griseus (WO 2011/150157) and S. pristinaespiralis (WO 2012/137147), GDSL-type Streptomyces lipases (WO 2010/065455),
lipase from Thermobifida fusca as disclosed in WO 2011/084412,
lipase from Geobacillus stearothermophilus as disclosed in WO 2011/084417,
Bacillus lipases, e.g. as disclosed in WO 00/60063, lipases from B. subtilis as disclosed in Dartois et al. (1992), Biochemica et Biophysica Acta, 1131, 253-360 or WO 2011/084599, B. stearothermophllus (JP S64-074992) or B. pumllus (WO 91/16422).
Lipase from Candida antarctica as disclosed in WO 94/01541.
Suitable lipases include also those referred to as acyltransferases or perhydrolases, e.g. acyltransferases with homology to Candida antarctica lipase A (WO 2010/111143), acyltransferase from Mycobacterium smegmatis (WO 2005/056782), perhydrolases from the CE7 family (WO 2009/67279), and variants of the M. smegmatis perhydrolase in particular the S54V variant (WO 2010/100028).

In one aspect of the invention, a suitable cutinase is selected from the following:
cutinase from Pseudomonas mendocina (U.S. Pat. No. 5,389,536, WO 88/09367)
cutinase from Magnaporthe grisea (WO 2010/107560),
cutinase from Fusarum solani pisi as disclosed in WO 90/09446, WO 00/34450 and WO 01/92502
cutinase from Humicola lanuginosa as disclosed in WO 00/34450 and WO 01/92502

Suitable lipases and/or cutinases include also those which are variants of the above described lipases and/or cutinases which have lipolytic activity or cutinase activity. Such suitable lipase variants are e.g. those which are developed by methods as disclosed in WO 95/22615, WO 97/04079, WO 97/07202, WO 00/60063, WO 2007/087508, EP 407225 and EP 260105. Suitable lipases/cutinases include also those, which are variants of the above described lipases/cutinases which have lipolytic activity or cutinase activity. Suitable lipase/cutinase variants include variants with at least 40 to 100% identity when compared to the full length polypeptide sequence of the parent enzyme as disclosed above. In one embodiment, lipase/cutinase variants having lipolytic activity or cutinase activity may be at least 40%, at least 45%, at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98% or at least 99% identical when compared to the full length polypeptide sequence of the parent enzyme as disclosed above.

In another embodiment, the invention relates to lipase/cutinase variants comprising conservative mutations not pertaining the functional domain of the respective lipase/cutinase. Lipase/cutinase variants of this embodiment having lipolytic activity or cutinase activity may be at least 40%, at least 45%, at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98% or at least 99% similar when compared to the full length polypeptide sequence of the parent enzyme.

Lipases according to the invention have “lipolytic activity”. The methods for determining lipolytic activity are well-known in the literature (see e.g. Gupta et al. (2003), Biotechnol. Appl. Biochem. 37, p. 63-71).

In one embodiment, the composition of the invention comprises at least one amylase. “Amylases” according to the invention (alpha and/or beta) include those of bacterial or fungal origin (EC 3.2.1.1 and 3.2.1.2, respectively). Chemically modified or protein engineered mutants are included.

Commercially available amylase enzymes include but are not limited to those sold under the trade names Duramyl™, Termamyl™, Fungamyl™, Stainzyme™, Stainzyme Plus™, Natalase™, Liquozyme X and BAN™ (from Novozymes NS), and Rapidase™, Purastar™, Powerase™, Effectenz™ (M100 from DuPont), Preferenz™ (S1000, S110 and F1000; from DuPont), PrimaGreen™ (ALL; DuPont), Optisize™ (DuPont).

In one aspect of the present invention, the amylase is a parent or variant enzyme which is selected from the following:
amylases from Bacillus licheniformis having SEQ ID NO:2 as described in WO 95/10603. Suitable variants are those which are at least 90% identical to SEQ ID NO: 2 as described in WO 95/10603 and/or comprising one or more substitutions in the following positions: 15, 23, 105, 106, 124, 128, 133, 154, 156, 178, 179, 181, 188, 190, 197, 201, 202, 207, 208, 209, 211, 243, 264, 304, 305, 391, 408, and 444 which have amylolytic activity. Such variants are described in WO 94/02597, WO 94/018314, WO 97/043424 and SEQ ID NO:4 of WO 99/019467.
amylases from B. stearothermophllus having SEQ ID NO:6 as disclosed in WO 02/10355 or an amylase which is at least 90% identical thereto having amylolytic activity. Suitable variants of SEQ ID NO:6 include those which is at least 90% identical thereto and/or further comprise a deletion in positions 181 and/or 182 and/or a substitution in position 193.
amylases from Bacillus sp. 707 having SEQ ID NO:6 as disclosed in WO 99/19467 or an amylase which is at least 90% identical thereto having amylolytic activity.
amylases from Bacillus halmapalus having SEQ ID NO:2 or SEQ ID NO:7 as described in WO 96/23872, also described as SP-722, or an amylase which is at least 90% identical to one of the sequences which has amylolytic activity.
amylases from Bacillus sp. DSM 12649 having SEQ ID NO:4 as disclosed in WO 00/22103 or an amylase which is at least 90% identical thereto having amylolytic activity.
amylases from Bacillus strain TS-23 having SEQ ID NO:2 as disclosed in WO 2009/061380 or an amylase which is at least 90% identical thereto having amylolytic activity.
amylases from Cytophaga sp. having SEQ ID NO:1 as disclosed in WO 2013/184577 or an amylase which is at least 90% identical thereto having amylolytic activity.
amylases from Bacillus megaterium DSM 90 having SEQ ID NO:1 as disclosed in WO 2010/104675 or an amylase which is at least 90% identical thereto having amylolytic activity.

Suitable amylases are comprising amino acids 1 to 485 of SEQ ID NO:2 as described in WO 00/60060 or amylases comprising an amino acid sequence which is at least 96% identical with amino acids 1 to 485 of SEQ ID NO:2 which have amylolytic activity.

Other suitable amylases are those having SEQ ID NO: 12 as described in WO 2006/002643 or amylases having at least 80% identity thereto and have amylolytic activity. Suitable amylases include those having at least 80% identity compared to SEQ ID NO:12 and/or comprising the substitutions at positions Y295F and M202LITV and have amylolytic activity.

Suitable amylases include those having SEQ ID NO:6 as described in WO 2011/098531 or amylases having at least 80% identity thereto having amylolytic activity. Suitable amylases include those having at least 80% identity compared to SEQ ID NO:6 and/or comprising a substitution at one or more positions selected from the group consisting of 193 [G,A,S,T or M], 195 [F,W,Y,L,I or V], 197 [F,W,Y,L,I or V], 198 [Q or N], 200 [F,W,Y,L,I or V], 203 [F,W,Y,L,I or V], 206 [F,W,Y,N,L,I,V,H,Q,D or E], 210 [F,W,Y,L,I or V], 212 [F,W,Y,L,I or V], 213 [G,A,S,T or M] and 243 [F,W,Y,L,I or V] and have amylolytic activity.

Suitable amylases are those having SEQ ID NO:1 as described in WO 2013/001078 or amylases having at least 85% identity thereto having amylolytic activity. Suitable amylases include those having at least 85% identity compared to SEQ ID NO:1 and/or comprising an alteration at two or more (several) positions corresponding to positions G304, W140, W189, D134, E260, F262, W284, W347, W439, W469, G476, and G477 and having amylolytic activity.

Further suitable amylases are those having SEQ ID NO:2 as described in WO 2013/001087 or amylases having at least 85% identity thereto and having amylolytic activity. Suitable amylases include those having at least 85% identity compared to SEQ ID NO:2 and/or comprising a deletion of positions 181+182, or 182+183, or 183+184, which have amylolytic activity. Suitable amylases include those having at least 85% identity compared to SEQ ID NO:2 and/or comprising a deletion of positions 181+182, or 182+183, or 183+184, which comprise one or two or more modifications in any of positions corresponding to W140, W159, W167, Q169, W189, E194, N260, F262, W284, F289, G304, G305, R320, W347, W439, W469, G476 and G477 and have amylolytic activity.

Amylases also include hybrid α-amylase from above mentioned amylases as for example as described in WO 2006/066594.

Suitable amylases include also those which are variants of the above described amylases which have amylolytic activity.

Depending on the %-identity values applicable as provided above, amylase variants in one embodiment may be those which are least 40 to 100% identical when compared to the full length polypeptide sequence of the parent enzyme as disclosed above. In one embodiment amylase variants having amylolytic activity may be at least 70%, at least 75%, at least 80%, at least 85%, at least 90%, at least 91%, at least 92%, at least 93%, at least 94%, at least 95%, at least 96%, at least 97%, at least 98% or at least 99% identical when compared to the full length polypeptide sequence of the parent enzyme as disclosed above.

In another embodiment, the invention relates to amylase variants comprising conservative mutations not pertaining the functional domain of the respective amylase. Depending on the %-identity values applicable as provided above, amylase variants in this embodiment may be amylases have amylolytic activity which may be least 70%, at least 75%, at least 80%, at least 85%, at least 90%, at least 91%, at least 92%, at least 93%, at least 94%, at least 95%, at least 96%, at least 97%, at least 98% or at least 99% similar when compared to the full length polypeptide sequence of the parent enzyme.

Amylases according to the invention have “amylolytic activity” or “amylase activity” according to the invention involves (endo)hydrolysis of glucosidic linkages in polysaccharides. α-amylase activity may be determined by assays for measurement of α-amylase activity which are known to those skilled in the art. Examples for assays measuring α-amylase activity are: α-amylase activity can be determined by a method employing Phadebas tablets as substrate (Phadebas Amylase Test, supplied by Magle Life Science) Starch is hydrolyzed by the aamylase giving soluble blue fragments. The absorbance of the resulting blue solution, measured spectrophotometrically at 620 nm, is a function of the α-amylase activity. The measured absorbance is directly proportional to the specific activity (activity/mg of pure α-amylase protein) of the α-amylase in question under the given set of conditions.

α-amylase activity can also be determined by a method employing the Ethyliden-4-nitrophenyl-α-D-maltoheptaosid (EPS). D-maltoheptaoside is a blocked oligosaccharide which can be cleaved by an endo-amylase. Following the cleavage, the α-glucosidase included in the kit to digest the substrate to liberate a free PNP molecule which has a yellow color and thus can be measured by visible spectophotometry at 405 nm. Kits containing EPS substrate and α-glucosidase is manufactured by Roche Costum Biotech (cat. No. 10880078103). The slope of the time dependent absorption-curve is directly proportional to the specific activity (activity per mg enzyme) of the α-amylase in question under the given set of conditions.

In one embodiment, the composition of the invention comprises at least one cellulase. “Cellulases”, “cellulase enzymes” or “cellulolytic enzymes” are enzymes involved in hydrolysis of cellulose. Three major types of cellulases are known, namely cellobiohydrolase (1,4-P-D-glucan cellobiohydrolase, EC 3.2.1.91), endo-ss-1,4-glucanase (endo-1,4-P-D-glucan 4-glucanohydrolase, EC 3.2.1.4) and ss-glucosidase (EC 3.2.1.21).

In one aspect of the invention, the cellulase is an endoglucanase of EC class 3.2.1.4 which may be named endoglucanase, endo-1,4-ss-D-glucan 4-glucano hydrolase, endo-1,4-beta-glucanase, carboxymethyl cellulase, and beta-1,4-glucanase. Endoglucanases may be classified by amino acid sequence similarities (Henrissat, B. Accessed at UniProt 10/26/2011) under family 5 containing more than 20 endoglucanases of EC 3.2.1.4. Reference is also made to T.-M. Enveri, “Microbial Cellulases” in W. M. Fogarty, Microbial Enzymes and Biotechnology, Applied Science Publishers, p. 183-224 (1983); Methods in Enzymology, (1988) Vol. 160, p. 200-391 (edited by Wood, W. A. and Kellogg, S. T.); Béguin, P., “Molecular Biology of Cellulose Degradation”, Annu. Rev. Microbiol. (1990), Vol. 44, pp. 219248; Begun, P. and Aubert, J-P., “The biological degradation of cellulose”, FEMS Microbiology Reviews 13 (1994) p. 25-58; Henrissat, B., “Cellulases and their interaction with cellulose”, Cellulose (1994), Vol. 1, pp. 169-196.

Commercially available cellulases are Celluzyme™, Endolase™, Carezyme™, Cellusoft™, Renozyme™, Celluclean™ (from Novozymes NS), Ecostone™, Biotouch™, Econase™, Ecopulp™ (from AB Enzymes Finland), Clazinase™, and Puradax HA™, Genencor detergent cellulase L, IndiAge™ Neutra (from Genencor International Inc./DuPont), Revitalenz™ (2000 from DuPont), Primafast™ (DuPont) and KAC500™ (from Kao Corporation).

Cellulases according to the invention include those of bacterial or fungal origin.

Suitable parent and variant enzymes are selected from the genus:
Bacillus, such as Bacillus sp. CBS 670.93 and CBS 669.93
Melanocarpus, such as Melanocarpus albomyces as disclosed in WO 97/14804
Clostridium, e.g. Clostridium thermocellum
Humicola, such as Humicola insolens (DSM1800) as disclosed in EP 0495257, EP 0531315, EP 0531372, U.S. Pat. No. 4,435,307, U.S. Pat. No. 5,648,263, U.S. Pat. No. 5,776,757, WO 89/09259, WO 91/17244, WO 94/07998 (sequence displayed in FIG. 1 “43kdhumand variants thereof), WO 95/24471, WO 96/11262 and WO 98/12307.
Fusarium, such as Fusarium oxysporum e.g. strain J79 (DSM2672) as disclosed in EP 0495257, EP 0531315, EP 0531372, U.S. Pat. No. 5,648,263, U.S. Pat. No. 5,776,757, WO 89/09259, WO 91/17244, WO 95/24471 and WO 96/11262
Thielavia, such as Thielavia terrestris or Myceliophthora thermophila strain CBS 11765 as disclosed in EP 0531315, U.S. Pat. No. 5,648,263, U.S. Pat. No. 5,776,757, WO 89/09259, WO 91/17244, WO 95/24471, WO 96/11262, WO 96/29397 (SEQ ID NO: 9 and variants thereof), and WO 98/12307.
Trichoderma, such as Trichoderma reesei; Trichoderma longibrachiatum or Trichoderma harzianum as disclosed in EP 1305432, EP 1240525, WO 92/06165, WO 94/21801, WO 94/26880, WO 95/02043, WO 95/24471 and WO 02/099091.
Aspergillus, such as Aspergillus aculeatus as disclosed in WO 93/17244
Erwinia, such as Erwinia chrysanthermias described by M. H. Boyer et. al. in European Journal of Biochemistry, vol. 162, page 311-316 (1987).
Acremonium such as Acremonium sp., Acremonium persicinum, Acremonium acremonium, Acremonium brachypenium, Acremonium dichromosporum, Acremonium obclavatum, Acremonium pinkertoniae, Acremonium roseogriseum, Acremonium incoloratum, and Acremonium furatum as disclosed in WO 96/11262 and WO 96/29397 (SEQ ID NO: 5 and variants thereof).
Cellvibrio such as Cellvibrio mixtus DSM 11683, Cellvibrio mixtus DSM 11684, Cellvibrio mixtus DSM 11685, Cellvibrio mixtus ACM 2601, Cellvibrio mixtus DSM 1523, and Cellvibrio gilvus DSM 11686, as disclosed in WO 98/08940.
Cephalosporium, such as Cephalosporium sp. RYM-202 as disclosed in WO 96/11262.

Suitable cellulases include also those, which are variants of the above described cellulases which have cellulolytic activity. Suitable cellulase variants include variants with at least 40 to 100% identity when compared to the full length polypeptide sequence of the parent enzyme as disclosed above. In one embodiment cellulase variants having cellulolytic activity may be at least 70%, at least 75%, at least 80%, at least 85%, at least 90%, at least 91%, at least 92%, at least 93%, at least 94%, at least 95%, at least 96%, at least 97%, at least 98% or at least 99% identical when compared to the full length polypeptide sequence of the parent enzyme as disclosed above.

In another embodiment, the invention relates to cellulase variants comprising conservative mutations not pertaining the functional domain of the respective cellulase. Cellulase variants of this embodiment having cellulolytic activity may be at least 70%, at least 75%, at least 80%, at least 85%, at least 90%, at least 91%, at least 92%, at least 93%, at least 94%, at least 95%, at least 96%, at least 97%, at least 98% or at least 99% similar when compared to the full length polypeptide sequence of the parent enzyme.

Cellulases according to the invention have “cellulolytic activity” or “cellulase activity” according to the invention involves endoglucanase activity. Assays for measurement of endoglucanase activity are known to those skilled in the art.

For example, cellulolytic activity may be determined by virtue of the fact that cellulase hydrolyses carboxymethyl cellulose to reducing carbohydrates, the reducing ability of which is determined colorimetrically by means of the ferricyanide reaction, according to Hoffman, W. S., J. Biol. Chem. 120, 51 (1937).

Cellulolytic activity may not only result in removing cellulose comprising stains but maybe advantageous to realize fabric finishing by reducing pilling, removing fibrils that make fabric surfaces rough or fuzzy, or may create stonewashed looks.

In one embodiment, the composition of the invention comprises at least one perhydrolase. Suitable “perhydrolases” are capable of catalyzing a perhydrolysis reaction that results in the production of a peracid from a carboxylic acid ester (acyl) substrate in the presence of a source of peroxygen (e.g., hydrogen peroxide). While many enzymes perform this reaction at low levels, perhydrolases exhibit a high perhydrolysis:hydrolysis ratio, often greater than 1. Suitable perhydrolases may be of plant, bacterial or fungal origin. Chemically modified or protein engineered mutants are included.

Examples of useful perhydrolases include naturally occurring Mycobacterium perhydrolase enzymes, or variants thereof. An exemplary enzyme is derived from Mycobacterium smegmatis. Such enzyme, its enzymatic properties, its structure, and variants thereof, are described in WO 2005/056782, WO 2008/063400, US 2008145353, and US 2007167344.

In one embodiment, the composition of the invention comprises at least one mannanase. “Mannanase” may be an alkaline mannanase of Family 5 or 26. It may be a wild-type from Bacillus or Humicola, particularly B. agaradhaerens, B. licheniformis, B. halodurans, B. clausii, or H. insolens. Suitable mannanases are described in WO 99/064619.

A commercially available mannanase is Mannaway® (Novozymes AIS).

In one embodiment, the composition of the invention comprises at least one peroxidase and/or oxidase. Suitable peroxidases and oxidases include those of plant, bacterial or fungal origin. Chemically modified or protein engineered mutants are included.

An oxidase according to the invention include, in particular, any laccase enzyme comprised by the enzyme classification EC 1.10.3.2, or any fragment derived therefrom exhibiting laccase activity, or a compound exhibiting a similar activity, such as a catechol oxidase (EC 1.10.3.1), an o-aminophenol oxidase (EC 1.10.3.4), or a bilirubin oxidase (EC 1.3.3.5).

Preferred laccase enzymes are enzymes of microbial origin. The enzymes may be derived from plants, bacteria or fungi (including filamentous fungi and yeasts). Suitable examples from fungi include a laccase derivable from a strain of Aspergillus, Neurospora, e.g. N. crassa, Podospora, Botrytis, Collybia, Fames, Lentinus, Pleurotus, Trametes, e.g. T. villosa and T. versicolor, Rhizoctonia, e.g. R. solani, Coprinopsis, e.g. C. cinerea, C. comatus, C. friesii, and C. plicatills, Psathyrella, e.g. P. condelleana, Panaeolus, e.g. P. papllionaceus, Myceliophthora, e.g. M. thermophlla, Schytalidium, e.g. S. thermophllum, Polyporus, e.g. P. pinsitus, Phlebia, e.g. P. radiata (WO 92/01046), or Coriolus, e.g. C. hirsutus (JP 2238885).

A laccase may be derived from Coprinopsis or Myceliophthora. In one embodiment, a laccase is derived from Coprinopsis cinerea, as disclosed in WO 97/08325; or from Myceliophthora thermophlla, as disclosed in WO 95/33836.

The laccase may be a bacterial laccase, e.g. the laccase may be a Gram positive bacterial polypeptide such as a Bacillus, Streptococcus, Streptomyces, Staphylococcus, Enterococcus, Lactobacillus, Lactococcus, Clostridium, Geobacillus, or Oceanobacillus laccase, or a Gram negative bacterial polypeptide such as an E. coli, Pseudomonas, Salmonella, Campylobacter, Helicobacter, Flavobacterium, Fusobacterium, Ilyobacter, Neisseria, or Ureaplasma laccase.

In one embodiment, laccase is selected from those as described in SEQ ID NO: 2, 4, 6, and 8 of WO 2009/127702 and variants thereof.

The term “laccase activity” is defined herein as covered by enzyme classification EC 1.10.3.2, or a similar activity, such as a catechol oxidase activity (EC 1.10.3.1), o-aminophenol oxidase activity (EC 1.10.3.4), or bilirubin oxidase activity (EC 1.3.3.5), that catalyzes the oxidation of a substrate using molecular oxygen.

“Laccase activity” is determined by oxidation of syringaldazin under aerobic conditions. The violet colour produced is measured at 530 nm. The analytical conditions are 19 μM syringaldazin, 23 mM Tris/maleate buffer, pH 7.5, 30° C., and 1 min reaction time.

Examples of other oxidases include, but are not limited to, amino acid oxidase, glucose oxidase, lactate oxidase, galactose oxidase, polyol oxidase (e.g., WO 2008/051491), and aldose oxidase. Oxidases and their corresponding substrates may be used as hydrogen peroxide generating enzyme systems, and thus a source of hydrogen peroxide. Several enzymes, such as peroxidases, haloperoxidases and perhydrolases, require a source of hydrogen peroxide. By studying EC 1.1.3._, EC 1.2.3._, EC 1.4.3._, and EC 1.5.3._ or similar classes (under the International Union of Biochemistry), other examples of such combinations of oxidases and substrates are easily recognized by one skilled in the art.

Peroxidases (EC 1.11.1.7) utilize hydrogen peroxide as substrate. Examples of useful peroxidases include peroxidases from Coprinus, e.g. from C. cinereus, and variants thereof as those described in WO 93/24618, WO 95/10602, WO 98/10060 and WO 98/15257.

Commercially available peroxidases include Guardzyme™ (Novozymes NS), PrimaGreen™ Oxy (DuPont).

“Peroxidase activity” may be measured by the ABTS method as described in Childs et al. 1975 (Biochemical J, 145, p. 93-103) and commercial kits are available from different suppliers. Other measuring methods are known to those known in the art.

A peroxidase for use in the invention also include a haloperoxidase enzyme, such as chloroperoxidase, bromoperoxidase and compounds exhibiting chloroperoxidase or bromoperoxidase activity. Haloperoxidases are classified according to their specificity for halide ions. Chloroperoxidases (E.C. 1.11.1.10) catalyze formation of hypochlorite from chloride ions. In an embodiment, the haloperoxidase is a chloroperoxidase. In one embodiment, the haloperoxidase is a vanadium haloperoxidase, i.e., a vanadate-containing haloperoxidase. In one embodiment of the present invention the vanadate-containing haloperoxidase is combined with a source of chloride ion.

Haloperoxidases have been isolated from many different fungi, in particular from the fungus group dematiaceous hyphomycetes, such as Caldariomyces, e.g., C. fumago, Alternaria, Curvularia, e.g., C. verruculosa and C. inaequalis, Drechslera, Ulocladium and Botrytis. Haloperoxidases have also been isolated from bacteria such as Pseudomonas, e.g. P. pyrrocinia, and Streptomyces, e.g. S. aureofaciens.

In one embodiment, the haloperoxidase is from Curvularia sp., in particular Curvularia verruculosa or Curvularia inaequalis, such as C. inaequalis CBS 102.42 as described in WO 95/27046; or C. verruculosa CBS 147.63 or C. verruculosa CBS 444.70 as described in WO 97/04102; or from Drechslera hartlebii as described in WO 2001/79459, Dendryphiella salina as described in WO 2001/79458, Phaeotrichoconis crotalarie as described in WO 2001/79461, or Geniculosporium sp. as described in WO 2001/79460.

In one embodiment, the composition of the invention comprises at least one lyase. “Lyase” may be a pectate lyase derived from Bacillus, particularly B. licheniformis or B. agaradhaerens, or a variant derived of any of these, e.g. as described in U.S. Pat. No. 6,124,127, WO 99/027083, WO 99/027084, WO 2002/006442, WO 2002/092741, WO 2003/095638.

Commercially available pectate lyases are Xpect™, Pectawash™ and Pectaway™ (Novozymes NS); PrimaGreen™, EcoScour (DuPont).

In one embodiment, the composition of the invention comprises at least one enzyme selected from the group of pectinases, and/or arabinases, and/or galactanases, and/or xylanases. Suitable pectinases, and/or arabinases, and/or galactanases, and/or xylanases are known to those skilled in the art.

A composition of the invention comprising components (a) and (b) and (c) is preferably liquid at 20° C. and 101.3 kPa. Such a composition may comprise component (c) in amounts in the range of 0.1 g/L to 150 g/L. In one embodiment, the composition of the invention comprises component (c) in amounts in the range of 1 g/L to 100 g/L. Preferably, the amount of component (c) in the composition of the invention is in the range of 10 g/L to 100 g/L, more preferably the amount of component (c) in the composition of the invention is in the range of 30 g/L to 90 g/L. The amount of component (c) is meant to be the total amount of enzyme comprised in the composition.

As effectiveness of inhibition of boron-containing compounds, preferably boronic acid or its derivatives, towards proteolytically active enzymes varies, the amount of component (a) in the composition preferably accommodates this purpose, and may be called “effective amount of component (a)” herein. In one embodiment, the amount of component (a) in the composition is in the range of 0.1% to 30% by weight relative to the total composition.

In a particular embodiment of the present invention, 4-FPBA is used at concentrations in the range of 0.5% to 8% by weight, or in the range of 1% to 5% by weight relative to the total composition. In another embodiment of the present invention, benzene boronic acid (BBA) is used in amounts in the range of 5% to 25% by weight relative to the total composition. In a further embodiment of the present invention, 4-(hydroxymethyl)phenylboronic acid is used in amounts in the range of 5% to 25% by weight relative to the total composition. In another embodiment of the present invention, p-tolyl-boronic acid is used in amounts in the range of 5% to 25% by weight relative to the total composition.

In one embodiment, the amount of component (b) in the composition of the invention is in the range of 10% to 65% relative to the total composition. Preferably, the amount of component (b) is in the range of 30% to 60% by weight relative to the total composition.

The composition of the invention comprises pentane-1,2-diol preferably in amounts of at least 10% by weight, more preferably in amounts of at least 20% by weight, even more preferably in amounts of at least 35% by weight, and particularly in amounts of at least 50% by weight relative to the total weight of the composition.

In one embodiment, the stability of a serine protease, preferably subtilisin, is improved during storage in the presence of component (a) and (b) when compared to the same serine protease in the presence of only component (a) and also when compared to the same serine protease in the absence of components (a) and (b).

In one embodiment, the invention provides a composition, wherein stability of a serine protease, preferably subtilisin, is improved during storage in the presence of component (a) and (b) when compared to the same serine protease in the presence of only component (a) and also when compared to the same serine protease in the absence of components (a) and (b).

To determine changes in proteolytic activity over time, the “initial proteolytic activity” of an enzyme may be measured under defined conditions at time zero (i.e. before storage) and the “proteolytic activity after storage” may be measured at a certain point in time later (i.e. after storage). The proteolytic activity after storage and after release of components (a) and/or (b) divided by the initial proteolytic activity multiplied by 100 gives the “proteolytic activity available in application” (x %). A protease is stabilized according to the invention, when its proteolytic activity available in application equals 100%. In one embodiment, proteolytic activity available in application is at least 70%, at least 75%, at least 80%, at least 85%, at least 90%, at least 91%, at least 92%, at least 93%, at least 94%, at least 95%, at least 96%, at least 97%, at least 98%, at least 99%, or at least 99.5%.

Subtracting x % from 100% gives the “loss of proteolytic activity during storage”. In one embodiment, a protease is stabilized according to the invention when essentially no loss of proteolytic activity occurs during storage, i.e. loss in proteolytic activity equals 0%. In one embodiment, essentially no loss of proteolytic activity means that the loss of proteolytic activity is less than 30%, less than 25%, less than 20%, less than 15%, less than 10%, less than 9%, less than 8%, less than 7%, less than 6%, less than 5%, less than 4%, less than 3%, less than 2%, or less than 1%.

Proteases comprised in the composition comprising components (a) and (b) may exhibit reduced proteolytic activity when compared to non-stabilized proteases. The proteolytic activity measured after adding inhibitors such as components (a) and/or (b) to component (c) divided by the initial proteolytic activity multiplied by 100 is called “residual proteolytic activity” (y %) within this invention. In one embodiment, proteases are stabilized when they do not exhibit residual proteolytic activity, i.e. y % equals 0%. In one embodiment, y % is less than 50%, less than 45%, less than 40%, less than 35%, less than 30%, less than 25%, less than 20%, less than 15%, less than 10%, less than 5%, or less than 1%.

In one embodiment, one or more enzymes other than serine proteases, preferably other than subtilisins, comprised in component (c) have improved stability. Enzymes other than serine proteases have improved stability when they retain their catalytic activity during storage in the presence of a stabilized serine protease compared to the same enzyme other than serine protease in the presence of a non-stabilized serine protease.

To determine changes in enzymatic activity of enzymes other than serine proteases over time, the “initial enzymatic activity” of an enzyme other than serine protease is measured under defined conditions at time zero (i.e. before storage) and the “enzymatic activity after storage” of an enzyme other than serine protease is measured at a certain point in time later (i.e. after storage). The enzymatic activity after storage divided by the initial enzymatic activity multiplied by 100 gives the “maintained enzymatic activity” (z %) of an enzyme other than serine protease. Preferably, such an enzyme other than serine protease is stabilized according to the invention, when its maintained enzymatic activity equals 100%. In one embodiment, maintained enzymatic activity equals at least 70%, at least 75%, at least 80%, at least 85%, at least 90%, at least 91%, at least 92%, at least 93%, at least 94%, at least 95%, at least 96%, at least 97%, at least 98%, at least 99%, or at least 99.5%.

Subtracting z % from 100% gives the “loss of enzymatic activity of an enzyme other than serine protease”. Preferably, an enzyme other than serine protease is stabilized according to the invention when essentially no loss of enzymatic activity of an enzyme other than serine protease occurs, i.e. loss in enzymatic activity of an enzyme other than serine protease equals 0%. In one embodiment, essentially no loss of enzymatic activity of an enzyme other than serine protease means that said loss of enzymatic activity is less than 30%, less than 25%, less than 20%, less than 15%, less than 10%, less than 9%, less than 8%, less than 7%, less than 6%, less than 5%, less than 4%, less than 3%, less than 2%, or less than 1%.

In one embodiment, the enzyme(s) comprised in the composition comprising components (a), (b) and (c) according to the invention, may be stabilized using additional stabilizing agents and/or protease inhibitors such as different salts like NaCl or KCl, lactic acid, formic acid or a peptide aldehydes like di-, tri- or tetrapeptide aldehydes or aldehyde analogues (either of the form B1-BO—R wherein, R is H, CH₃, CX₃, CHX₂, or CH₂X (X=halogen), BO is a single amino acid residue (in one embodiment with an optionally substituted aliphatic or aromatic side chain); and B1 consists of one or more amino acid residues (in one embodiment one, two or three), optionally comprising an N-terminal protection group, or as described in WO 09/118375 and WO 98/13459, or a protease inhibitor of the protein type such as RASI, BASI, WASI (bifunctional alpha-amylase/subtilisin inhibitors of rice, barley and wheat) or Cl₂ or SSI. In some embodiments, the enzymes comprised in the inventive composition may be stabilized by the presence of water-soluble sources of zinc (II), calcium (II) and/or magnesium (II) ions in the finished compositions that provide such ions to the enzymes, as well as other metal ions (e.g. barium (II), scandium (II), iron (II), manganese (II), aluminum (111), Tin (II), cobalt (II), copper (II), Nickel (II), and oxovanadium (IV)).

In one embodiment, the composition comprising components (a) and (b) and optionally (c) comprises a pH-adjusting compound providing a pH above 5, above 6, or above 7 when added to the liquid composition. Preferably, pH-adjusting compound provides a pH above 7.5, above 8, above 8.5, above 9, above 9.5, above 10, above 10.5, above 11, or above 11.5 when added to the liquid composition.

In one embodiment, the inventive composition comprises a pH-adjusting compound providing a pH of the liquid composition in the range of 5 to 11.5, in the range of 6 to 11.5, in the range of 7 to 11, or in the range of 8 to 11.

Suitable pH-adjusting compounds may be sodium hydroxide, potassium hydroxide or alkaline buffer salts. Suitable buffer salts may be potassium bicarbonate, potassium carbonate, tetra potassium pyrophosphate, potassium tripolyphosphate, sodium bicarbonate and sodium carbonate. Suitable might also be mixtures of pH-adjusting compounds which answer the purpose of adjusting the appropriate pH.

In one embodiment, the composition comprising components (a) and (b) and optionally (c) comprises one or more preservatives. Preservatives are normally added to liquid compositions to prevent alterations of said compositions due to attacks from microorganisms. Non-limiting examples of suitable preservatives include (quarternary) ammonium compounds, isothiazolinones, organic acids, and formaldehyde releasing agents. Non-limiting examples of suitable (quaternary) ammonium compounds include benzalkonium chlorides, polyhexamethylene biguanide (PHMB), Didecyldimethylammonium chloride(DDAC), and N-(3-aminopropyl)-N-dodecylpropane-1,3-diamine (Diamine). Non-limiting examples of suitable isothiazolinones include 1,2-benzisothiazolin-3-one (BIT), 2-methyl-2H-isothiazol-3-one (MIT), 5-chloro-2-methyl-2H-isothiazol-3-one (CIT), 2-octyl-2H-isothiazol-3-one (OIT), and 2-butyl-benzo[d]isothiazol-3-one (BBIT). Non-limiting examples of suitable organic acids include benzoic acid, sorbic acid, L-(+)-lactic acid, formic acid, and salicylic acid. Non-limiting examples of suitable formaldehyde releasing agent include N,N′-methylenebismorpholine (MBM), 2,2′,2″-(hexahydro-1,3,5-triazine-1,3,5-triyl)triethanol (HHT), (ethylenedioxy)dimethanol, .alpha.,.alpha.′,.alpha.″-trimethyl-1,3,5-triazine-1,3,5(2H,4H,6H)-triethanol (HPT), 3,3′-methylenebis[5-methyloxazolidine] (MBO), and cis-1-(3-chloroallyl)-3,5,7-triaza-1-azoniaadamantane chloride (CTAC).

Further useful preservatives include iodopropynyl butylcarbamate (IPBC), halogen releasing compounds such as dichloro-dimethyl-hydantoine (DCDMH), bromo-chloro-dimethyl-hydantoine (BCDMH), and dibromo-dimethyl-hydantoine (DBDMH); bromo-nitro compounds such as Bronopol (2-bromo-2-nitropropane-1,3-diol), 2,2-dibromo-2-cyanoacetamide (DBNPA); aldehydes such as glutaraldehyde; phenoxyethanol; Biphenyl-2-ol; and zinc or sodium pyrithione. The amount of preservative in the inventive composition depends on the actual preservative or preservative mixture used. Compositions of the invention may comprise preservatives in amounts in the range of 0,0005% to 2% relative to the total weight of the composition.

The present invention also relates to a method of preparing a composition comprising mixing in no specified order in one or more steps

component (a): at least one boron-containing compound, and

component (b): pentane-1,2-diol and optionally one or more further diols, and

optionally component (c): at least one serine protease and optionally one or more further enzymes.

In one embodiment, the composition prepared is liquid at 20° C. and 101.3 kPa. A potential residual concentration gap of the liquid composition may be filled with water. Residual gap means the restock volume to 100% of liquid composition.

Components (a), (b) and optionally (c) for preparation of the composition of the invention are those as described above. The composition of the invention may be used as stock solution for further composition preparation, such as preparation of a detergent composition.

In one aspect, the invention relates to a method of use of pentane-1,2-diol for stabilization of enzymes. The invention also relates to the use of pentane-1,2-diol for stabilization of enzymes. The present invention relates to the method of use and use of pentane-1,2-diol and optionally one or more further diols [i.e. component (b) as described above] in the presence of at least one boron-containing compound [i.e. component (a) as described above] in compositions comprising at least one serine protease and optionally one or more further enzymes [i.e. component (c) as described above] for stabilization of serine protease(s) comprised in component (c). Furthermore, the invention relates to the method of use and use of component (b) in the presence of component (a) in compositions comprising component (c) for improvement of stabilization of serine protease(s) comprised in component (c).

Furthermore, the invention involves a method of stabilization of serine protease(s), preferably subtilase(s) in compositions, wherein pentane-1,2-diol and optionally one or more further diols [i.e. component (b) as described above] is one, and at least one boron-containing compound [i.e. component (a) as described above] is another component of the composition. In one embodiment, the method is a method of improvement of protease stability of serine protease.

Improvement of protease stability in this context may mean that the protease stability is improved in the presence of pentane-1,2-diol and optionally one or more further diols [i.e. component (b) as described above] and at least one boron-containing compound [i.e. component (a) as described above], when compared to the stability of said protease in compositions comprising boron-containing compounds but lacking pentane-1,2-diol.

In one aspect of the invention, the composition comprising at least components (a) and (b) and (c) is converted to an anhydrous form e.g. by lyophilization or spray-drying e.g. in the presence of an inorganic carrier material to form aggregates. The composition comprising at least components (a) and (b) and (c) may be introduced into a granulation process such as prilling, extrusion-spheronization, high shear granulation and spray-coating as known to those skilled in the art.

In one aspect, the invention relates to microcapsules comprising at least

component (a): at least one boron-containing compound, and

component (b): pentane-1,2-diol and optionally one or more further diols, and

component (c): at least one serine proteases and optionally one or more further enzymes wherein components (a) and (b) and (c) are encapsulated within a shell (i.e. microcapsule).

Microcapsules are essentially spherical objects which consists of a core and a wall material surrounding the core. The material inside the microcapsule is referred to as the core, core composition, internal phase, or fill, whereas the membrane is sometimes called a shell, coating, or wall. According to the invention, a liquid core is surrounded by the solid wall material. For many applications the wall is formed by a polymer material.

Herein, the composition comprising at least components (a) and (b) and (c) may be part of the “core composition” of a microcapsule. In one embodiment, the composition comprising components (a) and (b) and (c) is the “core composition” of a microcapsule. In one embodiment, the core composition is liquid at 20° C. and 101.3 kPa. Components (a), (b) and (c) are those as described above.

The microcapsules of the invention have mean diameters between 0.5 μm and at most 1000 μm. Preferably, the mean diameter of the microcapsules is in the range of 1 μm to 500 μm, in the range of 10 μm to 500 μm, in the range of 50 μm to 500 μm, or in the range of 50 μm to 200 μm. The diameter of the capsule may change depending on the water activity of the surrounding chemical environment.

A multitude of shell materials is known for producing the wall of microcapsules. The shell can consist either of natural, semisynthetic or synthetic materials. Natural shell materials are, for example, gum arabic, agar agar, agarose, maltodextrins, alginic acid or its salts, e.g. sodium alginate or calcium alginate, fats and fatty acids, cetyl alcohol, collagen, chitosan, lecithins, gelatin, albumin, shellac, polysaccharides, such as starch or dextran, polypeptides, protein hydrolyzates, sucrose and waxes. Semisynthetic shell materials are inter alia chemically modified celluloses, in particular cellulose esters and cellulose ethers, e.g. cellulose acetate, ethyl cellulose, hydroxypropylcellulose, hydroxypropylmethylcellulose and carboxymethylcellulose, and also starch derivatives, in particular starch ethers and starch esters. Non-limiting examples of synthetic shell materials include polymers, such as polyacrylates, polyamides, polyesters, polyvinyl alcohols, polyvinylpyrrolidones, melamine formaldehyde, polyurethans or polyureas. Depending on the type of shell material and the production process, microcapsules are formed in each case with different properties, such as diameter, size distribution, wall thickness and physical and/or chemical properties. The aim of microencapsulation is at the one hand the isolation of the core composition from its surrounding, and on the other hand release of the core composition at the time of use (the wall must be ruptured timely). Capsule contents may be released by melting the wall, or dissolving it under particular conditions. In other systems, the wall is broken by solvent action, enzyme attack, chemical reaction, hydrolysis, or slow disintegration. Most prominently, the limiting factor for suitability in detergent formulations is a rapid release of the core composition at the time when a detergent composition is diluted in water but ensuring non-release of the core composition during storage in detergent compositions.

The ones skilled in the art are familiar with physico-chemical and chemical microencapsulation techniques such as ionotropic gelation, coacervation-phase separation, interfacial polycondensation, interfacial cross-linking, in-situ polymerization and matrix polymerization. For example, microcapsules may be formed by emulsion-based in vitro microencapsulation technology. Two main approaches are known for emulsion-based in vitro microencapsulation: oil-in-water and water-in-oil microencapsulation. Oil-in-water microencapsulation is commonly used to encapsulate non-polar active ingredients. Water-in-oil microencapsulation is employed for the encapsulation of polar (i.e. water soluble) actives such as enzymes.

Water-in-oil microencapsulation may include the following steps:
Preparation of the initial water and oil phase(s),
Forming a water-in-oil emulsion,
Membrane formation by polymerization of monomers or prepolymers at the interface of water and oil phase (interfacial polycondensation),
Optional post modification,
Optional isolation and/or formulation,
Addition to a detergent composition comprising one or more components (d).

The process can be either a batch process or a continuous or semi-continuous process. In addition to water-in-oil and oil-in-water systems water-in-water (aqueous biphasic) systems are known. Water-in-water systems can be obtained by inducing phase separation in an aqueous system containing a water-soluble polymer by for example addition of a salt, resulting in an aqueous phase containing the water-soluble polymer and another aqueous phase containing the dissolved salt.

In one embodiment, the core composition of the microcapsule additionally comprises at least one pH-adjusting compound as disclosed above providing a pH as disclosed above.

In one embodiment, the core composition of the microcapsule additionally comprises one or more preservatives as disclosed above.

In one aspect, the invention relates to detergent compositions comprising components (a) and (b) and (c) as described above, and at least one detergent component (d). “Detergent composition” or “cleaning composition” means compositions designated for cleaning soiled material.

Cleaning includes laundering and hard surface cleaning. Soiled material according to the invention includes textiles and/or hard surfaces.

The term “laundering” relates to both household laundering and industrial laundering and means the process of treating textiles with a solution containing a detergent composition of the present invention. The laundering process may be carried out by using technical devices such as a household or an industrial washing machine. Alternatively, the laundering process may be done by hand.

The term “textile” means any textile material including yarns (thread made of natural or synthetic fibers used for knitting or weaving), yarn intermediates, fibers, non-woven materials, natural materials, synthetic materials, as well as fabrics (a textile made by weaving, knitting or felting fibers) made of these materials such as garments (any article of clothing made of textile), cloths and other articles.

The term “fibers” includes natural fibers, synthetic fibers, and mixtures thereof. Examples of natural fibers are of plant (such as flax, jute and cotton) or animal origin, comprising proteins like collagen, keratin and fibroin (e.g. silk, sheeps wool, angora, mohair, cashmere). Examples for fibers of synthetic origin are polyurethane fibers such as Spandex® or Lycra®, polyester fibers, polyolefins such as elastofin, or polyamide fibers such as nylon. Fibers may be single fibers or parts of textiles such as knitwear, wovens, or nonwovens.

The term “hard surface cleaning” is defined herein as cleaning of hard surfaces wherein hard surfaces may include any hard surfaces in the household, such as floors, furnishing, walls, sanitary ceramics, glass, metallic surfaces including cutlery or dishes.

The term “dish wash” refers to all forms of washing dishes, e.g. by hand or automatic dish wash. Dish washing includes, but is not limited to, the cleaning of all forms of crockery such as plates, cups, glasses, bowls, all forms of cutlery such as spoons, knives, forks and serving utensils as well as ceramics, plastics such as melamine, metals, china, glass and acrylics.

The detergent composition of the invention comprises one or more detergent component(s). Detergent components vary in type and/or amount in a detergent composition depending on the desired application. The component(s) chosen depend on the desired cleaning application and/or physical form of a detergent composition.

The term “detergent component” is defined herein to mean the types of ingredient, which is suitable for detergent compositions, such as surfactants, building agents, polymers, bleaching systems. Any component(s) known in the art acknowledging their known characteristics are suitable detergent component(s) (d) according to the invention.

Detergent components may have more than one function in the final application of a detergent composition, therefore any detergent component mentioned in the context of a specific function herein, may also have another function in the final application of a detergent composition. The function of a specific detergent component in the final application of a detergent composition usually depends on its amount within the detergent composition, i.e. the effective amount of a detergent component.

The term “effective amount of a detergent component” herein includes (a) a detergent component's ability to effectively remove stains on an object to be cleaned [i.e. the cleaning performance of the detergent component as such] and/or (b) the contribution of a detergent component to a detergent composition's effectivity in cleaning [i.e. the cleaning performance of the detergent composition]. Preferably, a detergent composition of the invention comprises one or more detergent components in effective amounts.

Cleaning performance is evaluated under relevant cleaning conditions. The term “relevant cleaning conditions” herein refers to the conditions, particularly cleaning temperature, time, cleaning mechanics, suds concentration, type of detergent and water hardness, actually used in laundry machines, automatic dish washers or in manual cleaning processes.

The numeric ranges recited for the individual detergent components provide amounts comprised in detergent compositions. Such ranges have to be understood to be inclusive of the numbers defining the range and include each integer within the defined range.

If not described otherwise, “% by weight” or “% w/w” is meant to be related to total detergent composition. In this case “% by weight” or “% w/w” is calculated as follows: concentration of a substance as the weight of that substance divided by the total weight of the composition, multiplied by 100.

The term “about,” as used herein, refers to variation in the numerical quantity that can occur, for example, through typical measuring and liquid handling procedures used for making concentrates or use solutions in the real world; through inadvertent error in these procedures; through differences in the manufacture, source, or purity of the ingredients used to make the compositions or carry out the methods; and the like. Whether or not modified by the term “about”, the claims include equivalents to the quantities and refers to variation in the numerical quantity that can occur.

Detergent compositions of the invention may comprise inventive composition comprising at least components (a), (b) and (c) as disclosed above, wherein the amount of component (c) determines the effective amounts of component (a) and (b).

The amount of enzyme [i.e. component (c) as described above] comprised in the detergent composition is usually in the range of 0.01 g/L to 20 g/L. Particularly, the amount of component (c) in the detergent composition is in the range of 0.1 g/L to 10 g/L. The values provided preferably relate to total amount of protein in a detergent composition.

Detergent compositions of the invention preferably comprise effective amounts of boron-containing compound [i.e. component (a) as described above] in amounts in the range of 0.001% to 10% by weight relative to the total weight of the detergent composition. Effective amounts of boron-containing compound may mean amounts effective to inhibit at least one enzyme comprised in component (c).

As the amount of component (a) depends on the effectiveness of the inhibition of a proteolytic enzyme, in a particular embodiment of the present invention 4-FPBA is used in effective amounts which may be in the range of 0.005% to 0.08% by weight or 0.01% to 0.05% by weight relative to the total weight of the detergent composition. In another embodiment of the present invention benzene boronic acid is used in amounts in the range of 0.05% to1% by weight relative to the total weight of the detergent composition. In another embodiment of the present invention 4-(hydroxymethyl)phenylboronic acid is used in amounts in the range of 0.05% to 1% by weight relative to the total weight of the detergent composition. In another embodiment of the present invention p-tolyl-boronic acid is used in amounts in the range of 0.05% to 1% by weight relative to the total weight of the detergent composition. In another embodiment of the present invention boronic acid is used in amounts in the range of 0.5% to 5% by weight relative to the total weight of the detergent composition.

Detergent compositions of the invention preferably comprise effective amounts of pentane-1,2-diol [i.e. component (b) as described above], meaning amounts effective to inhibit at least one enzyme comprised in component (c). The amount of component (b) in a detergent composition of the invention preferably is in the range of 2% to 50% by weight relative to the total weight of the detergent composition. In a particular embodiment of the detergent composition, the amount of component (b) is in the range of 3% to 20% by weight, or more particularly in the range of 4% to 15% by weight, both relative to the total weight of the detergent composition.

Component (b) of the composition of the invention preferably comprises at least 10% by weight pentane-1,2-diol, more preferably at least 20% by weight pentane-1,2-diol, even more preferably at least 35% by weight pentane-1,2-diol, or in particular at least 50% by weight pentane-1,2-diol, all relative to the total weight of component (b).

The detergent composition of the invention comprising components (a) and (b) and (c) as described above, and at least one detergent component (d) as described below, may be characterized by an increased stability of component (c). Said detergent composition may be characterized by an increased stability of component (c), when compared to detergent compositions lacking components (a) and (b) in effective amounts.

Potential changes in proteolytic activity of proteases, preferably serine proteases, comprised in detergent compositions of the invention, over time (e.g. during storage) may be determined as disclosed above: The proteolytic activity after storage and after release of components (a) and/or (b) divided by the initial proteolytic activity multiplied by 100 gives the proteolytic activity available in final application (x %), wherein application in the context of detergent compositions the final application includes the ability remove protease-sensitive stains. A protease, preferably serine protease, is stabilized according to the invention, when its proteolytic activity available in final application equals 100%. In one embodiment, proteolytic activity available in application is at least 70%, at least 75%, at least 80%, at least 85%, at least 90%, at least 91%, at least 92%, at least 93%, at least 94%, at least 95%, at least 96%, at least 97%, at least 98%, at least 99%, or at least 99.5%.

Subtracting x % from 100% gives the loss of proteolytic activity during storage as disclosed above. In the context of detergent compositions, a protease, preferably serine protease, may be stabilized according to the invention, when essentially no loss of its proteolytic activity occurs during storage of the detergent composition, i.e. loss in proteolytic activity equals 0%. In one embodiment, essentially no loss of proteolytic activity means that the loss of proteolytic activity is less than 30%, less than 25%, less than 20%, less than 15%, less than 10%, less than 9%, less than 8%, less than 7%, less than 6%, less than 5%, less than 4%, less than 3%, less than 2%, or less than 1%.

Potential changes in enzymatic activity of enzymes other than serine proteases, comprised in detergent compositions of the invention, over time may be determined as disclosed above: The enzymatic activity after storage divided by the initial enzymatic activity multiplied by 100 gives the “maintained enzymatic activity” (z %) of an enzyme other than serine protease, which is available in final application of the detergent composition. Preferably, such an enzyme other than serine protease is stabilized according to the invention, when its maintained enzymatic activity equals 100%. In one embodiment, maintained enzymatic activity equals at least 70%, at least 75%, at least 80%, at least 85%, at least 90%, at least 91%, at least 92%, at least 93%, at least 94%, at least 95%, at least 96%, at least 97%, at least 98%, at least 99%, or at least 99.5%.

Subtracting z % from 100% gives the “loss of enzymatic activity of an enzyme other than serine protease”. In the context of detergent compositions, an enzyme other than serine protease may be stabilized according to the invention, when essentially no loss of enzymatic activity of an enzyme other than serine protease occurs, i.e. loss in enzymatic activity of an enzyme other than serine protease equals 0%. In one embodiment, essentially no loss of enzymatic activity of an enzyme other than serine protease means that said loss of enzymatic activity is less than 30%, less than 25%, less than 20%, less than 15%, less than 10%, less than 9%, less than 8%, less than 7%, less than 6%, less than 5%, less than 4%, less than 3%, less than 2%, or less than 1%.

The detergent composition of the invention comprises components (a), (b) and (c) as disclosed above and may comprise one or more detergent components to form detergent compositions such as exemplified below:

| | | | | laundry | | |
|---|---|---|---|---|---|---|
| | laundry | laundry | laundry | liquid unit | fabric | |
| component | solid | solid | liquid | dose | softener | ADW |
| non-ionic surfactant | x | x | x | x | x | — |
| amphoteric surfactant | x | | | | | — |
| anionic surfactant | x | x | x | x | | — |
| cationic surfactant | — | — | — | x | x | — |
| builder | x | x | x | x | — | x |
| alkaline | x | x | | | | x |
| bleaching agent | x | — | — | x | — | |
| bleach activator | | — | — | | — | |
| bleach catalyst | | — | — | | — | |
| sud suppressor | x | x | x | x | | x |
| anti-greying supplement | x | — | x | x | | |
| dye transfer inhibitor | — | x | x | — | | |
| fluorescent whitening | x | — | x | x | x | |
| agent | | | | | | |
| rheology modifier | — | — | x | x | x | x |
| preservative | — | — | x | x | x | x |
| water-soluble alcohol | x | — | x | x | x | x |
| hydrotropes | | | x | x | | |
| corrosion inhibitor | x | x | x | x | | x |

The detergent composition of the invention may comprise a total amount of non-ionic surfactants in the range of 0% to about 40% by weight, in the range of about 0.2% to about 30% by weight, in the range of about 0.5% to about 25% by weight, in the range of about 1% to about 15% by weight, in the range of about 3% to about 5% by weight, or in the range of about 8% to about 12% by weight, all relative to the total weight of the detergent composition.

The detergent composition of the invention may comprise a total amount of amphoteric surfactants in the range of about 0.05% to about 10% by weight, in the range of about 0.1 to about 8% by weight, or in the range of about 0.5% to about 5% by weight, all relative to the total weight of the detergent composition.

The detergent composition of the invention may comprise a total amount of anionic surfactants in the range of about 1% to about 50% by weight, in the range of about 3% to about 40% by weight, in the range of about 5% to about 30% by weight, or in the range of about 10% to about 25% by weight, all relative to the total weight of the detergent composition.

The detergent composition of the invention may comprise a total amount of cationic surfactants in the range of about 0.05% to about 15% by weight, in the range of about 0.1 to about 10% by weight, or in the range of about 0.5% to about 8% by weight, all relative to the total weight of the detergent composition.

Solid detergent compositions may comprise a total amount of builders in the range of 0% to about 60% by weight, in the range of about 1% to about 50% by weight, or up to about 20% by weight, all relative to the total weight of the detergent composition.

Liquid detergent compositions may comprise a total amount of builders in the range of 0% to about 20% by weight, in the range of about 1% to about 15% by weight, in the range of about 5% to about 10% by weight, or in the range of about 5% to about 8% by weight, all relative to the total weight of the detergent composition.

The detergent composition of the invention may comprise total amounts of pH-adjusting compounds, which may be called alkalis herein, in the range of 0% to 25% by weight, in the range of 2% to 20% by weight, or in the range of 5% to 15% by weight, all relative to the total weight of the detergent composition.

The detergent composition of the invention may comprise total amounts of suds suppressors in the range of 0% to 10% by weight, in the range of 0.1% to 5% by weight, or in the range of 1% to 3% by weight, all relative to the total weight of the detergent composition.

The detergent composition of the invention may comprise a total amount of anti-redeposition agents, which may be called anti-greying agents herein, in the range of 0% to 10% by weight, or in the range of 0.1% to 1% by weight, both relative to the total weight of the detergent composition.

The detergent composition of the invention may comprise a total amount of dye-transfer inhibition agents in the range of 0% to 2% by weight, or 0.05% to 0.5% by weight, both relative to the total weight of the detergent composition.

The detergent composition of the invention, preferably solid detergent compositions, may comprise a total amount of chlorine beaches in the range of about 0.01% to about 10% by weight, or in the range of about 0.3% to about 10% by weight, all relative to the total weight of the detergent composition.

The detergent composition of the invention, preferably solid detergent compositions, may comprise a total amount of peroxide in the range of 0.5% to 30% by weight, in the range of 1% to 20% by weight, or in the range of 2% to 15% by weight, all relative to the total weight of the detergent composition. In one embodiment peroxide comprised in a detergent composition is below 5% by weight relative to the total weight of the detergent composition.

The detergent composition of the invention, preferably solid detergent compositions, may comprise a total amount of photobleaches in the range of 0.01% to 10% by weight, in the range of 0.01% to 5% by weight, or in the range of 0.01% to 2% by weight, all relative to the total weight of the detergent composition.

The detergent composition of the invention, preferably solid detergent compositions, may comprise a total amount of bleach activators in the range of 0.5% to 10% by weight, in the range of 0.5% to 8% by weight, or in the range of 1% to 8% by weight, all relative to the total weight of the detergent composition.

The detergent composition of the invention, preferably solid detergent compositions, may comprise a total amount of bleach catalyst in the range of 0.005% to 2% by weight, in the range of 0.01% to 2% by weight, or in the range of 0.01% to 1% by weight, all relative to the total weight of the detergent composition.

The detergent composition of the invention may comprise a total amount of fluorescent whitening in the range of 0.001% to 5% by weight, in the range of 0.01% to 2% by weight, or in the range of 0.05% to 1% by weight, relative to the total weight of the detergent composition. The detergent composition of the invention, preferably liquid detergent compositions, may comprise a total amount of preservatives in the range of 0,0005% to 2% relative to the total weight of the composition. The amount of preservative in the inventive composition depends on the actual preservative or preservative mixture used.

The detergent composition of the invention, preferably liquid detergent compositions, may comprise a total amount of thickeners in amounts in the range of about 0.005% to about 5% by weight, in the range of about 0.01% to about 5% by weight, in the range of about 0.01% to about 1% by weight in the range of about 0.05% to about 0.8% by weight, in the range of about 0.1% to about 0.6% by weight, or in the range of about 0.3% to about 0.5% by weight, all relative to the total weight of the detergent composition.

The detergent composition of the invention may comprise hydrotropes in amounts in the range of 0% to 10%, relative to the total amount of the detergent composition.

The detergent composition of the invention may comprise a total amount of corrosion inhibitors in the range of 0% to 15% by weight, or 0.1% to 10% by weight, or 0.1% to 5%, or 0.1% to 1.5% by weight, all relative to the total weight of the detergent composition.

Detergent compositions designated for automated dish washing (ADW) may be free from surfactants. Free from surfactants shall mean, in the context of the present invention, that the total contents of surfactants is 0.1% by weight or less, relative to the total weight of the detergent composition. Such compositions may also be free from organic polymers such as polyacrylates, polyethylene imines, and polyvinylpyrrolidone (molecular weight (M_{w}) of 1,000 g or more). Free from organic polymers shall mean, in the context of the present invention, that the total contents of organic polymers is 0.1% by weight or less, relative to the total weight of the detergent composition. ADW detergent compositions may not contain major amounts of alkali metal of mono- and dicarboxylic acids such as acetic acid, propionic acid, maleic acid, acrylic acid, adipic acid, succinic acid, and the like. Major amounts in this context refer to amounts over 0.5% by weight relative to the total weight of the detergent composition.

Detergent compositions of the invention may comprise one or more surfactant(s). “Surfactant” (synonymously used herein with “surface active agent”) means an organic chemical that, when added to a liquid, changes the properties of that liquid at an interface. According to its ionic charge, a surfactant is called non-ionic, anionic, cationic, or amphoteric.

Non-limiting examples of surfactants are disclosed McCutcheon's 2016 Detergents and Emulsifiers, and McCutcheon's 2016 Functional Materials, both North American and International Edition, MC Publishing Co, 2016 edition. Further useful examples are disclosed in earlier editions of the same publications which are known to those skilled in the art.

Non-ionic surfactant means a surfactant that contains neither positively nor negatively charged (i.e. ionic) functional groups. In contrast to anionic and cationic surfactants, non-ionic surfactants do not ionize in solution.

Examples provided below for surfactants of any kind are to be understood to be non-limiting. Non-ionic surfactants may be compounds of the general formulae (Ia) and (Ib):

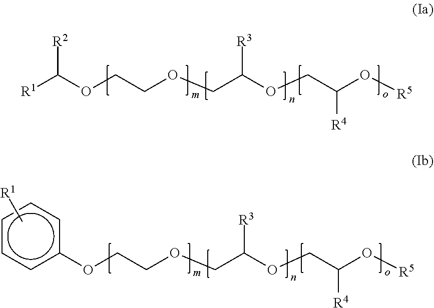

The variables of the general formulae (Ia) and (Ib) are defined as follows:

R¹ is selected from C₁-C₂₃ alkyl and C₂-C₂₃ alkenyl, wherein alkyl and/or alkenyl are linear or branched; examples are n-C₇H₁₅, n-C₉H₁₉, n-C₁₁H₂₃, n-C₁₃H₂₇, n-C₁₅H₃₁, n-C₁₇H₃₅, i-C₉H₁₉, i-C₁₂H₂₅.

R² is selected from H, C₁-C₂₀ alkyl and C₂-C₂₀ alkenyl, wherein alkyl and/or alkenyl are linear or branched.

R³ and R⁴, each independently selected from C₁-C₁₆ alkyl, wherein alkyl is linear or branched; examples are methyl, ethyl, n-propyl, isopropyl, n-butyl, isobutyl, sec-butyl, tert-butyl, n-pentyl, isopentyl, sec-pentyl, neopentyl, 1,2-dimethylpropyl, isoamyl, n-hexyl, isohexyl, sec-hexyl, n-heptyl, n-octyl, 2-ethylhexyl, n-nonyl, n-decyl, isodecyl.

R⁵ is selected from H and C₁-C₁₈ alkyl, wherein alkyl is linear or branched.

The integers of the general formulae (Ia) and (Ib) are defined as follows:

m is in the range of zero to 200, preferably 1-80, more preferably 3-20; n and o, each independently in the range of zero to 100; n preferably is in the range of 1 to 10, more preferably 1 to 6; o preferably is in the range of 1 to 50, more preferably 4 to 25. The sum of m, n and o is at least one, preferably the sum of m, n and o is in the range of 5 to 100, more preferably in the range of from 9 to 50.

The non-ionic surfactants of the general formula (I) may be of any structure, is it block or random structure, and is not limited to the displayed sequence of formula (I).

Non-ionic surfactants may further be compounds of the general formula (II), which might be called alkyl-polyglycosides (APG):

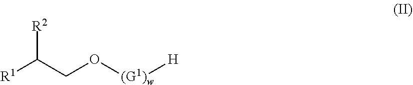

The variables of the general formula (II) are defined as follows:

R¹ is selected from C₁-C₁₇ alkyl and C₂-C₁₇ alkenyl, wherein alkyl and/or alkenyl are linear or branched; examples are n-C₇H₁₅, n-C₉H₁₉, n-C₁₁H₂₃, n-C₁₃H₂₇, n-C₁₅H₃₁, n-C₁₇H₃₅, i-C₉H₁₉, i-C₁₂H₂₅.

R² is selected from H, C₁-C₁₇ alkyl and C₂-C₁₇ alkenyl, wherein alkyl and/or alkenyl are linear or branched.

G¹ is selected from monosaccharides with 4 to 6 carbon atoms, such as glucose and xylose.

The integer w of the general formula (II) is in the range of from 1.1 to 4, w being an average number.

Non-ionic surfactants may further be compounds of general formula (III):

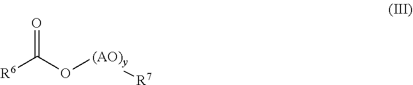

The variables of the general formula (III) are defined as follows:

AO is selected from ethylene oxide (EO), propylene oxide (PO), butylene oxide (BO), and mixtures thereof.

R⁶ is selected from C₅-C₁₇ alkyl and C₅-C₁₇ alkenyl, wherein alkyl and/or alkenyl are linear or branched.

R⁷ is selected from H, C₁-C₁₈-alkyl, wherein alkyl is linear or branched.

The integer y of the general formula (III) is a number in the range of 1 to 70, preferably 7 to 15.

Non-ionic surfactants may further be selected from sorbitan esters and/or ethoxylated or propoxylated sorbitan esters. Non-limiting examples are products sold under the trade names SPAN and TWEEN.

Non-ionic surfactants may further be selected from alkoxylated mono- or di-alkylamines, fatty acid monoethanolamides (FAMA), fatty acid diethanolamides (FADA), ethoxylated fatty acid monoethanolamides (EFAM), propoxylated fatty acid monoethanolamides (PFAM), polyhydroxy alkyl fatty acid amides, or N-acyl N-alkyl derivatives of glucosamine (glucamides, GA, or fatty acid glucamide, FAGA), and combinations thereof.

Mixtures of two or more different non-ionic surfactants may also be present in detergent compositions according to the present invention.

Amphoteric surfactants are those, depending on pH, which can be either cationic, zwitterionic or anionic.

Surfactants may be compounds comprising amphoteric structures of general formula (IV), which might be called modified amino acids (proteinogenic as well as non-proteinogenic):

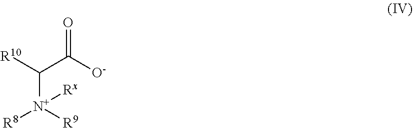

The variables in general formula (IV) are defined as follows:

R⁸ is selected from H, C₁-C₄ alkyl, C₂-C₄ alkenyl, wherein alkyl and/or are linear or branched.

R⁹ is selected from C₁-C₂₂-alkyl, C₂-C₂₂-alkenyl, C₁₀-C₂₂ alkylcarbonyl, and C₁₀-C₂₂ alkenylcarbonyl.

R¹⁰ is selected from H, methyl, —(CH₂)₃NHC(NH)NH₂, —CH₂C(O)NH₂, —CH₂C(O)OH, —(CH₂)₂C(O)NH₂, —(CH₂)₂C(O)OH, (imidazole-4-yl)-methyl, —CH(CH₃)C₂H₅, —CH₂CH(CH₃)₂, —(CH₂)₄NH₂, benzyl, hydroxymethyl, —CH(OH)CH₃, (indole-3-yl)-methyl, (4-hydroxy-phenyl)-methyl, isopropyl, —(CH₂)₂SCH₃, and —CH₂SH.

R^{x} is selected from H and C₁-C₄-alkyl.

Surfactants may further be compounds comprising amphoteric structures of general formulae (Va), (Vb), or (Vc), which might be called betaines and/or sulfobetaines:

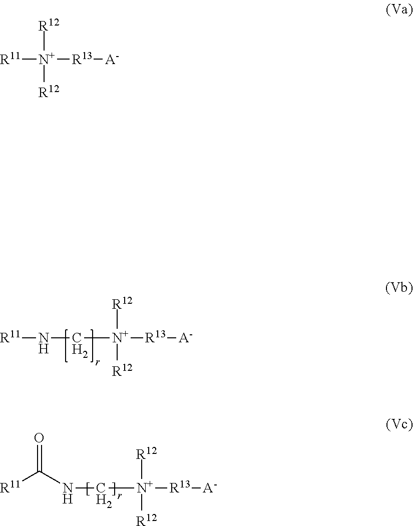

The variables in general formulae (Va), (Vb) and (Vc) are defined as follows:

R¹¹ is selected from linear or branched C₇-C₂₂ alkyl and linear or branched C₇-C₂₂ alkenyl.

R¹² are each independently selected from linear C₁-C₄ alkyl.

R¹³ is selected from C₁-C₅ alkyl and hydroxy C₁-C₅ alkyl; for example 2-hydroxypropyl.

A⁻ is selected from carboxylate and sulfonate.

The integer r in general formulae (Va), (Vb), and (Vc) is in the range of 2 to 6.

Surfactants may further be compounds comprising amphoteric structures of general formula (VI), which might be called alkyl-amphocarboxylates:

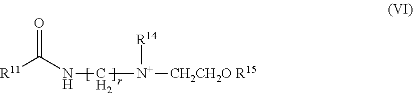

The variables in general formula (VI) are defined as follows:

R¹¹ is selected from C₇-C₂₂ alkyl and C₇-C₂₂ alkenyl, wherein alkyl and/or alkenyl are linear or branched, preferably linear.

R¹⁴ is selected from —CH₂C(O)O⁻M⁺, —CH₂CH₂C(O)O⁻M⁺ and —CH₂CH(OH)CH₂SO₃⁻M⁺.

R¹⁵ is selected from H and —CH₂C(O)O⁻

The integer r in general formula (VI) is in the range of 2 to 6.

Non-limiting examples of further suitable alkyl-amphocarboxylates include sodium cocoamphoacetate, sodium lauroamphoacetate, sodium capryloamphoacetate, disodium cocoamphodiacetate, disodium lauroamphodiacetate, disodium caprylamphodiacetate, disodium capryloamphodiacetate, disodium cocoamphodipropionate, disodium lauroamphodipropionate, disodium caprylamphodipropionate, and disodium capryloamphodipropionate.

Surfactants may further be compounds comprising amphoteric structures of general formula (VII), which might be called amine oxides (AO):

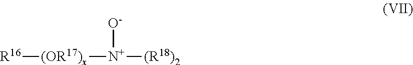

The variables in general formula (VII) are defined as follows:

R¹⁶ is selected from C₈-C₁₈ linear or branched alkyl, hydroxy C₈-C₁₈ alkyl, acylamidopropoyl and C₈-C₁₈ alkyl phenyl group; wherein alkyl and/or alkenyl are linear or branched.

R¹⁷ is selected from C₂-C₃ alkylene, hydroxy C₂-C₃ alkylene, and mixtures thereof.

R¹⁸: each residue can be independently selected from C₁-C₃ alkyl and hydroxy C₁-C_{3;} R¹⁵ groups can be attached to each other, e.g., through an oxygen or nitrogen atom, to form a ring structure.

The integer x in general formula (VII) is in the range of 0 to 5, preferably from 0 to 3, most preferably 0.

Non-limiting examples of further suitable amine oxides include C₁₀-C₁₈ alkyl dimethyl amine oxides and C₈-C₁₈ alkoxy ethyl dihydroxyethyl amine oxides. Examples of such materials include dimethyloctyl amine oxide, diethyldecyl amine oxide, bis-(2-hydroxyethyl)dodecyl amine oxide, dimethyldodecylamine oxide, dipropyltetradecyl amine oxide, methylethylhexadecyl amine oxide, dodecylamidopropyl dimethyl amine oxide, cetyl dimethyl amine oxide, stearyl dimethyl amine oxide, tallow dimethyl amine oxide and dimethyl-2-hydroxyoctadecyl amine oxide.

A further example of a suitable amine oxide is cocamidylpropyl dimethylaminoxide, sometimes also called cocamidopropylamine oxide.

Mixtures of two or more different amphoteric surfactants may be present in detergent compositions according to the present invention.

Anionic surfactant means a surfactant with a negatively charged ionic group. Anionic surfactants include, but are not limited to, surface-active compounds that contain a hydrophobic group and at least one water-solubilizing anionic group, usually selected from sulfates, sulfonate, and carboxylates to form a water-soluble compound.

Anionic surfactants may be compounds of general formula (VIII), which might be called (fatty) alcohol/alkyl (ethoxy/ether) sulfates [(F)A(E)S] when A⁻ is SO₃⁻, (fatty) alcohol/alkyl (ethoxy/ether) carboxylat [(F)A(E)C] when A⁻ is —RCOO⁻:

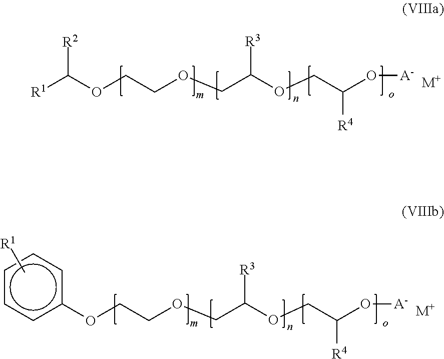

The variables in general formulae (VIIIa and VIIIb) are defined as follows:

R¹ is selected from C₁-C₂₃-alkyl (such as 1-, 2-, 3-, 4-C₁-C₂₃-alkyl) and C₂-C₂₃-alkenyl, wherein alkyl and/or alkenyl are linear or branched, and wherein 2-, 3-, or 4-alkyl; examples are n-C₇H₁₅, n-C₉H₁₉, n-C₁₁H₂₃, n-C₁₃H₂₇, n-C₁₅H₃₁, n-C₁₇H₃₅, i-C₉H₁₉, i-C₁₂H₂₅.

R² is selected from H, C₁-C₂₀-alkyl and C₂-C₂₀-alkenyl, wherein alkyl and/or alkenyl are linear or branched.

R³ and R⁴, each independently selected from C₁-C₁₆-alkyl, wherein alkyl is linear or branched; examples are methyl, ethyl, n-propyl, isopropyl, n-butyl, isobutyl, sec-butyl, tert-butyl, n-pentyl, isopentyl, sec-pentyl, neopentyl, 1,2-dimethylpropyl, isoamyl, n-hexyl, isohexyl, sec-hexyl, n-heptyl, n-octyl, 2-ethylhexyl, n-nonyl, n-decyl, isodecyl.

A⁻ is selected from —RCOO⁻, —SO₃⁻ and RSO₃⁻, wherein R is selected from linear or branched C₁-C₈-alkyl, and C₁-C₄ hydroxyalkyl, wherein alkyl is.

M⁺ is selected from H and salt forming cations. Salt forming cations may be monovalent or multivalent; hence M⁺ equals 1/v M^{v+}. Examples include but are not limited to sodium, potassium, magnesium, calcium, ammonium, and the ammonium salt of mono-, di, and triethanolamine. The integers of the general formulae (VIIIa) and (VIIIb) are defined as follows:

m is in the range of zero to 200, preferably 1-80, more preferably 3-20; n and o, each independently in the range of zero to 100; n preferably is in the range of 1 to 10, more preferably 1 to 6; o preferably is in the range of 1 to 50, more preferably 4 to 25. The sum of m, n and o is at least one, preferably the sum of m, n and o is in the range of 5 to 100, more preferably in the range of from 9 to 50.

Anionic surfactants of the general formula (VIII) may be of any structure, block copolymers or random copolymers.

Further suitable anionic surfactants include salts (M⁺) of C₁₂-C₁₈ sulfo fatty acid alkyl esters (such as C₁₂-C₁₈ sulfo fatty acid methyl esters), C₁₀-C₁₈-alkylarylsulfonic acids (such as n-C₁₀-C₁₈-alkylbenzene sulfonic acids) and C₁₀-C₁₈ alkyl alkoxy carboxylates.

M⁺ in all cases is selected from salt forming cations. Salt forming cations may be monovalent or multivalent; hence M⁺ equals 1/v M^{v+}. Examples include but are not limited to sodium, potassium, magnesium, calcium, ammonium, and the ammonium salt of mono-, di, and triethanolamine.

Non-limiting examples of further suitable anionic surfactants include branched alkylbenzenesulfonates (BABS), phenylalkanesulfonates, alpha-olefinsulfonates (AOS), olefin sulfonates, alkene sulfonates, alkane-2,3-diylbis(sulfates), hydroxyalkanesulfonates and disulfonates, secondary alkanesulfonates (SAS), paraffin sulfonates (PS), sulfonated fatty acid glycerol esters, alkyl- or alkenylsuccinic acid, fatty acid derivatives of amino acids, diesters and monoesters of sulfo-succinic acid.

Anionic surfactants may be compounds of general formula (IX), which might be called N-acyl amino acid surfactants:

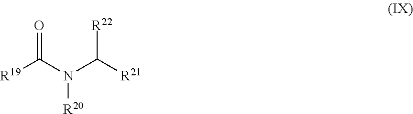

The variables in general formula (IX) are defined as follows:

R¹⁹ is selected from linear or branched C₆-C₂₂-alkyl and linear or branched C₆-C₂₂-alkenyl such as oleyl.

R²⁰ is selected from H and C₁-C₄-alkyl.

R²¹ is selected from H, methyl, —(CH₂)₃NHC(NH)NH₂, —CH₂C(O)NH₂, —CH₂C(O)OH, —(CH₂)₂C(O)NH₂, —(CH₂)₂C(O)OH, (imidazole-4-yl)-methyl, —CH(CH₃)C₂H₅, —CH₂CH(CH₃)₂, —(CH₂)₄NH₂, benzyl, hydroxymethyl, —CH(OH)CH₃, (indole-3-yl)-methyl, (4-hydroxy-phenyl)-methyl, isopropyl, —(CH₂)₂SCH₃, and —CH₂SH.

R²² is selected from —COOX and —CH₂SO₃X, wherein X is selected from Li⁺, Na⁺ and K⁺.

Non-limiting examples of suitable N-acyl amino acid surfactants are the mono- and di-carboxylate salts (e.g., sodium, potassium, ammonium and ammonium salt of mono-, di, and triethanolamine) of N-acylated glutamic acid, for example, sodium cocoyl glutamate, sodium lauroyl glutamate, sodium myristoyl glutamate, sodium palmitoyl glutamate, sodium stearoyl glutamate, disodium cocoyl glutamate, disodium stearoyl glutamate, potassium cocoyl glutamate, potassium lauroyl glutamate, and potassium myristoyl glutamate; the carboxylate salts (e.g., sodium, potassium, ammonium and ammonium salt of mono-, di, and triethanolamine) of N-acylated alanine, for example, sodium cocoyl alaninate, and triethanolamine lauroyl alaninate; the carboxylate salts (e.g., sodium, potassium, ammonium and ammonium salt of mono-, di, and triethanolamine) of N-acylated glycine, for example, sodium cocoyl glycinate, and potassium cocoyl glycinate; the carboxylate salts (e.g., sodium, potassium, ammonium and ammonium salt of mono-, di, and triethanolamine) of N-acylated sarcosine, for example, sodium lauroyl sarcosinate, sodium cocoyl sarcosinate, sodium myristoyl sarcosinate, sodium oleoyl sarcosinate, and ammonium lauroyl sarcosinate.

Anionic surfactants may further be selected from the group of soaps. Suitable are salts (M⁺) of saturated and unsaturated C₁₂-C₁₈ fatty acids, such as lauric acid, myristic acid, palmitic acid, stearic acid, behenic acid, oleic acid, (hydrated) erucic acid. M⁺ is selected from salt forming cations. Salt forming cations may be monovalent or multivalent; hence M⁺ equals 1/v M^{v+}. Examples include but are not limited to sodium, potassium, magnesium, calcium, ammonium, and the ammonium salt of mono-, di, and triethanolamine.

Further non-limiting examples of suitable soaps include soap mixtures derived from natural fatty acids such as tallow, coconut oil, palm kernel oil, laurel oil, olive oil, or canola oil. Such soap mixtures comprise soaps of lauric acid and/or myristic acid and/or palmitic acid and/or stearic acid and/or oleic acid and/or linoleic acid in different amounts, depending on the natural fatty acids from which the soaps are derived.

Further non-limiting examples of suitable anionic surfactants include salts (M+) of sulfates, sulfonates or carboxylates derived from natural fatty acids such as tallow, coconut oil, palm kernel oil, laurel oil, olive oil, or canola oil. Such anionic surfactants comprise sulfates, sulfonates or carboxylates of lauric acid and/or myristic acid and/or palmitic acid and/or stearic acid and/or oleic acid and/or linoleic acid in different amounts, depending on the natural fatty acids from which the soaps are derived.

Mixtures of two or more different anionic surfactants may also be present in detergent compositions according to the present invention.

Mixtures of non-ionic and/or amphoteric and/or anionic surfactants may also be present in detergent compositions according to the present invention.

Cationic surfactant means a surfactant with a positively charged ionic group.

Typically, these cationic moieties are nitrogen containing groups such as quaternary ammonium or protonated amino groups. The cationic protonated amines can be primary, secondary, or tertiary amines.

Cationic surfactants may be compounds of the general formula (X) which might be called quaternary ammonium compounds (quats):

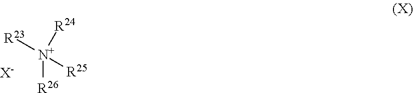

The variables in general formula (X) are defined as follows:

R²³ is selected from H, C₁-C₄ alkyl (such as methyl) and C₂-C₄ alkenyl, wherein alkyl and/or alkenyl is linear or branched.

R²⁴ is selected from C₁-C₄ alkyl (such as methyl), C₂-C₄ alkenyl and C₁-C₄ hydroxyalkyl (such as hydroxyethyl), wherein alkyl and/or alkenyl is linear or branched.

R²⁵ is selected from C₁-C₂₂ alkyl (such as methyl, C₁₈ alkyl), C₂-C₄ alkenyl, C₁₂-C₂₂ alkylcarbonyloxymethyl and C₁₂-C₂₂ alkylcarbonyloxyethyl (such as C₁₆-C₁₈ alkylcarbonyloxyethyl), wherein alkyl and/or alkenyl is linear or branched.

R²⁶ is selected from C₁₂-C₁₈ alkyl, C₂-C₄ alkenyl, C₁₂-C₂₂ alkylcarbonyloxymethyl, C₁₂-C₂₂ alkylcarbonyloxyethyl and 3-(C₁₂-C₂₂ alkylcarbonyloxy)-2(C₁₂-C₂₂ alkylcarbonyloxy)-propyl.

X⁻ is selected from halogenid, such as Cl⁻ or Br.

Non-limiting examples of further cationic surfactants include, amines such as primary, secondary and tertiary monoamines with C₁₈ alkyl or alkenyl chains, ethoxylated alkylamines, alkoxylates of ethylenediamine, imidazoles (such as 1-(2-hydroxyethyl)-2-imidazoline, 2-alkyl-1-(2-hydroxyethyl)-2-imidazoline, and the like), quaternary ammonium salts like alkylquaternary ammonium chloride surfactants such as n-alkyl(C₁₂-C₁₈)dimethylbenzyl ammonium chloride, n-tetradecyldimethylbenzylammonium chloride monohydrate, and a naphthylene-substituted quaternary ammonium chloride such as dimethyl-1-naphthylmethylammonium chloride.

Particularly suitable cationic surfactants that may be:
N,N-dimethyl-N-(hydroxy-C₇-C₂₅-alkyl)ammonium salts;
mono- and di(C₇-C₂₅-alkyl)dimethylammonium compounds quaternized with alkylating agents;
ester quats, in particular quaternary esterified mono-, di- and trialkanolamines which are esterified with C₈-C₂₂-carboxylic acids;
imidazoline quats, in particular 1-alkylimidazolinium salts of formulae XI or XII

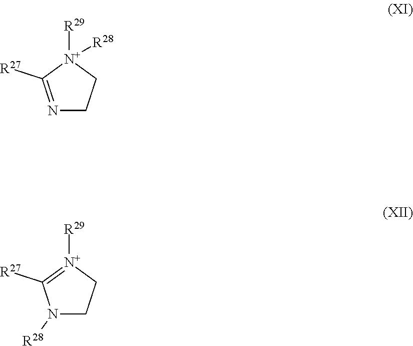

The variables in formulae (XI) and (XII) are defined as follows:

R²⁷ is selected from C₁-C₂₅-alkyl and C₂-C₂₅-alkenyl;

R²⁸ is selected from C₁-C₄-alkyl and hydroxy-C₁-C₄-alkyl;

R²⁹ is selected from C₁-C₄-alkyl, hydroxy-C₁-C₄-alkyl and a R*—(CO)—R³⁰—(CH₂)ⱼ— radical, wherein

R* is selected from C₁-C₂₁-alkyl and C₂-C₂₁-alkenyl; R³⁰ is selected from-O— and —NH—; j is 2 or 3.

Detergent compositions of the invention may comprise one or more compounds selected from complexing agents (chelating agents, sequestrating agents), precipitating agents, and ion exchange compounds, which may form water-soluble complexes with Ca and Mg. Such compounds may be called “builders” or “building agents” herein, without meaning to limit such compounds to this function in the final application of a detergent composition.

Builders used in detergent compositions of the invention may be selected from phosphate based builders. The term “phosphate(s)” includes, but is not limited to sodium metaphosphate, sodium orthophosphate, sodium hydrogenphosphate, sodium pyrophosphate, trisodium phosphate, pentasodium tripolyphosphate, hexasodium metaphosphate, and polyphosphates such as sodium tripolyphosphate.

Preferably, detergent compositions of the current invention are free from phosphate, meaning essentially free from phosphate based builders. Herein, “essentially free from phosphate” is to be understood, as meaning that the content of phosphate and polyphosphate is in sum in the range of 10 ppm to 1% by weight, determined by gravimetry and referring to the respective inventive detergent composition.

Non-phosphate based builders according to the invention include sodium gluconate, citrate(s), silicate(s), carbonate(s), phosphonate(s), amino carboxylate(s), polycarboxylate(s), polysulfonate(s), and polyphosphonate(s).

Detergent compositions of the invention may comprise one or more citrates. The term “citrate(s)” includes the mono- and the dialkali metal salts and in particular the mono- and preferably the trisodium salt of citric acid, ammonium or substituted ammonium salts of citric acid as well as citric acid as such. Citrate can be used as the anhydrous compound or as the hydrate, for example as sodium citrate dihydrate.

Detergent compositions of the invention may comprise one or more silicates. “Silicate(s)” in the context of the present invention include in particular sodium disilicate and sodium metasilicate, aluminosilicates such as sodium aluminosilicates like zeolith A (i.e. Na₁₂(AlO₂)₁₂(SiO₂)₁₂*27H₂O), and sheet silicates, in particular those of the formula alpha-Na₂Si₂O₅, beta-Na₂Si₂O₅, and delta-Na₂Si₂O₅.

Detergent compositions of the invention may comprise one or more carbonates. The term “carbonate(s)” includes alkali metal carbonates and alkali metal hydrogen carbonates, preferred are the sodium salts. Particularly suitable is sodium carbonate (Na₂CO₃).

Detergent compositions of the invention may comprise one or more phosphonates. “Phosphonates” include, but are not limited to 2-phosphinobutane-1,2,4-tricarboxylic acid (PBTC); ethylenediaminetetra(methylenephosphonic acid) (EDTMPA; 1-hydroxyethane-1,1-diphosphonic acid (HEDP), CH₂C(OH)[PO(OH)₂]₂; aminotris(methylenephosphonic acid) (ATMP), N[CH₂PO(OH)₂]₃; aminotris(methylenephosphonate), sodium salt (ATMP), N[CH₂PO(ONa)₂]₃; 2-hydroxyethyliminobis(methylenephosphonic acid), HOCH₂CH₂N[CH₂PO(OH)₂]₂; diethylenetriaminepenta(methylenephosphonic acid) (DTPMP), (HO)₂POCH₂N[CH₂CH₂N[CH₂PO(OH)₂]₂]₂; diethylenetriaminepenta(methylenephosphonate), sodium salt, C₉H₍₂₈₋ₓ₎N₃NaₓO₁₅P₅ (x=7); hexamethylenediamine(tetramethylenephosphonate), potassium salt, C₁₀H₍₂₈₋ₓ₎N₂KₓO₁₂P₄ (x=6); and bis(hexamethylene)triamine(pentamethylenephosphonic acid), (HO₂)POCH₂N[(CH₂)₂N[CH₂PO(OH)₂]₂]₂. Salts thereof may be suitable, too. Detergent compositions of the invention may comprise one or more aminocarboxylates. Non-limiting examples of suitable “amino carboxylates” include, but are not limited to: diethanol glycine (DEG), dimethylglycine (DMG), nitrilitriacetic acid (NTA), N-hydroxyethylaminodiacetic acid, ethylenediaminetetraacetic acid (EDTA), N-(2hydroxyethyl)iminodiacetic acid (HEIDA), hydroxyethylenediaminetriacetic acid, N-hydroxyethyl-ethylenediaminetriacetic acid (HEDTA), hydroxyethylenediaminetetraacetic acid, diethylenetriaminepentaacetic acid (DTPA), and methylglycinediacetic acid (MGDA), glutamic acid-diacetic acid (GLDA), iminodisuccinic acid (IDS), hydroxyiminodisuccinic acid, ethylenediaminedisuccinic acid (EDDS), aspartic acid-diacetic acid, and alkali metal salts or ammonium salts thereof. Further suitable are aspartic acid-N-monoacetic acid (ASMA), aspartic acid-N,N-diacetic acid (ASDA), aspartic acid-N-monopropionic acid (ASMP), N-(2-sulfomethyl) aspartic acid (SMAS), N-(2-sulfoethyl) aspartic acid (SEAS), N-(2-sulfomethyl) glutamic acid (SMGL), N-(2-sulfoethyl) glutamic acid (SEGL), N-methyliminodiacetic acid (MIDA), alpha-alanine-N,N-diacetic acid (alpha-ALDA), serine-N,N-diacetic acid (SEDA), isoserine-N,N-diacetic acid (ISDA), phenylalanine-N,N-diacetic acid (PHDA), anthranilic acid-N,N-diacetic acid (ANDA), sulfanilic acid-N,N-diacetic acid (SLDA), taurine-N,N-diacetic acid (TUDA) and sulfomethyl-N,N-diacetic acid (SMDA) and alkali metal salts or ammonium salts thereof. The term “ammonium salts” as used in in this context refers to salts with at least one cation that bears a nitrogen atom that is permanently or temporarily quaternized. Examples of cations that bear at least one nitrogen atom that is permanently quaternized include tetramethylammonium, tetraethylammonium, dimethyldiethyl ammonium, and n-C₁₀-C₂₀-alkyl trimethyl ammonium. Examples of cations that bear at least one nitrogen atom that is temporarily quaternized include protonated amines and ammonia, such as monomethyl ammonium, dimethyl ammonium, trimethyl ammonium, monoethyl ammonium, diethyl ammonium, triethyl ammonium, n-C₁₀-C₂₀-alkyl dimethyl ammonium 2-hydroxyethylammonium, bis(2-hydroxyethyl) ammonium, tris(2-hydroxyethyl)ammonium, N-methyl 2-hydroxyethyl ammonium, N,N-dimethyl-2-hydroxyethylammonium, and especially NH₄⁺.

In one embodiment, detergent compositions of the invention comprise more than one builder.

Preferably, inventive detergent compositions contain less than 0.2% by weight of nitrilotriacetic acid (NTA), or 0.01 to 0.1% NTA by weight relative to the total weight of the detergent composition.

In one embodiment, the detergent composition of the invention comprises of at least one aminocarboxylate selected from methylglycine diacetate (MGDA), glutamic acid diacetate (GLDA), and the respective salts thereof, e.g., alkali (such as sodium) salts thereof in amounts in the range of 0.1% to 25.0% by weight, in the range of 1.0% to 18.0% by weight, in the range of 3.0% to 15.0% by weight, in the range of 3.0% to 10.0% by weight, or in the range of 5.0% to 8.0% by weight relative to the total weight of the detergent composition. Non-limiting examples of suitable salts of MGDA and/or GLDA include the trialkali metal salts of MGDA and GLDA such as the tripotassium salts and the trisodium salts.

In one embodiment of the present invention, alkali metal salts of MGDA are selected from compounds of the general formula (XIII):

[CH₃—CH(COO)—N(CH₂—COO)₂]Na_{3-x-y}KₓH_{y} (XIII)

The variables of formula (XIII) are defined as follows:

x is selected from 0.0 to 0.5, preferably up to 0.25,

y is selected from 0.0 to 0.5, preferably up to 0.25.

In one embodiment of the present invention, alkali metal salts of GLDA are selected from compounds of the general formula (XIV)

[OOC—(CH₂)₂—CH(COO)—N(CH₂—COO)₂]Na_{4-x-y}KₓH_{y} (XIV)

The variables of formula (XIV) are defined as follows:

x is selected from 0.0 to 0.5, preferably up to 0.25,

y is selected from 0.0 to 0.5, preferably up to 0.25.

In one embodiment of the present invention, alkali metal salts of MGDA may be selected from alkali metal salts of the L-enantiomer, of the racemic mixture and of enantiomerically enriched alkali metal salts of MGDA, with an excess of L-enantiomer compared to the D-enantiomer. Preference is given to alkali metal salts of mixtures from the L-enantiomer and the D-enantiomer in which the molar ratio of L/D is in the range of from 55:45 to 85:15. Such mixtures exhibit a lower hygroscopicity than, e.g., the racemic mixture. The enantiomeric excess can be determined, e.g., by measuring the polarization (polarimetry) or preferably by chromatography, for example by HPLC with a chiral column, for example with one or more cyclodextrins as immobilized phase. Preferred is determination of the enantiomeric excess by HPLC with an immobilized optically active ammonium salt such as D-penicillamine.

Alkali metal salts of GLDA may be selected from alkali metal salts of the L-enantiomer, of the racemic mixture and of enantiomerically enriched GLDA, with an excess of L-enantiomer compared to the D-enantiomer. Preference is given to alkali metal salts of mixtures from L-enantiomer and D-enantiomer in which the molar ratio of L/D is in the range of from 80:20 or higher, preferably of from 85:15 up to 99:1. Such alkali metal salts of GLDA have a better biodegradability than, e.g., the racemic mixture or the pure D-enantiomer. The enantiomeric excess can be determined, e.g., by measuring the polarization (polarimetry) or preferably by chromatography, for example by HPLC with a chiral column, for example with one or more cyclodextrins as immobilized phase. Preferred is determination of the enantiomeric excess by HPLC with an immobilized optically active ammonium salt such as D-penicillamine.

Generally, in the context of the present invention, small amounts of MGDA and/or GLDA may also bear a cation other than alkali metal. It is thus possible that small amounts of builder, such as 0.01% to 5 mol-% of total builder may bear alkali earth metal cations such as, e.g., Mg²⁺ or Ca²⁺, or a transition metal cation such as, e.g., a Fe²⁺ or Fe³⁺ cation. “Small amounts” of MGDA and/or GLDA herein refer to a total of 0.1% to 1 w/w %, relative to the respective builder. In one embodiment of the present invention, MGDA and/or GLDA comprised in detergent compositions may contain in the range of 0.1% to 10% by weight relative to the respective builder of one or more optically inactive impurities, at least one of the impurities being at least one of the impurities being selected from iminodiacetic acid, formic acid, glycolic acid, propionic acid, acetic acid and their respective alkali metal or mono-, di- or triammonium salts.

Detergent compositions of the invention may comprise one or more polycarboxylates. The term “polycarboxylates” includes polymeric polycarboxylates and non-polymeric polycarboxylates (non-polymeric polycarboxylates including compounds bearing two, three and four carbonic acid groups) such as succinic acid, C₂-C₁₆-alkyl disuccinates, C₂-C₁₆-alkenyl disuccinates, ethylene diamine N,N′-disuccinic acid, tartaric acid diacetate, alkali metal malonates, tartaric acid monoacetate, propanetricarboxylic acid, butanetetracarboxylic acid and cyclopentanetetracarboxylic acid.

Suitable polymeric polycarboxylates include compounds comprising monomers selected from unsaturated carboxylic acids of the general formula (XV):

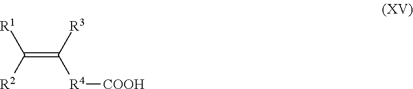

The variables in general formula (XV) are defined as follows:

R¹, R² and R³ are independently selected from H; linear or branched C₁-C₁₂ alkyl, linear or branched C₂-C₁₂ alkenyl, wherein alkyl and/or alkenyl may be substituted with —NH₂, —OH, or —COOH; —COOH; and —COOR⁵, wherein R⁵ is selected from linear or branched C₁-C₁₂ alkyl and linear or branched C₂-C₁₂ alkenyl.

R⁴ may be a spacer group, which is optionally selected from —(CH₂)ₙ— with n being in the range of 0 to 4, —COO—(CH₂)ₖ— with k being in the range of 1 to 6, —C(O)—NH— and —C(O)—NR⁶—, wherein R⁶ is selected from linear or branched C₁-C₂₂ alkyl, linear or branched C₂-C₂₂ alkenyl, and C₆-C₂₂ aryl.

Non-limiting examples of suitable unsaturated carboxylic acids include acrylic acid, methacrylic acid (MAA), 2-ethylacrylic acid, 2-phenylacrylic acid, malonic acid, crotonic acid, maleic acid (or maleic anhydride), fumaric acid, itaconic acid, aconitic acid, mesaconic acid, citraconic acid, sorbic acid, cinnamic acid, methylenemalonic acid, unsaturated C₄-C₁₀ dicarboxylic acids, and mixtures thereof.

Polycarboxylates may be homopolymers with the repeating monomer being the same unsaturated carboxylic acid, such as polyacrylic acid (PAA). Polycarboxylates may also be copolymers with the repeating monomers being at least two different unsaturated carboxylic acids, such as copolymers of acrylic acid with methacrylic acid, copolymers of acrylic acid or methacrylic acid and maleic acid and/or fumaric acid. In one embodiment, copolymers of acrylic acid and maleic acid comprise 50% to 90% by weight acrylic acid and 50% to 10% by weight maleic acid.

Polycarboxylates may also be copolymers with at least one monomer from the group consisting of monoethylenically unsaturated carboxylic acids as defined above, with at least one hydrophobically or hydrophilically modified monomer. Suitable hydrophobic monomers are, for example, isobutene, diisobutene, butene, pentene, hexene and styrene, olefins with 10 or more carbon atoms or mixtures thereof, such as, for example, 1-decene, 1-dodecene, 1-tetradecene, 1-hexadecene, 1-octadecene, 1-eicosene, 1-docosene, 1-tetracosene and 1-hexacosene, C₂₂-α-olefin, a mixture of C₂₀-C₂₄-α-olefins and polyisobutene having on average 12 to 100 carbon atoms per molecule.

Suitable hydrophilic monomers are monomers with sulfonate or phosphonate groups, and also non-ionic monomers with hydroxyl function or alkylene oxide groups. By way of example, mention may be made of: allyl alcohol, isoprenol, methoxypolyethylene glycol (meth)acrylate, methoxypolypropylene glycol (meth)acrylate, methoxypolybutylene glycol (meth)acrylate, methoxy-poly(propylene oxide-co-ethylene oxide) (meth)acrylate, ethoxypolyethylene glycol (meth)acrylate, ethoxypolypropylene glycol (meth)acrylate, ethoxypolybutylene glycol (meth)acrylate and ethoxypoly (propylene oxide-co-ethylene oxide) (meth)acrylate. Polyalkylene glycols here may comprise 3 alkylene oxide units (AO) to 50 AO per molecule, 5 AO to 40 AO per molecule, or 10 AO to 30 AO per molecule.

Polycarboxylates include salts of the compounds listed above. Salt forming cations may be monovalent or multivalent. Suitable examples include but are not limited to sodium, potassium, magnesium, calcium, ammonium, and the ammonium salt of mono-, di- and triethanolamine.

Suitable polycarboxylates according to the invention include polycarboxylate compounds which have average molecular weights (Mw) in the range of about 500 g/mol to about 500,000 g/mol, in the range of about 1,000 g/mol to about 100,000 g/mol, or in the range of about 3,000 g/mol to about 80,000 g/mol.

Polycarboxylates may be derivatized by alkoxylation such as ethoxylation and/or propoxylation.

Alkoxylated polycarboxylates comprise polyacrylates having one ethoxy side-chain per every 2 to 8 acrylate units. In one embodiment alkoxylated polycarboxylates comprise polyacrylates having one ethoxy side-chain per every 7 to 8 acrylate units. The side-chains are ester-linked to the polyacrylate “backbone” to provide a “comb” polymer type structure. The molecular weight may be in the range of about 2,000 g/mol to about 50,000 g/mol.

Suitable, non-limiting examples of polycarboxylates comprising acrylic acid include Sokalan PA30, Sokalan PA20, Sokalan PA15, Sokalan PAIO and Sokalan CP10 (BASF GmbH, Ludwigshafen, Germany), Acusol™ 45N, Acusol 480N, Acusol 460N and Acusol 820 (sold by Rohm and Haas, Philadelphia, Pa., USA) polyacrylic acids, such as Acusol™ 445 and Acusol™ 420 (sold by Rohm and Haas, Philadelphia, Pa., USA) acrylic/maleic co-polymers, such as Acusol™ 425N and acrylic/methacrylic copolymers.

The detergent compositions described herein may comprise amounts of alkoxylated polycarboxylates in the range of 0.1% to 10% w/w, 0.25% to 5% w/w, or 0.3% to 2% w/w of the detergent composition.

Detergent compositions may comprise polymers selected from the group of polysulfonates.

“Polysulfonates” include compounds comprising sulfonic acid monomers of the general formula (XVI):

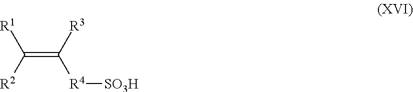

wherein the variables in formula (XVI) are defined as follows:

R¹, R² and R³ are independently selected from H; linear or branched C₁-C₁₂ alkyl, linear or branched C₂-C₁₂ alkenyl, wherein alkyl and/or alkenyl may be substituted with —NH₂, —OH, or —COOH; —COOH; and —COOR⁵, wherein R⁵ is selected from linear or branched C₁-C₁₂ alkyl and linear or branched C₂-C₁₂ alkenyl.

R⁴ may be a spacer group, which is optionally selected from —(CH₂)ₙ— with n being in the range of 0 to 4, —COO—(CH₂)ₖ— with k being in the range of 1 to 6, —C(O)—NH— and —C(O)—NR⁶—, wherein R⁶ is selected from linear or branched C₁-C₂₂ alkyl, linear or branched C₂-C₂₂ alkenyl, and C₆-C₂₂ aryl (the latter meant to include also annulated ring systems of more than one ring selected from 5, 6, 7, and 8-membered rings, such as naphthalene).

In one embodiment, the sulfonic acid monomers are selected from compounds according to formulae (XVII), (XVIII), and (XIX):

H₂C═CH—X—SO₃H (XVII)

H₂C═C(CH₃)—X—SO₃H (XVIII)

HO₃S—X—(R²)C═C(R³)—X—SO₃H (XIX)

The variables in formulae (XVII), (XVIII), and (XIX) are defined as follows:

R² and R³ are independently selected from H, methyl, ethyl, propyl and iso-propyl.

X may be a spacer group, which is optionally selected from —(CH₂)ₙ— with n being in the range of 0 to 4, —COO—(CH₂)ₖ— with k being in the range of 1 to 6, —C(O)—NH— and —C(O)—NR⁵—, wherein R5 is selected from linear or branched C₁-C₂₂ alkyl, linear or branched C₂-C₂₂ alkenyl, and C₆-C₂₂ aryl.

Non-limiting examples of suitable sulfonic acid monomers include, 1-acrylamido-1-propane sulfonic acid, 2-acrylamido-2-propane sulfonic acid, 2-acrylamido-2-methyl-1-propane sulfonic acid, 2-methacrylamido-2-methyl-1-propane sulfonic acid, 3-methacrylamido-2-hydroxy-1-propane sulfonic acid, allylsulfonic acid, methallylsulfonic acid, allyloxybenzene sulfonic acid, methallyloxybenzene sulfonic acid, 2-hydroxy-3-(2-propenyloxy)-propane sulfonic acid, 2-methyl-2-propene-sulfonic acid, styrene sulfonic acid, vinylsulfonic acid, 3-sulfopropylacrylate, 3-sulfopropylmethacrylate, sulfomethacrylamide, sulfomethylmetharylamide, and mixtures thereof.

In one embodiment, polysulfonates comprise sulfonic acid monomers as well as monomers selected from unsaturated carboxylic acids. Monomers selected from unsaturated carboxylic acids include those listed as suitable monomers for polycarboxylates.

Polysulfonates include salts of the compounds listed above. Salt forming cations may be monovalent or multivalent. Suitable examples include but are not limited to sodium, potassium, magnesium, calcium, ammonium, and the ammonium salt of mono-, di- and triethanolamine.

Suitable polysulfonates may have a weight average molecular weight of less than or equal to about 100,000 g/mol, of less than or equal to about 75,000 g/mol, or of less than or equal to about 50,000 g/mol. Suitable polysulfonates may have a weight average molecular weight in the range of about 3,000 g/mol to about 50,000 g/mol, or in the range of about 5,000 g/mol to about 45,000 g/mol.

Suitable, non-limiting examples for sulfonated/carboxylated polymers include Alcosperse 240, Aquatreat AR 540 and Aquatreat MPS supplied by Alco Chemical; Acumer 3100, Acumer 2000, Acusol 587G and Acusol 588G supplied by Rohm & Haas; Goodrich K-798, K-775 and K-797 supplied by BF Goodrich; and ACP 1042 supplied by ISP technologies Inc. Particularly preferred polymers are Acusol 587G and Acusol 588G supplied by Rohm & Haas, Versaflex Si™ (sold by Alco Chemical, Tennessee, USA).

Detergent compositions of the invention may comprise one or more polyphosphonates. The term “polyphosphonates” includes copolymers of vinylphosphonic acid and acrylic acid or a further vinyl compound, polyvinylphosphonic acid, and salts thereof. Salt forming cations may be monovalent or multivalent. Suitable examples include but are not limited to sodium, potassium, magnesium, calcium, ammonium, and the ammonium salt of mono-, di- and triethanolamine.

Detergent compositions may comprise one or more polyamines. “Polyamines” are compounds which may be selected from the group consisting of:

i) polyamines comprising two or more backbone nitrogen atoms;

ii) polyamines comprising one or more cationic backbone nitrogen atoms;

iii) polyamines comprising one or more alkoxylated backbone nitrogen atoms;

iv) polyamines comprising one or more cationic backbone nitrogen atoms and one or more alkoxylated backbone nitrogen atoms; and

v) mixtures thereof.

The polyamines comprise a polyamine backbone wherein the backbone units which connect the amino units can be modified.

In addition to modification of the backbone compositions, one or more of the backbone amino unit hydrogens may be substituted by other units, which may introduce an anionic or cationic moiety into the polyamine.

In the context of polyamines, “cationic moieties” are defined as “units which are capable of having a positive charge”. Such cationic units may be quaternary ammonium units of the polyamine backbones (i.e. amino groups within the polyamine backbone that are modified to become ammonium units) or quaternary ammonium units which comprise the units which substitute the polyamine backbone.

In the context of polyamines, “anionic moieties” are defined as “units which are capable of having a negative charge”. Such anionic units are “units which alone, or as a part of another unit, substitute for hydrogens along the polyamine backbone”.

In one embodiment, polyamines according to the invention are polyalkylene imines having a basic skeleton, i.e. polyamine backbone, which comprises primary, secondary, and tertiary amine nitrogen atoms J which are joined by alkylene radicals R to form compounds of the general formula [J-R]ₙ-J.

The R units may be selected from the group of
a) C₂-C₁₂ linear alkylene, C₃-C₁₂ branched alkylene, C₆-C₁₆ substituted or unsubstituted arylene, C₇-C₄₀ substituted or unsubstituted alkylenearylene or mixtured thereof.
b) Alkyleneoxyalkylene units according to formula —(R²O)_{w} R³—,
   wherein R² is selected from the group consisting of ethylene, 1,2-propylene, 1,3-propylene, 1,2-butylene, 1,4-butylene, and mixtures thereof;
   and wherein R³ is selected from the group consisting of C₂-C₈ linear alkylene, C₃-C₈ branched alkylene, phenylene, substituted phenylene, and mixtures thereof.
   The index w is in the range of 0 to about 25.
   R² and R³ units may also comprise other backbone units. When comprising alkyleneoxyalkylene units R² and R³ units are preferably mixtures of ethylene, propylene and butylene and the index w may be in the range of 1 to about 20, in the range of about 2 to about 10, or in a range to about 6.
c) hydroxyalkylene units according to formula

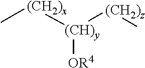

wherein R⁴ is hydrogen, C₁-C₆ alkyl, —(CH₂)ᵤ(R²O)ₜ(CH₂)ᵤY, and mixtures thereof. When R units comprise hydroxyalkylene units, R⁴ may be hydrogen or —(CH₂)ᵤ(R²O)ₜ(CH₂)ᵤY, wherein the index t is greater than 0, e.g. in the range of 10 to 30; the index u may be in the range of 0 to 6; and Y may be hydrogen or an anionic unit, such as —SO₃M; x, y, and z are each independently in the range of 0 to 20. In one embodiment, the indices are each at least 1 and R⁴ is hydrogen (2-hydroxypropylene unit) or (R²O)ₜY. For polyhydroxy units y is selected from 2 and 3.
d) hydroxyalkylene/oxyalkylene units according to formula:

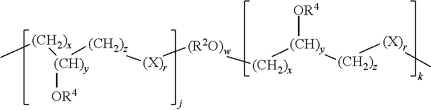

wherein R² is selected from the group consisting of ethylene, 1,2-propylene, 1,3-propylene, 1,2-butylene, 1,4-butylene, and mixtures thereof (as described under b)); R⁴ is hydrogen, C₁-C₆ alkyl, —(CH₂)ᵤ(R²O)ₜ(CH₂)ᵤY, and mixtures thereof (as described under c)); w is in the range of 0 to about 25 (as described under d)); x, y, and z are each independently in the range of 0 to 20 (as described under c)). X may be oxygen or the amino unit —NR⁴—, the index r may be 0 or 1. The indices j and k are each independently in the range of 1 to 20. When alkyleneoxy units are absent the index w is 0.
e) carboxyalkyleneoxy units according to formula:

—(R²O)_{w}(R³)_{w}(X)ᵣ—CO—(X)ᵣ—R³—(X)ᵣ—CO—(X)ᵣ(R³)_{w}(OR²)_{w}—
wherein R² is selected from the group consisting of ethylene, 1,2-propylene, 1,3-propylene, 1,2-butylene, 1,4-butylene, and mixtures thereof (as described under b)); R³ is selected from the group consisting of C₂-C₈ linear alkylene, C₃-C₈ branched alkylene, phenylene, substituted phenylene, and mixtures thereof (as described under b)); w is in the range of 0 to about 25 (as described under b)); X may be oxygen or the amino unit —NR⁴— (as described under d)); r may be 0 or 1 (as described under d)).
f) backbone branching units according to formula

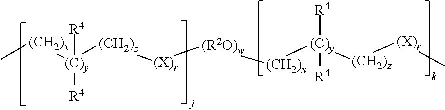

wherein R⁴ is hydrogen, C₁-C₆ alkyl, —(CH₂)ᵤ(R²O)ₜ(CH₂)ᵤY, and mixtures thereof (as described under c)). When R units comprise backbone branching units, R⁴ may be hydrogen or —(CH₂)ᵤ(R²O)ₜ(CH₂)ᵤY. j and k are each independently in the range of 1 to 20 (as described under d)); t 0, e.g. in the range of 10 to 30 and u may be in the range of 0 to 6 (as described under c)); w is in the range of 0 to about 25 (as described under d)); x, y, and z are each independently in the range of 0 to 20 (as described under c)). Y may be hydrogen, C₁-C₄ linear alkyl, —N(R¹)₂, an anionic unit, or mixtures thereof.

The R units disclosed may be combined with each other to achieve various degrees of hydrophilicity of the polyamine.

In the context of polyamines, the term “substitutent(s)” is defined as “compatible moieties which replace a hydrogen atom”. Non-limiting examples of suitable substituents include hydroxy, nitrilo, oximino, halogen, nitro, carboxyl, and inter alia —CHO, CO₂H, —CO₂R′, —CONH₂, —CONHR′, —CONR′₂, wherein R′ is C₁-C₁₂ linear or branched alkyl, amino, C₁-C₁₂ mono- or di-alkylamino, —OSO₃M, —SO₃M, —OPO₃M, or —OR″, wherein R″ is C₁-C₁₂ linear or branched alkyl; and mixtures thereof.

M is selected from H, salt forming cations such as Na, and mixtures thereof.

The J units are the backbone amino units, said units are selected from the group consisting of:
i) primary amino units having the formula: [NH₂—R¹]— and —NH₂
ii) secondary amino units having the formula: —[NH—R¹]—
iii) tertiary amino units having the formula: —[NB—R¹]—
iv) primary quaternary amino units having the formula: —[N⁺H₂—R¹]—
v) secondary quaternary amino units having the formula: —[N⁺H(Q)—R¹]—
vi) tertiary quaternary amino units having the formula: —[N⁺B(Q)—R¹]—
vii) primary N-oxide amino units having the formula: —[NH₂(O)—R¹]—
viii) secondary N-oxide amino units having the formula: —[NH(O)—R¹]—
ix) tertiary N-oxide amino units having the formula: —[NB(O)—R¹]—
x) and mixtures thereof.

The B units comprised in aforementioned J units have the formula [J-R]— and represent a continuation of the polyamine backbone by branching. The number of B units present, as well as any further amino units which comprise the branches are reflected in the total value of the index n.

The R¹ units in aforementioned J units may be selected from
a. hydrogen (which is typically present prior to any backbone modification)
b. C₁-C₂₂ alkyl, C₁-C₄ alkyl, ethyl, and methyl
c. quaternizing unit Q
d. C₇-C₂₂ arylenealkyl according to one of the following formulae:

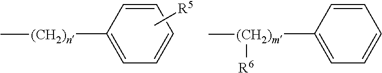

wherein R⁵ may be linear or branched C₁-C₁₆ alkyl and n′ may be 0 or 1;
wherein R⁶ may be hydrogen, linear or branched C₁-C₁₅ alkyl, and mixture thereof; m′ may be in the range of 1 to 16.
e. —[CH₂CH(OR⁴)CH₂O]ₛ(R²O)ₜY
   wherein R² is selected from the group consisting of ethylene, 1,2-propylene, 1,3-propylene, 1,2-butylene, 1,4-butylene, and mixtures thereof;
   R⁴ may be hydrogen, C₁-C₆ alkyl, —(CH₂)ᵤ(R²O)ₜ(CH₂)ᵤY, or mixtures thereof wherein the index t is greater than 0, e.g. in the range of 10 to 30; the index u may be in the range of 0 to 6; and Y may be hydrogen C₁-C₄ linear alkyl, —N(R¹)₂, or an anionic unit; Y may be —N(R¹)₂ when Y is part of an R unit which is a backbone branching unit;
   the index s may be in the range of 0 to 5. Index t is an average value in the range of 0.5 to about 100, or in the range of 5 to about 15.
f. anionic units.

The Q unit in aforementioned J units is a quaternizing unit selected from the group consisting of C₁-C₄ linear alkyl (such as methyl), benzyl, and mixtures thereof. For each backbone quaternary nitrogen there will be an anion to provide charge neutrality.

The anionic groups include both units which are covalently attached to the polymer as well as external anions which are present to achieve charge neutrality. Non-limiting examples of anions suitable for use include halogen, inter alia, chloride; methyl sulfate; hydrogen sulfate, and sulfate. The one skilled in the art will recognize that the anion will typically be a unit which is part of the quaternizing reagent, inter alia, methyl chloride, dimethyl sulfate, benzyl bromide.

For example, a carboxylic acid unit, —CO₂H, is neutral, however upon de-protonation the unit becomes an anionic unit. Non-limiting examples of anionic Y units include —(CH₂)_{f}CO₂M, —C(O)(CH₂)_{f}CO₂M, —(CH₂)_{f}PO₃M, —(CH₂)_{f}OPO₃M, —(CH₂)_{f}SO₃M, —CH₂(CHSO₃M)-(CH₂)_{f}SO₃M, —CH₂(CHSO₂M)(CH₂)_{f}SO₃M, —C(O)CH₂CH(SO₃M)CO₂M, —C(O)CH₂CH(CO₂M)NHCH(CO₂M)CH₂CO₂M, —C(O)CH₂CH(CO₂M)NHCH₂CO₂M, —CH₂CH(OZ)CH₂O(R¹O)ₜZ, —(CH₂)_{f}CH—[O(R²O)ₜZ]CH_{f}O(R²O)ₜZ, and mixtures thereof; wherein Z is hydrogen or an anionic unit; f is in the range of 0 to 6.

Anionic Y units further include oligomeric and polymeric units of the formulae

—CH₂CH(OH)CH₂O—CH₂CH(SO₃Na)CH₂SO₃Na

—CH₂CH(OH)CH₂O—CH₂CH(SO₂Na)CH₂SO₃Na

—CH₂CH(OH)CH₂O—CH₂CH₂CH₂SO₃Na

—CH₂CH(OSO₃Na)CH₂O—CH₂CH(SO₂Na)CH₂OSO₃Na

Polyamines may comprise one or more anionic units which are substituted on the polyamine backbone.

Usually, granular detergent compositions require a high degree of anionic charge, which means that about 40%, more than 50%, more than 75%, or more than 90% of anionic Y units may comprise —SO₃M units.

Usually liquid detergent compositions require less than 90%, less than 75%, less than 50% or less than 40% of anionic Y units comprising —SO₃M.

The polyamine compounds may comprise a polyamine backbone of the following formula:

[H₂N—R]_{w}[N(H)—R]ₓ[N(B)—R]_{y} NH₂

wherein R is C₂-C₁₂ linear alkylene, C₃-C₁₂ branched alkylene, and mixtures thereof; B represents a continuation of the structure by branching; w, x and y vary depending on molecular weight and relative degree of branching.

Low molecular weight polyalkyleneimines may have R selected from ethylene, 1,3-popylene and 1,6 hexylene. The indices w, x and y are such that the molecular weight of said low molecular polyalkyleneimines does not exceed 600 g/mol. Non-limiting examples polyamine units in low molecular weight polyalkyleneimines include diethylene triamine, triethylene tetramine, tetra ethylene pentamine, dipropylene triamine, tripropylene tetramine, and dihexamethylene triamine.

Medium range molecular weight polyalkyleneimines may have R selected from ethylene, 1,3-propylene, and mixtures thereof. The indices w, x, and y are such that the molecular weight of said polyamines is in the range of about 600 g/mol to about 50,000 g/mol.

High molecular weight polyalkyleneimines may have R selected from ethylene. The indices w, x, and y are such that the molecular weight of said polyamines is in the range of about 50,000 g/mol to about 1,000,000 g/mol.

Polyalkyleneimines may have a range of average molecular weight (M_{w}) of about 100 g/mol up to several million g/mol. Preferably, average molecular weights are in the range of about 100 g/mol to about 1,000,000 g/mol, in the range of about 250 g/mol to 100,000 g/mol, in the range of about 500 g/mol to about 5,000 g/mol, in the range of about 500 g/mol to about 1,000 g/mol, or in the range of about 600 g/mol to about 800 g/mol.

Polyalkyleneimines may be linear or branched and may further be modified by grafting or capping. Non-limiting examples of preferred grafting agents are aziridine (ethyleneimine), caprolactam, and mixtures thereof. Suitable capping reactions include but are not limited to reaction of polyamine with C₁-C₂₂ linear or branched monocarboxylic acid, such as lauric acid and myristic acid.

Prior or after grafting, polyamines may be crosslinked with amide forming T crosslinking units which may be carbonyl comprising polyamido forming units or with non-amide forming L cross-linking units which may be derived from the use of epihalohydrins, preferably epichlorohydrin, as a crosslinking agent.

Preferred polyakyleneimine backbones herein are those that exhibit little or no branching, thus predominantly linear polyalkylenimine backbones. In the context of the present invention, CH₃-groups in polyalkyleneimines are not being considered as branches. Branches may be alkylenamino groups such as, but not limited to —CH₂—CH₂—NH₂ groups or (CH₂)₃—NH₂-groups. Longer branches may be, for examples, —(CH₂)₃—N(CH₂CH₂CH₂NH₂)₂ groups.

Detergent compositions of the invention may one or more pH-adjusting compounds, which may be called alkalines herein, providing a pH above 5, above 6, or above 7. Preferably, pH-adjusting compounds provide a pH above 7.5, above 8, above 8.5, above 9, above 9.5, above 10, above 10.5, above 11, or above 11.5 when added to the detergent composition.

In one embodiment, the inventive composition comprises a pH-adjusting compound providing a pH of the liquid composition in the range of 5 to 11.5, in the range of 6 to 11.5, in the range of 7 to 11, or in the range of 8 to 11.

Suitable pH-adjusting compounds may be sodium hydroxide, potassium hydroxide, ethanol amine and/or alkaline buffer salts. Suitable buffer salts may be potassium bicarbonate, potassium carbonate, tetra potassium pyrophosphate, potassium tripolyphosphate, sodium bicarbonate and sodium carbonate. Suitable might also be mixtures of pH-adjusting compounds which satisfy the purpose of adjusting the appropriate pH.

Detergent compositions of the invention may be adapted in sudsing characteristics for satisfying various purposes. Hand dishwashing detergents usually request stable suds. Automatic dishwasher detergents are usually requested to be low sudsing. Laundry detergents may range from high sudsing through a moderate or intermediate range to low. Low sudsing laundry detergents are usually recommended for front-loading, tumbler-type washers and washer-dryer combinations.

Those skilled in the art are familiar with using suds stabilizers or suds suppressors as detergent components in detergent compositions which are suitable for specific applications. Suitable suds stabilizers may be selected from alkanolamides and alkylamine oxides. Suitable suds suppressors may be selected from alkyl phosphates, silicones and soaps.

Depending on the final application, detergent compositions of the invention may comprise one or more anti-redeposition agents, which may be called anti-greying agents herein. Usually anti-redeposition agents are meant to prevent soil from resettling after removal during cleaning. Non-limiting examples of suitable anti-redeposition agents include carboxymethyl cellulose, polycarbonates, polyethylene glycol and sodium silicate.

Depending on the final application, detergent compositions of the invention may comprise one or more dye-transfer inhibition agents (DTI). Usually dye-transfer inhibition agents are meant to prevent dyes released from one textile to be transferred to another textile present during laundering. Non-limiting examples of suitable dye transfer inhibiting agents include modified polycarboxylates, polyamine N-oxides such as poly(4-vinylpyridine-N-oxide), such as PVNO and copolymers of N-vinylpyrrolidone and N-vinylimidazole, such as PVPVI.

Depending on the final application, detergent compositions may comprise one or more bleaching agents, like chlorine bleaches, photobleaches, and peroxide bleaches, as well as mixtures thereof. Peroxide bleaches may be combined with bleach activators and/or bleach catalysts. Non-limiting examples of suitable chlorine bleaches include but are not limited to 1,3-dichloro-5,5-dimethylhydantoin, N-chlorosulfamide, chloramine T, chloramine B, sodium hypochlorite, calcium hypochlorite, magnesium hypochlorite, potassium hypochlorite, potassium dichloroisocyanurate, and sodium dichloroisocyanurate.

Non-limiting examples of suitable photobleaches include sulfonated zinc phthalocyanines and sulfonated aluminium phthalocyanines, as well as mixtures thereof.

Detergent compositions according to the invention may comprise one or more peroxide bleaches. Peroxide bleaches may be selected from H₂O₂ and precursors of H₂O₂. Suitable examples of precursors of H₂O₂ include compounds such as inorganic and organic peroxides, and peroxy acids. Inorganic peroxides may be selected from compounds of the group of persulfates, perborates, percarbonates, and persilicates. Non-limiting examples of suitable inorganic peroxides are sodium perborate tetrahydrate, sodium perborate monohydrate and sodium percarbonate. Organic peroxides may be selected from compounds of the group of mono- or poly-peroxides, urea peroxides, a combination of a C₁-C₄ alkanol oxidase and C₁-C₄ alkanol, alkylhydroxy peroxides (e.g. cumene hydroperoxide), and t-butyl hydroperoxide.

The peroxides comprised in the detergent compositions of the invention may be in a variety of different crystalline forms and have different water contents, and they may also be used together with other inorganic or organic compounds in order to improve their storage stability. Peroxy acids may be selected from inorganic and organic peroxy acids. A suitable, non-limiting example for an inorganic peroxy acid is potassium monopersulphate (MPS). Organic peroxy acids may be selected from organic mono peroxy acids of the formula (XX):

R′—C(O)—O—OM′ (XX)

wherein M′ is hydrogen or an alkali metal (e.g. Na-salts), and

R′ is hydrogen, C₁-C₄ alkyl, phenyl, —C₁-C₂ alkylene-phenyl or phthalimido-C₁-C₈ alkylene.

Non-limiting examples of suitable peroxy acids according to formula (XX) include HCOOOH, CH₃COOOH, epsilon-phthalimido peroxy hexanoic acid, and their alkali salts (e.g. Na-salts).

Peroxy acids may be selected from diperoxy acids, such as 1,12-diperoxydodecanedioic acid (DPDA); 1,9-diperoxyazelaic acid; diperoxybrassilic acid; diperoxysebasic acid; diperoxyisophthalic acid; 2-decyldiperoxybutane-1,4-diotic acid; 4,4′-sulphonylbisperoxybenzoic acid; magnesium bis(monoperoxyphthalate) hexahydrate (Mg-DPP); dinonanoyl peroxide (DAP); and peroxybenzoic acid.

In one embodiment, detergent compositions according to the invention comprise one or more inorganic peroxides.

The peroxides, especially the inorganic peroxides, can optionally be activated by a bleach activator. Therefore, detergent compositions of the invention may comprise one or more bleach activators. Such bleach activators may, under perhydrolysis conditions, yield unsubstituted or substituted perbenzo- and/or peroxo-carboxylic acids having 1 to 10 carbon atoms, or 2 to 4 carbon atoms. Non-limiting examples of suitable bleach activators include those that carry O- and/or N-acyl groups having said number of carbon atoms and/or unsubstituted or substituted benzoyl groups. Preference may be given to compound selected from polyacylated alkylenediamines such as tetraacetyl ethylenediamine (TAED), acylated glycolurils such as tetraacetylglycoluril (TAGU), N,N-diacetyl-N,N-dimethyl-urea (DDU), acylated triazine derivatives such as 1,5-diacetyl-2,4-dioxohexahydro-1,3,5-triazine (DADHT), and compounds of formula (XXI)

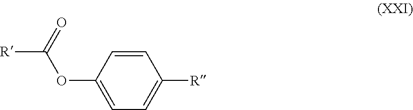

wherein the variables in formula (XXI) are defined as follows:

R″ is a sulfonate group, a carboxylic acid group or a carboxylate group,

R′ is linear or branched (C₇-C₁₅) alkyl.

Non-limiting examples of suitable bleach activators include compounds that are known under the names LOBS (dodecanoyloxy benzene sulfonate), NOBS (nonanoyloxy benzene sulfonate), IsoNOBS (Na 3,5,5-trimethylhexanoyloxybenzene sulfonate) and DOBA (decanoyloxy benzoic acid), BOBS (benzoyloxy benzene sulfonate), BCL (benzoyl caprolactam), MOR (4-Morpholinocarbonitrile), and ACL (acetyl caprolactam).

Suitable bleach activators may also be selected from alkanoyloxyethanoate compounds, acylated polyhydric alcohols such as especially triacetin, ethylene glycol diacetate, 2,5-diacetoxy-2,5-dihydrofuran, acetylated sorbitol and mannitol. Suitable bleach activators may also be selected from acylated sugar derivatives such as pentaacetylglucose (PAG), sucrose polyacetate (SUPA), pentaacetylfructose, tetraacetylxylose, and octaacetyllactose. Suitable bleach activators may also be selected from acetylated, optionally N-alkylated, glucamine and gluconolactone.

Nitrile compounds that form peroxyimidic acids with peroxides may also be suitable as bleach activators.

In one embodiment tetraacetyl ethylenediamine and/or nonanoyloxy benzene sulfonate are comprised in detergent compositions of the invention.

The peroxides may also be used in combination with a bleach catalyst and optionally in combination with a bleach activator. Bleach catalysts may be selected from oxaziridinium-based bleach catalysts, from acylhydrazone bleach catalysts, bleach-boosting transition metal salts or transition metal complexes such as, for example, manganese-, iron-, cobalt-, ruthenium- or molybdenum-salen complexes or carbonyl complexes. Manganese, iron, cobalt, ruthenium, molybdenum, titanium, vanadium and copper complexes with nitrogen-containing tripod ligands may be used as bleach catalysts.

Non-limiting examples of bleach catalysts that may be used include manganese oxalate, manganese acetate, manganese-collagen, cobalt-amine catalysts, terpyridine-manganese complexes and manganese triazacyclononane (MnTACN) catalysts; suitable are complexes of manganese with 1,4,7-trimethyl-1,4,7-triazacyclononane (Me₃-TACN), or 1,2,4,7-tetramethyl-1,4,7-triazacyclononane (Mea-TACN), in particular Me₃-TACN, such as the dinuclear manganese complex [(Me₃-TACN)Mn(O)3Mn(Me₃-TACN)](PF₆)₂, and [2,2′,2″-nitrilotris(ethane-1,2-diylazanylylidene-κN-methanylylidene)triphenolato-κ3O]manganese(III), Fe(III)TAML and Pentamminacetatocobalt (III) nitrat (PAAN). The bleach catalysts may also be other metal compounds, such as cobalt-, iron-, copper- and ruthenium-amine complexes.

Further examples of bleach catalysts are N-sulfonyloxaziridine, sufonimines, quarternary imine salts, quaternary oxazridinium salts, dihydroisoquinoliumium compounds, quaternary oxaziridinium compounds and precursors thereof.

Depending on the final application, detergent compositions may comprise one or more fluorescent whitening agents (FWA). Detergent compositions may comprise fluorescent whitening agents selected from compounds of the classes of bis-triazinylamino-stilbenedisulphonic acids, such as Tinopal® DMA-X and Tinopal® 5BM-GX.

Fluorescent whitening agents may also be selected from compounds of the classes bis-triazolyl-stilbenedisulphonic acids, and bis-styryl-biphenyl derivative such as Tinopal® CBS-X, CBS-SP, and CBS-CL.

Fluorescent whitening agents may also be selected from compounds of the classes bis-benzofuranylbiphenyls, bis-benzoxalyl derivatives, bis-benzimidazolyl derivatives, coumarin derivatives, naphtha-triazole-stilbene derivatives, pyrazoline derivatives, and bis-styrylbenzenes.

Non-limiting examples of suitable fluorescent whitening agents also include 4,4′-bis-(2-diethanolamino-4-anilino-s-triazin-6-ylamino) stilbene-2,2′-disulphonate; 4,4′-bis-(2,4-dianilino-s-triazin-6-ylamino) stilbene-2.2′-disulphonate; 4,4′-bis-(2-anilino-4(N-methyl-N-2-hydroxyethylamino)-s-triazin-6-ylamino) stilbene-2,2′-disulphonate; 4,4′-bis-(2-anilino-4-(methylamino)-s-triazin-6-ylamino) stilbene-2,2′-disulphonate; 4,4′-bis-(4-phenyl-2,1,3-triazol-2-yl)stilbene-2,2′-disulphonate; 2-(stilbyl-4″)-(naphtho-1′,2′:4,5)-1,2,3-triazole-2″-sulphonate; 4,4′-bis{[(4-anilino-6-morpholino-1,3,5-triazin-2-yl)]amino} stilbene-2-2′-disulphonate; and 4,4′-bis(2-sulfostyryl) biphenyl.

Depending on the physical form, detergent compositions of the invention may comprise one or more preservatives. Preservatives are usually added to liquid compositions to prevent alterations of said compositions due to attacks from microorganisms. Non-limiting examples of suitable preservatives include (quarternary) ammonium compounds, isothiazolinones, organic acids, and formaldehyde releasing agents. Non-limiting examples of suitable (quaternary) ammonium compounds include benzalkonium chlorides, polyhexamethylene biguanide (PHMB), Didecyldimethylammonium chloride(DDAC), and N-(3-aminopropyl)-N-dodecylpropane-1,3-diamine (Diamine). Non-limiting examples of suitable isothiazolinones include 1,2-benzisothiazolin-3-one (BIT), 2-methyl-2H-isothiazol-3-one (MIT), 5-chloro-2-methyl-2H-isothiazol-3-one (CIT), 2-octyl-2H-isothiazol-3-one (OIT), and 2-butyl-benzo[d]isothiazol-3-one (BBIT). Non-limiting examples of suitable organic acids include benzoic acid, sorbic acid, L-(+)-lactic acid, formic acid, and salicylic acid. Non-limiting examples of suitable formaldehyde releasing agent include N,N′-methylenebismorpholine (MBM), 2,2′,2″-(hexahydro-1,3,5-triazine-1,3,5-triyl)triethanol (HHT), (ethylenedioxy)dimethanol, .alpha.,.alpha.′,.alpha.″-trimethyl-1,3,5-triazine-1,3,5(2H,4H,6H)-triethanol (HPT), 3,3′-methylenebis[5-methyloxazolidine] (MBO), and cis-1-(3-chloroallyl)-3,5,7-triaza-1-azoniaadamantane chloride (CTAC).

Further useful preservatives include iodopropynyl butylcarbamate (IPBC), halogen releasing compounds such as dichloro-dimethyl-hydantoine (DCDMH), bromo-chloro-dimethyl-hydantoine (BCDMH), and dibromo-dimethyl-hydantoine (DBDMH); bromo-nitro compounds such as Bronopol (2-bromo-2-nitropropane-1,3-diol), 2,2-dibromo-2-cyanoacetamide (DBNPA); aldehydes such as glutaraldehyde; phenoxyethanol; Biphenyl-2-ol; and zinc or sodium pyrithione.

Depending on the physical form, detergent compositions of the invention may comprise one or more rheology modifiers, which may be called thickener herein. “Thickener(s)” according to the invention are selected from the following:

i.) Polymeric Structuring Agents

Examples of naturally derived polymeric structurants of use in the present invention include: hydroxyethyl cellulose, hydrophobically modified hydroxyethyl cellulose, carboxymethyl cellulose, polysaccharide derivatives and mixtures thereof. Suitable polysaccharide derivatives include: pectine, alginate, arabinogalactan (gum Arabic), carrageenan, gellan gum, xanthan gum, guar gum and mixtures thereof. Examples of synthetic polymeric structurants of use in the present invention include: polycarboxylates, polyacrylates, hydrophobically modified ethoxylated urethanes, hydrophobically modified non-ionic polyols and mixtures thereof. In one aspect, said polycarboxylate polymer may be a polyacrylate, polymethacrylate or mixtures thereof. In another aspect, the polyacrylate may be a copolymer of unsaturated mono- or di-carbonic acid and C₁-C₃₀ alkyl ester of the (meth)acrylic acid. Said copolymers are available from Noveon inc under the tradename Carbopol Aqua 30.

ii.) Di-benzylidene Polyol Acetal Derivative

A composition according to the invention may comprise one or more dibenzylidene polyol acetal derivatives (DBPA). The DBPA derivative may comprise a dibenzylidene sorbitol acetal derivative (DBS). Said DBS derivative may be selected from the group consisting of: 1,3:2,4-dibenzylidene sorbitol; 1,3:2,4-di(p-methylbenzylidene) sorbitol; 1,3:2,4-di(p-chlorobenzylidene) sorbitol; 1,3:2,4-di(2,4-dimethyldibenzylidene) sorbitol; 1,3:2,4-di(p-ethy (benzylidene) sorbitol; 1,3:2,4-di(3,4-dimethyldibenzylidene) sorbitol; and mixtures thereof.

iii.) Di-amido-gellants

In one aspect, the external structuring system may comprise a di-amido gellant having a molecular weight from about 150 g/mol to about 1,500 g/mol, or even from about 500 g/mol to about 900 g/mol. Such di-amido gellants may comprise at least two nitrogen atoms, wherein at least two of said nitrogen atoms form amido functional substitution groups. In one aspect, the amido groups are different. In another aspect, the amido functional groups are the same. The di-amido gellant has the following formula (XXII):

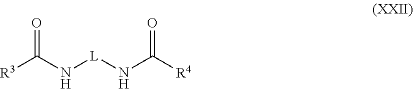

wherein the variables of the di-amido gellant in formula (XXII) are defined as follows:

R³ and R⁴ is an amino functional end-group, or even amido functional end-group, in one aspect

R³ and R⁴ may comprise a pH-tunable group, wherein the pH-tunable amido-gellant may have a pKa of from about 1 to about 30, or even from about 2 to about 10. In one aspect, the pH tunable group may comprise a pyridine. In one aspect, R³ and R⁴ may be different. In another aspect, R³ and R⁴ may be the same.

L is a linking moiety of molecular weight from 14 to 500 g/mol. In one aspect, L may comprise a carbon chain comprising between 2 and 20 carbon atoms. In another aspect, L may comprise a pH-tunable group. In one aspect, the pH-tunable group is a secondary amine. In one aspect, at least one of R³, R⁴ or L may comprise a pH-tunable group.

iv.) Bacterial Cellulose

The term “bacterial cellulose” encompasses any type of cellulose produced via fermentation of a bacteria of the genus Acetobacter such as CELLULON® by CPKelco U.S. and includes materials referred to popularly as microfibrillated cellulose, reticulated bacterial cellulose, and the like.

In one aspect, said fibres may have cross sectional dimensions of 1.6 nm to 3.2 nm by 5.8 nm to 133 nm. Additionally, the bacterial cellulose fibres may have an average microfibre length of at least about 100 nm, or from about 100 to about 1,500 nm. In one aspect, the bacterial cellulose microfibres may have an aspect ratio, meaning the average microfibre length divided by the widest cross sectional microfibre width, of from about 100:1 to about 400:1, or even from about 200:1 to about 300:1.

In one aspect of the invention, the bacterial cellulose is at least partially coated with a polymeric structuring agents (see i. above). In one aspect the at least partially coated bacterial cellulose comprises from about 0.1% to about 5% w/w, or even from about 0.5% to about 3% w/w of bacterial cellulose; and from about 10% to about 90% w/w of a polymeric structuring agent relative to the total weight of the detergent composition. Suitable bacterial cellulose may include the bacterial cellulose described above and suitable polymeric structuring agents include carboxymethylcellulose, cationic hydroxymethylcellulose, and mixtures thereof.

v.) Cellulose Fibers Non-Bacterial Cellulose Derived

Cellulosic fibers may be extracted from vegetables, fruits or wood. Commercially available examples are Avicel® from FMC, Citri-Fi from Fiberstar or Betafib from Cosun.

vi.) Non-Polymeric Crystalline Hydroxyl-Functional Materials

In one aspect of the invention, the composition may comprise non-polymeric crystalline, hydroxyl functional structurants. Said non-polymeric crystalline, hydroxyl functional structurants may comprise a crystallizable glyceride which can be pre-emulsified to aid dispersion into the final liquid detergent composition.

In one aspect, crystallizable glycerides may include hydrogenated castor oil or “HCO” or derivatives thereof, provided that it is capable of crystallizing in the liquid detergent composition.

Depending on the physical form, detergent compositions of the invention may comprise one or more hydrotropes. Usually hydrotropes are used to prevent liquid detergent compositions from separating into layers and/or to ensure liquid detergent composition homogeneity. Non-limiting examples of suitable hydrotropes include ammonium, potassium or sodium salts of toluene, xylene, and cumene sulfonates.

Depending on the final application of the detergent composition of the invention, the detergent composition may comprise one or more fabric softening compounds. Fabric softener usually means a laundry additive that gives textiles a soft feel and smooth surface, reduces static electricity and wrinkling, and makes ironing easier. Fabric softeners may be selected cationic quaternary ammonium compounds as disclosed above.

Often fabric softeners are designed for addition to the rinse or drying cycles. However, fabric softening ingredients may also be incorporated in laundry detergent compositions.

Depending on the final application, detergent compositions of the invention may comprise one or more corrosion inhibitors. Non-limiting examples of suitable corrosion inhibitors include sodium silicate, triazoles such as benzotriazoles, bisbenzotriazoles, aminotriazoles, alkylaminotriazoles, phenol derivatives such as hydroquinone, pyrocatechol, hydroxyhydroquinone, gallic acid, phloroglucinol and pyrogallol.

The current invention relates to a method of preparing a detergent composition comprising mixing in no specified order in one or more steps

component (a): at least one boron-containing compound, and

component (b): pentane-1,2-diol and optionally one or more further diols, and

component (c): at least one serine proteases and optionally one or more further enzymes, and component (d): one or more detergent components.

Components (a) and (b) and (c) and (d) are those as described above including their various preferred embodiments.

A liquid composition comprising components (a), (b) and (c)—as a stock solution—may be introduced into a detergent composition comprising one or more detergent components. Introduction of a liquid composition comprising components (a), (b) and (c) (stock solution) may be conducted by the way of dilution into a detergent composition, e.g. by dilution of about 1:10, of about 1:20, of about 1:30, of about 1:40, of about 1:50, of about 1:60, of about 1:70, of about 1:80, of about 1:90, of about 1:100, of about 1:200, of about 1:300, of about 1:400, of about 1:500, or of about 1:1000.

Furthermore, components (a), (b) and (c) may be directly mixed with one or more detergent component(s) to form a detergent composition.

In one embodiment, microcapsules comprising a liquid composition comprising at least components (a) and (b) and (c) is introduced into liquid detergent compositions comprising one or more detergent component(s). In another embodiment, microcapsules comprising said liquid composition are e.g. spray-dried and introduced into solid detergent compositions.

In one embodiment, the composition comprising at least components (a) and (b) and (c) when converted to an anhydrous form e.g. by lyophilization or spray-drying e.g. in the presence of a carrier material to form aggregates, are introduced into solid or liquid detergent compositions comprising one or more detergent component(s).

“Physical form” of the detergent composition of the invention includes liquid and solid detergent compositions.

Detergent compositions of the invention may be liquid detergent compositions. Detergent compositions which are liquid according to the invention, are liquid at 20° C. and 101.3 kPa. In the context of the present invention, gel-type liquid laundry detergents are a special embodiment of liquid laundry detergents. Gel-type liquid laundry detergents usually contain at least one viscosity modifier, and they contain little or no non-aqueous solvents. Gel-type liquid laundry detergents can be directly applied to stains in soiled laundry.

In one embodiment of the present invention, liquid detergent compositions according to the present invention have a dynamic viscosity in the range of from 500 to 20,000 mPa·s, determined at 25° C. according to Brookfield, for example spindle 3 at 20 rpm with a Brookfield viscosimeter LVT-II.

In one embodiment of the present invention, liquid detergent compositions according to the present invention may have a water content in the range of from 50 to 98% by weight, preferably up to 95%.

In one embodiment of the present invention, liquid detergent compositions according to the present invention may have a total solids content in the range of from 2 to 50% by weight, preferably 10 to 35% by weight.

In one embodiment of the present invention, liquid detergent compositions according to the present invention may comprise solvents other than water (i.e. organic solvent), for example ethanol, n-propanol, iso-propanol, n-butanol, iso-butanol, sec.-butanol, ethylene glycol, propylene glycol, 1,3-propane diol, butane diol, glycerol, diglycol, propyl diglycol, butyl diglycol, hexylene glycol, ethylene glycol methyl ether, ethylene glycol ethyl ether, ethylene glycol propyl ether, and phenoxyethanol, preferred are ethanol, isopropanol or propylene glycol. Liquid detergent compositions according to the present invention may comprise 0.5% to 12% by weight of organic solvent, referring to the total respective liquid detergent composition. In embodiments in which the inventive liquid detergent composition is provided as unit dose, e.g., in form of a pouch, the content of organic solvent may be in the range of 8% to 25% by weight, referring to the total respective liquid detergent composition.

Detergent compositions of the invention may be solid detergent compositions. Solid detergent compositions within this invention means detergent compositions being solid at 20° C. and 101.3 kPa. Solid detergent compositions may be powders or unit doses for laundering, for example tablet.

Solid detergent composition according to the present invention may have residual moisture in the range of 0.1 to 10% by weight, referring to their total solids content. Residual moisture is determined by dry weight determination through vaporization.

The detergent composition of the invention may comprise microcapsules comprising

at least one boron-containing compound [i.e component (a) as described above] and

pentane-1,2-diol and optionally one or more further diols [i.e component (b) as described above] and

at least one serine proteases and optionally one or more further enzymes [i.e component (c) as described above].

Such a detergent composition may be liquid or solid.

The detergent composition of the invention may comprise aggregates and/or granules comprising components (a) and (b) and (c) as described above. Such a detergent composition may solid.

The detergent composition of the invention may take the form of a unit-dose product, which is a packaging of a single dose in a packaging made of water-soluble material (i.e. films). Such a packaging may be called pouch.

Pouches can be of any form, shape and material which is suitable for holding the composition, e.g., without allowing the release of the composition from the pouch prior to water contact. The inner volume of a pouch can be divided into compartments. The compartments of the pouch herein defined are closed structures, made from a water-soluble film which enclose a volume space which comprises different components of a composition. Said volume space is preferably enclosed by a water-soluble film in such a manner that the volume space is separated from the outside environment. The term “outside environment” means for the purpose of this invention “anything which cannot pass through the water-soluble film which encloses the compartment and which is not comprised by the compartment”. The term “separated” means for the purpose of this invention “physically distinct, in that a first ingredient comprised by a compartment is prevented from contacting a second ingredient if said second ingredient is not comprised by the same compartment which comprises said first ingredient”.

A water-soluble film typically has a solubility of at least 50%, preferably at least 75% or even at least 95%, as measured by the following gravimetric method: 10 grams 0.1 gram of material is added in a 400 ml beaker, whereof the weight has been determined, and 245 ml 1 ml of distilled water is added. This is stirred vigorously on magnetic stirrer set at 600 rpm, for 30 minutes. Then, the mixture is filtered through a folded qualitative sintered-glass filter with the pore sizes as defined above (max. 50 micron). The water is dried off from the collected filtrate by any conventional method, and the weight of the remaining polymer is determined (which is the dissolved or dispersed fraction). Then, the % solubility or dispersability can be calculated. Preferred films are polymeric materials, preferably polymers which are formed into a film or sheet. The film can for example be obtained by casting, coating, blow-moulding, extrusion or blow extrusion of the polymer material, as known in the art. Preferred polymers, copolymers or derivatives thereof are selected from polyvinyl alcohols, polyvinyl pyrrolidone and its water-soluble N-vinylpyrrolidone copolymers, polyalkylene oxides, acrylamide, acrylic acid, cellulose, cellulose ethers, cellulose esters, cellulose amides, polyvinyl acetates, polycarboxylic acids and salts, polyaminoacids or peptides, polyamides, polyacrylamide, copolymers of maleic/acrylic acids, polysaccharides including starch and gelatine, natural gums such as xanthum and carragum. More preferably the polymer is selected from polyacrylates and water-soluble acrylate copolymers, methylcellulose, carboxymethylcellulose sodium, dextrin, ethylcellulose, hydroxyethyl cellulose, hydroxypropyl methylcellulose, maltodextrin, polymethacrylates, most preferably polyvinyl alcohols, polyvinyl alcohol copolymers and hydroxypropyl methyl cellulose (HPMC). Mixtures of polymers can also be used. This may in particular be beneficial to control the mechanical and/or dissolution properties of the compartments or pouch, depending on the application thereof and the required needs. For example, it may be preferred that a mixture of polymers is present in the film, whereby one polymer material has a higher water-solubility than another polymer material, and/or one polymer material has a higher mechanical strength than another polymer material.

The pouch can be prepared according to methods known in the art.

The pouches can comprise a solid detergent composition according to the invention and/or a liquid detergent composition according to the invention in different compartments. The compartment for liquid components can be different in composition than compartments containing solids (see e.g., EP 2014756). The composition comprising at least one boron-containing compound [i.e component (a) as described above] and pentane-1,2-diol and optionally one or more further diols [i.e component (b) as described above] and at least one serine proteases and optionally one or more further enzymes [i.e component (c) as described above] may be comprised in either the liquid or the solid detergent composition. In one embodiment, the liquid composition comprising at least components (a) and (b) and (c) additionally comprises one or more pH adjusting compounds and/or one or more preservatives as described above. In one embodiment, the composition comprising at least components (a) and (b) and (c) as such may be enclosed in one compartment of a pouch.

A unit dose product herein also means a solid detergent composition provided as e.g. an extruded pellet, or a tablet having a size of between approximately 1 gram and approximately 250 grams, such as e.g. about 30 g to about 125 g, about 30 g to about 100 g such as e.g. about 30 g to about 75 g. Tablets may also be formed by compression of the components of the detergent composition so that the tablets produced are sufficiently robust to be able to withstand handling and transportation without sustaining damage. In addition to being robust, tablets must also dissolve sufficiently fast so that the detergent components are released into the wash water as soon as possible at the beginning of the wash cycle.

Solid detergent compositions for unit dose solid blocks may comprise a solidification matrix. The solidification matrix generally includes an alkali metal hydroxide alkalinity source, a hydratable salt, such as sodium carbonate (soda ash), a polycarboxylic acid polymer and a water charge for forming solid compositions. Furthermore, other excipient compounds may be used in aiding the tableting preparation. Non-limiting examples of suitable compounds include magnesium stearate, magnesium stearyl fumarate, sodium sulphate (anhydrous), magnesium sulphate (anhydrous), sodium carbonate (anhydrous), magnesium carbonate (anhydrous).

The rate of dissolution at certain cleaning temperatures can be modified by the hardness/density of the tablet. In order to have an anti-caking effect, at least one anti-caking agent such as Mg-silicates, Al-silicates, Na-aluminosilicates is present in the composition.

A tablet may comprise one or more polymeric disintegrants, preferably crosslinked disintegrants. A tablet may also comprise one or more disintegration retardants incorporating the cross-linked polymeric disintegrant. Thereby, different phases may be formed which help to control the dissolution of the various phases at different point in times during the cleaning process.

Suitable cross-linked polymeric disintegrants for use herein include cross-linked starches, cross-linked cellulose ethers, cross-linked polyvinylpyrrolidones, preferably the so-called “polyvinlypyrrolidone-popcorn-polymers” or “PVPP”, cross-linked carboxy-substituted ethylenically-unsaturated monomers, cross-linked polystyrene sulphonates and mixtures thereof. Highly preferred are the cross-linked polyvinylpyrrolidones such as PVPP. Suitable cross-linking agents include bi- and multi-functional linking moieties selected from divinyl and diallyl cross-linkers, polyols, polyvinylalcohols, polyalkylenepolymines, ethyleneimine containing polymers, vinylamine containing polymers and mixtures thereof. Alternatively, the popcorn-polymers such as PVPP can be obtained by the so-called proliferous polymerisation (also “popcorn polymerisation”) with the use of suitable crosslinking monomers.

The particle size and particle size distribution of the cross-linked polymeric disintegrant is important for controlling both the disintegration performance and the stability of tablets during transport and storage. In a preferred embodiment, the polymeric disintegrant has a particle size distribution such that at least about 40%, preferably at least about 50%, more preferably at least about 55% by weight thereof falls in the range of 250 to 850 microns, with less than about 40%, preferably less than about 30% greater than 850 microns, such a distribution being preferred from the view point of providing optimum disintegration and stability profiles.

A tablet may comprise one or more non-cross-linked polymeric disintegrants. Preferred non-crosslinked polymeric disintegrants have a particle size distribution such that at least 90% by weight of the disintegrant has a particle size below about 0.3 mm and at least 30% by weight thereof has a particle size below about 0.2 mm. Suitably, the non-crosslinked polymeric disintegrant is selected from starch, cellulose and derivatives thereof, alginates, sugars, swellable clays and mixtures thereof.

In multi-phase tablets, controlled dissolution characteristics can also be achieved by suitable selection of the level (concentration) of disintegration retardant in the various tablet phases. Thus, according to a further aspect of the invention, there is provided a detergent tablet for use in a washing machine, the detergent tablet comprising a plurality of compressed phases having differing concentrations of disintegration retardant in at least two of the phases and at least one of which phases comprises a cross-linked polymeric disintegrant such as to provide differential dissolution of the two or more phases in a washing machine. Preferably the disintegration retardant has a concentration (relative to the corresponding phase) differing by at least about 5%, more preferably at least about 20% and especially at least about 50% in the at least two phases.

Suitable disintegration retardants herein include but are not limited to organic and other binders, gels, meltable solids, waxes, solubility-triggers (e.g. responsive to pH, ion concentration or temperature), moisture sinks (for example hydratable but anhydrous or partially hydrated salts), viscous or mesophase-forming surfactants, and mixtures thereof. Particularly preferred disintegration retardants herein include amine oxide surfactants, nonionic surfactants, and mixtures thereof. Preferred amine oxide for use herein are tetradecyl dimetyl amine oxide, hexadecyl dimethyl amine oxide and mixtures thereof.

Preferably, the enzyme comprising phase disintegrates early in the cleaning process. In multiphase tablets, preferred detergent components of the first phase to be disintegrated include one or more builders, one or more surfactants, one or more enzymes, optionally one or more bleaching agents, and one or more disintegrants. This enzyme comprising phase may comprise microcapsules of the invention, which have been dried e.g. by spray-drying for the purpose of being incorporated into tablets. The enzyme comprising phase may comprise enzymes in aggregates or granules according to the invention.

Preferred detergent components of the subsequent phases to be disintegrated include one or more builders, one or more enzymes, one or more disintegrants and optionally one or more disintegration retardants.

In a preferred aspect of the present invention the first phase to be disintegrated weighs more than 4 g. More preferably said first phase weighs from 10 g to 30 g, even more preferably from 15 g to 25 g and most preferably form 18 g to 24 g. The subsequent phases to be disintegrated weigh less than 4 g. More preferably the second and/or optional subsequent phases weigh between 1 g and 3.5 g, most preferably from 1.3 g to 2.5 g.

Stain removal and cleaning method using a composition comprising at least one boron-containing compound [i.e component (a) as described above] and pentane-1,2-diol and optionally one or more further diols [i.e component (b) as described above] and at least one serine proteases and optionally one or more further enzymes [i.e component (c) as described above]:

The current invention relates to the use of and method of using compositions comprising component (a) as described above, component (b) as described above, and component (c) as described above, comprising the step of contacting an object to be cleaned with a composition of the invention under conditions suitable for cleaning said object. In one embodiment, the object to be cleaned is contacted with a detergent composition of the invention. The object to be cleaned textiles and/or hard surfaces, such as glass, metallic surfaces including cutlery or dishes.

The invention also relates to the use of and method of using compositions comprising component (a) as described above, component (b) as described above, and component (c) as described above, for removing enzyme-sensitive stains such as proteinaceous stains. Non-limiting examples of proteinaceous stains include stains originating from body fluid such as blood, dairy products such as milk, infant formula, eggs, vegetables, body soils, grass and mud.

In one embodiment, the invention relates to a method of removing enzyme-sensitive stains such as proteinaceous stains from textiles or hard surfaces, such as glass, metallic surfaces including cutlery or dishes.

The current invention relates to a method of cleaning comprising the steps of contacting an object to be cleaned with a composition comprising component (a) as described above, component (b) as described above, and component (c) as described above, under conditions suitable for cleaning said object. In one embodiment, the object to be cleaned is contacted with a detergent composition of the invention. The method of cleaning may be a laundering or hard surface cleaning. The object to be cleaned textiles and/or hard surfaces, such as glass, metallic surfaces including cutlery or dishes.

In one embodiment, the invention relates to a method of treating textiles or hard surfaces, such as glass, metallic surfaces including cutlery or dishes, using compositions comprising component (a) as described above, component (b) as described above, and component (c) as described above for removing proteinaceous stains. Non-limiting examples of proteinaceous stains include stains originating from body fluid such as blood, dairy products such as milk, infant formula, eggs, vegetables, body soils, grass and mud.

### EXAMPLES

Proteolytic activity of proteases has been measured using succinyl-Ala-Ala-Pro-Phe-p-nitroanilide (Suc-AAPF-pNA, short AAPF) as substrate, wherein pNA is cleaved from the substrate molecule by proteolytic cleavage, resulting in release of yellow color of free pNA which was quantified by measuring OD₄₀₅.

If not indicated otherwise, enzymatic activity was measured at 30° C.

Where concentrations of diol and/or borate and/or enzyme are provided by % w/w, the % w/w is in relation to the total weight of the composition tested.

### Example 1: Inhibition of Protease Activity by Diol in the Presence of Boron-Containing Compound

Test Samples:

50 mM phosphate buffer (pH 7.5), 4.4 μM 4-FPBA, 4.8 mM Suc-AAPF-pNA, and 16 nM protease according SEQ ID No: 1 with 2.3% w/w diol or without diol.

The protease activity was measured in presence of the Suc-AAPF-pNA substrate and the 4-FPBA only, which gives the 100% value in Table A.

The protease activity was also measured in the presence of AAPF-pNA substrate, 4-FPBA and 2.3% diol.

TABLE A

| 2.3% (w/w) | | Ethylen-1,2- | Propane- | Butane- | Pentane- | |
|---|---|---|---|---|---|---|
| Diol | — | glycol | 1,2-diol | 1,2-diol | 1,2-diol | Fructose |
| protease | 100% | 54% | 91% | 43% | 29% | 98% |
| activity | | | | | | |

In the presence of BBA and diol, protease had reduced proteolytic activity, meaning protease is inhibited in its proteolytic activity by diol. From Table A it can be concluded that the addition of diol increases the inhibitory effect of 4-FPBA towards proteolytic activity; pentane-1,2-diol results in a strong inhibitory effect when compared to the other diols used.

### Example 2: Inhibition of Protease Activity by Diol Alone

To test the effect of diol alone towards the storage stability of proteases, a protease according to SEQ ID No: 1 was stored at 45° C. with varying concentrations of Propane-1,2-diol and Pentane-1,2-diol as indicated in Table B. 4% active protease according to SEQ ID No: 1 was present before storage for the assessment of its changes in proteolytic activity during storage.

Proteolytic activity was measured in the presence of 4.8 mM Sus-AAPF-pNa. As buffering system 50 mM phosphate buffer was used (pH 7.5). Before storage (time=0), the protease activity measured was set 100%. Protease activity was then measured after storage (at 45° C.) at points in time as indicated in Table B.

TABLE B

| | | proteolytic activity before and after storage | | | | |
|---|---|---|---|---|---|---|
| diol | % w/w | cero | 1 day | 3 days | 7 days | 14 days |
| — | — | 100% | 48% | 30% | 17% | 9% |
| Propane-1,2-diol | 20 | 100% | 82% | 64% | 46% | 30% |
| Pentane-1,2-diol | 20 | 100% | 65% | 38% | 17% | 6% |
| Propane-1,2-diol | 30 | 100% | 92% | 80% | 62% | 47% |
| Pentane-1,2-diol | 30 | 100% | 42% | 13% | 2% | 0% |
| Propane-1,2-diol | 50 | 100% | 94% | 92% | 79% | 66% |
| Pentane-1,2-diol | 50 | 100% | 16% | 1% | 0% | 0% |

From Table B it can be concluded, that pentane-1,2-diol alone does not stabilize the protease, but rather destabilizes the protease; increasing amounts of propane-1,2-diol stabilize the protease increasingly.

### Example 3: Inhibition of Protease Activity by Diol in the Presence and Absence of Boron-Containing Compound

Test samples:

50 mM phosphate buffer (pH 7.5), 137 μM BBA, 4.8 mM Suc-AAPF-pNA, and 16 nM protease according SEQ ID No: 1 with 135 mM diol or without diol.

The protease activity was measured in presence of the Suc-AAPF-pNA substrate and in the absence of BBA and diol, which gives the 100% value in Table B.

The protease activity was measured in presence of the Suc-AAPF-pNA substrate and BBA, which gives the stabilization of protease by BBA—see Table C-I.

TABLE C-I

| BBA | protease activity |
|---|---|
| − | 100% |
| + | 66% |

From Table C-I it can be concluded, that BBA stabilizes protease as in the presence of BBA protease had reduced proteolytic activity (inhibition of proteolytic activity).

The protease activity was then measured in presence of the Suc-AAPF-pNA substrate and diol (without BBA), which gives the stabilization of protease by diol—see Table C-II.

TABLE C-II

| Diol | BBA | Diol (135 mM) | protease activity |
|---|---|---|---|
| Glycerine | − | + | 97% |
| Pentane-1,2-diol | − | + | 70% |
| Propane-1,2-diol | − | + | 94% |
| Ethylene-1,2-glycol | − | + | 97% |
| Butane-1,2-diol | − | + | 84% |

In the presence of diol, protease had reduced proteolytic activity, meaning protease is inhibited in its proteolytic activity. From Table C-II in comparison with Table C-I it can be concluded that diol alone has a less pronounced stabilization effect towards proteolytic activity when compared to BBA. Of the diols tested, pentane-1,2-diol has the best inhibitory effect towards proteolytic activity.

The protease activity was then measured in presence of the Suc-AAPF-pNA substrate and BBA and diol, which gives the stabilization of protease by BBA and diol—see Table C-III.

TABLE C-III

| Diol | BBA | Diol (135 mM) | protease activity |
|---|---|---|---|
| Glycerin | + | + | 31% |
| 1,2-Pentandiol | + | + | 15% |
| 1,2-Propandiol | + | + | 40% |
| Ethylenglycol | + | + | 43% |
| 1,2-Butandiol | + | + | 24% |

In the presence of BBA and diol, protease has reduced proteolytic activity, meaning protease is inhibited in its proteolytic activity. From Table C-III in comparison with table B it can be concluded that diol increases the stabilization of protease in the presence of BBA. Of the diols tested, pentane-1,2-diol has the best increasing effect on stabilization of protease.

Furthermore, from comparing the results for pentane-1,2-diol provided in Tables C-I (BBA: 66%), C-II (pentane-1,2-diol: 70%) and C-III (BBA+pentane-1,2-diol: 15%), a synergistic stabilization of BBA and pentane-1,2-diol is apparent.

### Example 4: Mixtures of Diols

Test Samples:

50 mM phosphate buffer (pH 7.5), 4.4 μM 4-FPBA, 4.8 mM Suc-AAPF-pNA, and 16 nM protease according SEQ ID No: 1 with one or two diols.

The protease activity was measured in presence of the Suc-AAPF-pNA substrate and 4-FPBA and one or two diols, which gives the stabilization of protease by 4-FPBA and one or two diols—see Table D.

TABLE D

| | | | | | |
|---|---|---|---|---|---|
| Propane-1,2-diol [mM] | 120 | 96 | 76 | 45 | 0 |
| Pentane-1,2-diol [mM] | 0 | 11.2 | 22.5 | 34 | 120 |
| Total diol [mM] | 120 | 107 | 98.5 | 79 | 120 |
| Protease activity | 91% | 79.5% | 72% | 70% | 45.5% |

The protease activity in presence of the Suc-AAPF-pNA substrate and 4-FPBA only is set 100%.

The results show, that protease activity in presence of the Suc-AAPF-pNA substrate, 4-FPBA and one or two diols is reduced when compared to stabilization with 4-FPBA only. The means that one or two diols increase the stabilization of the protease. Of the diols tested, pentane-1,2-diol alone stabilizes protease best.

From Table D it can be concluded that increasing concentrations of pentane-1,2-diol and decreasing concentrations of propane-1,2-diol in a diol mixture of propane-1,2-diol and pentane-1,2-diol are advantageous for stabilization of protease.

### Example 5: Storage Stability of Protease in Detergent Composition

Model Formulation for Detergent Composition:

| | | Concentration in detergent |
|---|---|---|
| | | model formulation |
| A | Lutensit A-LBS | 15.5 |
| | Edenor K12-18 (coconut fatty acid) | 3.7 |
| | KOH | 3.5 |
| | Lutensol AO 7 | 8.5 |
| | Ethanol | 2 |
| | Add water to 85% | 51.8 |
| B | enzymes | 16 μM protease, 1.8 μM |
| | | amylase, 1.2 μM lipase |
| | Na-borate and diol | concentration in % w/w as |
| | | indicated in the tables below* |
| | Add water to 15% | |

*relative to the total weight of the model formulation (A + B)

The model formulation (100%) consists of 85% A and 15% B. The formulation had a pH of 8.2.

Enzymes Used:

Protease: SEQ ID No: 2

Amylase: Stainzyme™ from Novozymes

Lipase: Lipex™ from Novozymes

After storage at 37° C., samples were diluted by at least the factor of 100 for measuring the proteolytic activity within the formulation. Due to the dilution, the effect of the inhibitor was reversed.

The value 100% in Table E gives the proteolytic activity measured before storage of the formulation.

TABLE E

| % w/w | % w/w | % w/w | proteolytic activity before | | | |
|---|---|---|---|---|---|---|
| pentane- | propane- | Na- | or after storage | | | |
| 1,2-diol | 1,2-diol | borate | cero | 3 days | 10 days | 21 days |
| 0 | 9 | 1 | 100% | 85% | 51% | 14% |
| 0 | 3 | 1 | 100% | 60% | 13% | 0% |
| 2 | 3 | 1 | 100% | 83% | 47% | 19% |
| 0 | 9 | 2 | 100% | 90% | 61% | 34% |
| 0 | 3 | 2 | 100% | 64% | 26% | 5% |
| 2 | 3 | 2 | 100% | 82% | 54% | 35% |
| 0 | 9 | 3 | 100% | 89% | 65% | 45% |
| 0 | 3 | 3 | 100% | 90% | 40% | 13% |
| 2 | 3 | 3 | 100% | 90% | 60% | 45% |

From Table E it can be concluded, that storage in the presence of 1% w/w borate is less effective than storage with 2% w/w borate which is less effective than storage with 3% w/w borate. Further, from Table E it can be concluded, that storage with 2% pentane-1,2-diol and 3% propane-1,2-diol is equally effective as storage with 9% (w/w) propane-1,2-diol. Storage with 3% propane-1,2-diol is less effective when compared to storage with 2% pentane-1,2-diol and 3% propane-1,2-diol or storage with 9% (w/w) propane-1,2-diol.

Furthermore, amylolytic activity was measured after storage of the formulation comprising the protease. Amylolytic activity was measured by the release of the para-nitrophenol (pNP) chromophore from the ethylidene-blocked 4-nitrophenylmaltoheptaoside substrate (EPS-G7). The value 100% in Table F gives the amylolytic activity measured before storage of the formulation at 37° C.

TABLE F

| % w/w | % w/w | % w/w | amylolytic activity | | | |
|---|---|---|---|---|---|---|
| pentane- | propane- | Na- | before or after | | | |
| 1,2-diol | 1,2-diol | borate | cero | 3 days | 10 days | 21 days |
| 0 | 9 | 1 | 100% | 79% | 46% | 23% |
| 0 | 3 | 1 | 100% | 83% | 42% | 25% |
| 2 | 3 | 1 | 100% | 84% | 57% | 38% |

From Table F it can be concluded that a mixture of 2% (w/w) pentane-1,2-diol and 3% (w/w) propane-1,2-diol has a higher stabilizing effect compared to stabilization with propane-1,2-diol alone.

1. A composition comprising
component (a): at least one phenyl boronic acid or derivatives thereof, and
component (b): pentane-1,2-diol and optionally one or more further water-miscible diols
wherein the composition is liquid at 20° C. and 101.3 kPa.

2. The composition according to claim 1, wherein phenyl-boronic acid derivatives are selected from the group consisting of 4-formyl phenyl boronic acid (4-FPBA), 4-carboxy phenyl boronic acid (4-CPBA), 4-(hydroxymethyl) phenyl boronic acid (4-HMPBA), and p-tolylboronic acid (p-TBA).

3. The composition according to claim 1, wherein component (b) is comprised in amounts the range of 10% to 65% relative to the total composition.

4. The composition according to claim 1, wherein the composition further comprises component (c), which comprises at least one serine protease and optionally one or more further enzymes.

5. The composition according to claim 4, wherein the composition comprises component (c) in amounts ranging from 1 g/L to 100 g/L.

6. The composition according to claim 1, the composition has a pH in the range of 7 to 11.5.

7. A detergent composition comprising
component (a): as defined in any one of the preceding claims, and
component (b): as defined in any one of the preceding claims, and
component (c): as defined in any one of the preceding claims, and
component (d): one or more detergent components,
wherein component (b) is comprised in amounts in the range of 2% to 50% w/w relative to the total weight of the composition, and component (c) is comprised in amounts in the range of 0.01 g/L to 20 g/L.

8. A method of preparing the composition according to claim 1
comprising mixing in no specified order in one or more steps
component (a) as defined in any one of the preceding claims, and
component (b) as defined in any one of the preceding claims, and
optionally component (c) as defined in any one of the preceding claims, and
optionally component (d) as defined in claim 8.

9. The method of claim 8, wherein the composition prepared is a detergent composition and wherein at least components (a), (b) and (c) are introduced as a stock solution.

10. A microcapsule comprising the composition according to claim 1,
wherein components (a) and (b) and (c) are part of the core composition of the microcapsule.

11. (canceled)

12. A method for removing stains comprising contacting an enzyme-sensitive stain with the detergent composition according to claim 7.

13. A method for cleaning comprising contacting soiled material with the detergent composition according to claim 7.


SEQUENCE LISTING (2 sequences)

SEQ ID NO: 1 - protein, 269 aa, Bacillus lentus
AQSVPWGISRVQAPAAHNRGLTGSGVKVAVLDTGISTHPDLNIRGGASFVPGEPSTQDGNGHGTHVAGTIAALNNSIGVLGVAPSAELYAVKVLGADGEGAISSIAQGLEWAGNNGMHVANLSLGSPSPSATLEQAVNSATSRGVLVVAASGNSGASSISYPARYANAMAVGATDQNNNRASFSQYGAGLDIVAPGVNVQSTYPGSTYASLNGTSMATPHVAGAAALVKQKNPSWSNVQIRNHLKNTATSLGSTNLYGSGLVNAEAATR

SEQ ID NO: 2 - protein, 269 aa, Bacillus lentus
AQSVPWGISRVQAPAAHNRGLTGSGVKVAVLDTGISTHPDLNIRGGASFVPGEPSTQDGNGHGTHVAGTIAALNNSIGVLGVAPSAELYAVKVLGASGSGSVSSIAQGLEWAGNNGMHVANLSLGSPSPSATLEQAVNSATSRGVLVVAASGNSGAGSISYPARYANAMAVGATDQNNNRASFSQYGAGLDIVAPGVNVQSTYPGSTYASLNGTSMATPHVAGAAALVKQKNPSWSNVQIRNHLKNTATSLGSTNLYGSGLVNAEAATR